SEQUENCE LISTING (18 sequences)

SEQ ID NO: 1 - DNA, 318 bp, Artificial Sequence
  Description of Artificial Sequence Synthetic polynucleotide
  CDS (1)..(318)
caaattgttctcacccagtctccagcaatcatgtctgcatctccaggggagaaggtcaccatgacctgcagtgccagctcaagtgtaagttacatgcactggtaccagcagaagtcaggcacctcccccaaaagatggatttatgacacatccaaactggcttctggagtccctgctcgcttcagtggcagtgggtctgggacctcttactctctcacaatcagcagcatggaggctgaagatgctgccacttattactgccagcagtggagtagtaacccgctcacgttcggtgctgggaccaagctggagctgaaa

SEQ ID NO: 2 - DNA, 348 bp, Artificial Sequence
  Description of Artificial Sequence Synthetic polynucleotide
  CDS (1)..(348)
cagatccagttggtacagtctggacctgagctgaagaagcctggagagacagtcaagatctcctgcaaggcttctgggtataccttcacaacctatggaatgagctgggtgaaacaggctccaggaaagggtttaaagtggatgggctggataaacacctactctggagtgccaacatatgctgatgacttcaagggacggtttgccttctctttggaaacctctgccagcactgcctatttgcagatcaacaacctcaaaaatgaggacacggctacatatttctgtgcaagacattactacggaggggcttactggggccaagggactctggtcactgtctctgca

SEQ ID NO: 3 - protein, 116 aa, Artificial Sequence
  Description of Artificial Sequence Synthetic polypeptide
QIQLVQSGPELKKPGETVKISCKASGYTFTTYGMSWVKQAPGKGLKWMGWINTYSGVPTYADDFKGRFAFSLETSASTAYLQINNLKNEDTATYFCARHYYGGAYWGQGTLVTVSA

SEQ ID NO: 4 - protein, 106 aa, Artificial Sequence
  Description of Artificial Sequence Synthetic polypeptide
QIVLTQSPAIMSASPGEKVTMTCSASSSVSYMHWYQQKSGTSPKRWIYDTSKLASGVPARFSGSGSGTSYSLTISSMEAEDAATYYCQQWSSNPLTFGAGTKLELK

SEQ ID NO: 5 - protein, 7 aa, Artificial Sequence
  Description of Artificial Sequence Synthetic peptide
PTTTPLK

SEQ ID NO: 6 - protein, 9 aa, Artificial Sequence
  Description of Artificial Sequence Synthetic peptide
PPTTTTKKP

SEQ ID NO: 7 - protein, 16 aa, Artificial Sequence
  Description of Artificial Sequence Synthetic peptide
GTTPSPVPTTSTTSAP

SEQ ID NO: 8 - protein, 13 aa, Artificial Sequence
  Description of Artificial Sequence Synthetic peptide
PTTDSTTPAPTTK

SEQ ID NO: 9 - protein, 10 aa, Artificial Sequence
  Description of Artificial Sequence Synthetic peptide
TSAPDTRDAP

SEQ ID NO: 10 - protein, 8 aa, Artificial Sequence
  Description of Artificial Sequence Synthetic peptide
APGSTAPP

SEQ ID NO: 11 - protein, 18 aa, Artificial Sequence
  Description of Artificial Sequence Synthetic peptide
GAKCVAAWTLKAAATTTG

SEQ ID NO: 12 - protein, 4 aa, Artificial Sequence
  Description of Artificial Sequence Synthetic peptide
KTTT

SEQ ID NO: 13 - protein, 5 aa, Artificial Sequence
  Description of Artificial Sequence Synthetic peptide
KTTTG

SEQ ID NO: 14 - protein, 12 aa, Artificial Sequence
  Description of Artificial Sequence Synthetic peptide
KPVPSTPPTPSC

SEQ ID NO: 15 - protein, 12 aa, Artificial Sequence
  Description of Artificial Sequence Synthetic peptide
KPSTPPTPSPSC

SEQ ID NO: 16 - protein, 12 aa, Artificial Sequence
  Description of Artificial Sequence Synthetic peptide
KTPPTPSPSTPC

SEQ ID NO: 17 - protein, 12 aa, Artificial Sequence
  Description of Artificial Sequence Synthetic peptide
KPTPSPSTPPTC

SEQ ID NO: 18 - protein, 12 aa, Artificial Sequence
  Description of Artificial Sequence Synthetic peptide
KPSPSTPPTPSC

(19) United States
(12) Patent Application Publication
(10) Pub. No.: US 2019/0345255 A1
(43) Pub. Date: Nov. 14, 2019

(54) MONOCLONAL IGM ANTIBODIES FROM ENTIRELY CARBOHYDRATE CONSTRUCTS

(71) Applicant: The University of Toledo, Toledo, OH (US)

(72) Inventors: Peter Andreana, Toledo, OH (US); Kevin Trabbic, Toledo, OH (US); Mengchao Shi, Toledo, OH (US); Jean Paul Bourgault, Toledo, OH (US)

(73) Assignee: The University of Toledo, Toledo, OH (US)

(21) Appl. No.: 16/331,301

(22) PCT Filed: Sep. 19, 2017
(86) PCT No.: PCT/US2017/052169

§ 371 Date: Mar. 7, 2019

Related U.S. Application Data
(60) Provisional application No. 62/396,603, filed on Sep. 19, 2016.

(51) Int. Cl. C07K 16/30; A61P 35/00; A61K 39/395; A61K 39/00; C12N 5/12; A61K 47/68; A61K 47/61; A61K 39/39

(52) U.S. Cl. CPC C07K 16/30; A61P 35/00; A61K 39/39558; A61K 39/001173; A61K 39/39; C12N 5/12; A61K 47/6855; A61K 47/61; A61K 39/001172

(57) ABSTRACT

Entirely carbohydrate immunogens, monoclonal antibodies generated from immune responses to entirely carbohydrate immunogens, vaccine compositions, pharmaceutical compositions, and methods of making and using the same, are described.

### RELATED APPLICATIONS

This application claims priority to U.S. Provisional Application No. 62/396,603 filed under 35 U.S.C. § 111(b) on Sep. 19, 2016, the disclosure of which is incorporated herein by reference in its entirety.

### STATEMENT REGARDING FEDERALLY SPONSORED RESEARCH

This invention was made with government support under grant number CA156661 awarded by the National Institutes of Health. The government has certain rights in this invention.

### SEQUENCE LISTING

The instant application contains a Sequence Listing which has been submitted electronically in ASCII format and is hereby incorporated by reference in its entirety. Said ASCII copy, created on Sep. 18, 2017, is named 53-58415-D2015-13_SL.txt and is 9,684 bytes in size.

### BACKGROUND OF THE INVENTION

Aberrant glycosylation, affiliated with certain proteins and glycosyltransferases, is observed in the carcinogenesis of cells, which leads to truncated patterns of oligosaccharides on cancer cell surfaces. These “abnormal” oligosaccharides can serve as biomarkers to distinguish tumor cells from normal healthy cells, and are known as tumor-associated carbohydrate antigens (TACAs). A significant portion of the top rated cancer antigens have been identified as TACAs. Abundantly found on the surface, TACAs represent suitable targets for immunotherapies because they are expressed on virtually all forms of cancer.

The unique biological features of TACAs provides an opportunity for exploiting the immune system in the development of anti-TACA vaccines for cancer immunotherapy. Based on the general theory of vaccination, if exogenous TACA-conjugates can be processed and presented to effector cells of adaptive immunity, then an immune response can be stimulated to generate corresponding antibodies and immune memory. One of the major hurdles in materializing this theory is the immunological nature of carbohydrate epitopes. It is known that TACAs cannot elicit strong T cell dependent immune responses, and have failed to induce class switching in order to produce high affinity IgG antibodies and memory B cells. In order to overcome this deficiency, the introduction of immunological “carriers” is necessary. Antigen “carriers” play an important role in cancer vaccine development. Known “carriers” are immunogenic proteins, such as keyhole limpet hemocyanin (KLH), diphtheria toxin (CRM197), and tetanus toxoid (TT). While there have been positive results with bacterial-based glycoprotein conjugates, two major drawbacks hinder further success for their use in cancer therapy: 1) the immunogenicity of protein carriers may overwhelm that of TACAs, leading to an “epitope suppression” effect, and 2) non-site specific coupling may cause heterogeneities and ambiguities of chemical composition.

The FDA has approved Unituxin, the first monoclonal antibody (mAb) targeting GD2 (GalNAcβ1-4(Neu5AcA2-8Neu5AcA2-3) Galβ1-4Glc)), for the treatment of high risk neuroblastoma in pediatric patients. Unituxin was developed from the immunization with the neuroblastoma cell line, LAN-1. However, before the Unituxin approval, only protein (non-carbohydrate)-based cancer antigens led to the FDA certification of approximately 30 mAbs, including Trastuzumab, Rituximab, and Bevacizumab. Unlike proteins, carbohydrate-based immune responses are T cell independent responses. These limitations can be altered by conjugation to proteins, however, this can still have some disadvantages such as protein epitope suppression and immune responses towards non-natural hydrocarbon linkers. Since there is often ambiguity in the effectiveness of TACA-protein conjugates, new immunogen strategies that target glycosides need to be discovered and examined in order to produce more effective immunotherapies.

Furthermore, many TACA-specific mAbs have cross reactivity to other carbohydrates, and some do not even bind the target all together. The epitome of the lack of carbohydrate specificity is seen with B1.1, which is a commercially available monoclonal IgM specific for Tn but actually fails to bind Tn alone. Rather, it interacts with a cluster of Tn (AcTn-Tn-Tn-Gly-Hex-BSA) antigens. The Tn cluster provides enough surface area for B1.1 to bind due to the strong avidity of monoclonal IgM antibodies. Thus, discovering new strategies for the development of mAbs against TACAs is a challenging but critical aspect in ensuring carbohydrate specificity and selectivity.

### SUMMARY OF THE INVENTION

Provided is a monoclonal antibody comprising a light chain amino acid sequence consisting of:

[SEQ ID NO: 1]
CAAATTGTTCTCACCCAGTCTCCAGCAATCATGTCTGCATCTCCAGGGGA
GAAGGTCACCATGACCTGCAGTGCCAGCTCAAGTGTAAGTTACATGCACT
GGTACCAGCAGAAGTCAGGCACCTCCCCCAAAAGATGGATTTATGACACA
TCCAAACTGGCTTCTGGAGTCCCTGCTCGCTTCAGTGGCAGTGGGTCTGG
GACCTCTTACTCTCTCACAATCAGCAGCATGGAGGCTGAAGATGCTGCCA
CTTATTACTGCCAGCAGTGGAGTAGTAACCCGCTCACGTTCGGTGCTGGG
ACCAAGCTGGAGCTGAAA,

and a heavy chain amino acid sequence of:

[SEQ ID NO: 2]
CAGATCCAGTTGGTACAGTCTGGACCTGAGCTGAAGAAGCCTGGAGAGAC
AGTCAAGATCTCCTGCAAGGCTTCTGGGTATACCTTCACAACCTATGGAA
TGAGCTGGGTGAAACAGGCTCCAGGAAAGGGTTTAAAGTGGATGGGCTGG
ATAAACACCTACTCTGGAGTGCCAACATATGCTGATGACTTCAAGGGACG
GTTTGCCTTCTCTTTGGAAACCTCTGCCAGCACTGCCTATTTGCAGATCA
ACAACCTCAAAAATGAGGACACGGCTACATATTTCTGTGCAAGACATTAC
TACGGAGGGGCTTACTGGGGCCAAGGGACTCTGGTCACTGTCTCTGCA.

Further provided is a composition comprising a murine monoclonal antibody which (i) binds to the glycoside portion of a Tn antigen, and (ii) has the IgM isotype. In certain embodiments, the composition is substantially free of additional peptides or proteins.

Further provided is a composition comprising an antibody raised against an entirely carbohydrate immunogen.

Further provided is a test device, kit, or strip comprising a monoclonal antibody described herein. In certain embodiments, the monoclonal antibody is labeled with one of an enzyme, a fluorescent material, a chemiluminescent material, biotin, avidin, or a radioactive isoptope.

Further provided is a pharmaceutical composition comprising a monoclonal antibody described herein, and a pharmaceutically acceptable carrier, diluent, or adjuvant. Further provided is a method of treating, preventing, or ameliorating a cancer, the method comprising administering an effective amount of the pharmaceutical composition to a subject in need thereof, and treating, preventing, or ameliorating a cancer in the subject. In particular embodiments, the cancer is breast cancer.

Further provided is a STn-PS A1 construct having Formula I:

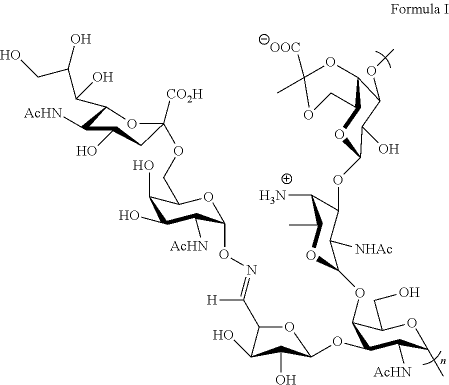

Also provided are salts, stereoisomers, racemates, hydrates, solvates, and polymorphs of Formula I.

Further provided is a composition comprising a carbohydrate immunogen having Formula II:

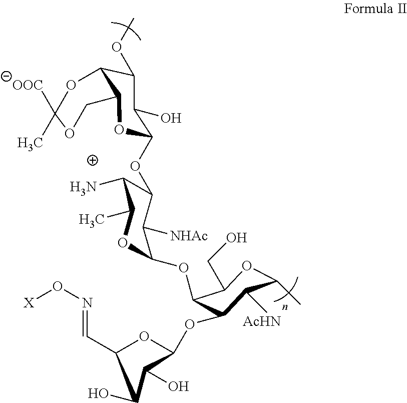

where X is selected from the group consisting of TF, Tn-TF, Gb3, and Globo H. Also provided are salts, stereoisomers, racemates, hydrates, solvates, and polymorphs of Formula II.

Further provided is a vaccine composition comprising an entirely carbohydrate immunogen comprising a zwitterionic polysaccharide conjugated to an STn antigen, a TF antigen, a Globo H antigen, or a conjugate of a TF antigen and a Tn antigen, and a pharmaceutically acceptable carrier, diluent, or adjuvant. Further provided is a method of treating, preventing, or ameliorating a cancer, the method comprising administering an effective amount of the vaccine composition to a subject in need thereof, and treating, preventing, or ameliorating a cancer in the subject. In particular embodiments, the cancer is breast cancer.

Further provided is a method of treating, preventing, or ameliorating a cancer, the method comprising administering monoclonal antibodies to a subject in need thereof, and treating, preventing, or ameliorating a cancer in the subject, wherein the monoclonal antibodies are generated from an immune response to an entiretly carbohydrate immunogen, and the monoclonal antibodies are specific and selective for glycosides of a tumor-associated carbohydrate antigen (TACA). In other words, all or substantially all of the donor/acceptor Fab/antigen binding events are selective and specific towards carbohydrate moieties on the surface of tumor cells. In certain embodiments, the monoclonal antibodies are IgM antibodies. In certain embodiments, the cancer is breast cancer.

Further provided is a method of generating monoclonal antibodies, the method comprising administering an immunogen comprising an entirely carbohydrate construct to an animal to provoke an immune response in the animal and generate antibodies against the entirely carbohydrate construct, wherein the entirely carbohydrate construct comprises a zwitterionic polysaccharide conjugated to a tumor-associated carbohydrate antigen (TACA), harvesting B cells from the animal, fusing the harvested B cells with B cell cancer cells to produce hybridoma cells, culturing the hybridoma cells, and harvesting monoclonal antibodies from the cultured hybridoma cells, where the monoclonal antibodies are selective for glycosides of the TACA. In certain embodiments, the monoclonal antibodies are selective for a Tn antigen. In certain embodiments, the animal is a mouse. In certain embodiments, the entirely carbohydrate construct is a Tn-PS A1 construct.

Further provided is a method of determining health insurance reimbursement or payments, the method comprising denying coverage or reimbursement for a treatment, where the treatment comprises administering a monoclonal antibody described herein, or a vaccine composition described herein, to a patient.

### BRIEF DESCRIPTION OF THE DRAWINGS

The patent or application file may contain one or more drawings executed in color and/or one or more photographs. Copies of this patent or patent application publication with color drawing(s) and/or photograph(s) will be provided by the U.S. Patent and Trademark Office upon request and payment of the necessary fees.

FIG. 1: Non-limiting illustration showing the production of immunotherapeutic mAbs from an entirely carbohydrate immunogen.

FIG. 2: Depiction of non-limiting example zwitterionic polysaccharides.

FIG. 3: Synthesis of Tn-PS A1 by the oxidation and conjugation to PS A1.

FIG. 4: Scheme 1, showing a retrosynthetic analysis of aminooxy sialyl Tn antigen.

FIG. 5: Table 1, displaying the results of sialylation using different galactopyranose acceptors and compound 3 as the donor. Typical conditions: 1.2 equivalents of donor 3, 1.3 equivalents of TMSOTf, dry DCM, and −45° C. for 30 min. [b] Isolated yield. [c] Determined by ¹H NMR spectroscopic analysis of the unpurified reaction mixture. [d] Reaction mixture stirred at −45° C. for 30 min, then gradually warmed to 0° C., and finally stirred for another 45 min to obtain product.

FIG. 6: Scheme 2, showing the synthesis of α-aminooxy STn antigen (1).

FIG. 7: Scheme 3, showing the preparation of STn-PS A1 conjugate (16).

FIG. 8: Comparison of ¹H NMR of PS A1 (15) and STn-PS A1 (16).

FIGS. 9A-9B: ELISA analysis of antisera induced by STn-PS A1+SAS, STn-PS A1+TMG, and STn-PS A1 against BSM: group average IgG (FIG. 9A), and group average IgM (FIG. 9B). Control sera obtained from non-immunized mice. The error bars represented standard deviation (SD) of two triplicate tests.

FIG. 10: ELISA analysis of anti-PS A1 antibody induced by STn-PS A1+SAS, anti-STn response determined by using BSM coating, anti-PS A1 response determined by using PS A1-PPL coating. The error bars represent the standard deviation (SD) of two triplicate tests.

FIG. 11: Determination of isotypes and subclasses of antibodies induced by STn-PS A1+SAS, STn-PS A1+TMG, and STn-PS A1. The error bars represent the standard deviation (SD) of two triplicate tests.

FIGS. 12A-12D: FACS analysis of IgG tumor cell binding: MCF-7 (FIG. 12A) and OVCAR-5 (FIG. 12C). IgM tumor cell binding: MCF-7 (FIG. 12B) and OVACR-5 (FIG. 12D).

FIG. 13: Antibodies raised against STn-PS A1+SAS mediate complement-dependent cytotoxicity (CDC) to kill STn containing tumor cells. The cytotoxicity was determined using the commercially available LDH assay. Data shown are mean values of two parallel triplicate tests, where *P<0.01 and **P<0.001 were obtained using a Student's t-test, where # P>0.5 was obtained. The error bars represent the standard deviation (SD) of two triplicate tests.

FIGS. 14A-14B: Synthetic modification of PS A1 (21) (FIG. 14B), and ¹H NMR overlay of PS A1 conjugates 21 and 24a-24c at 60° C. in D₂O (FIG. 14B).

FIGS. 15A-15D: ELISA specificity of TACA-conjugates (24a-24c). FIG. 15A shows IgG specificity towards Tn-BSA. FIG. 15B shows IgM specificity towards Tn-BSA. FIG. 15C shows IgG specificity towards TF-BSA. FIG. 15D shows IgM specificity towards TF-BSA. Both PS A1 and PBS control mice sera had no cross-reactivity to either Tn-BSA or TF-BSA.

FIG. 16: Scheme Y1, showing the syntheses of biotinylated TACA-PS A1 (25a-25c) from TACA-conjugates (24a-24c) as MGL2 assay probes.

FIGS. 17A-17B: Graphs showing MGL2 binding assay and inhibition using probes 25a-25d (Scheme Y1) (FIG. Y4A), and percent inhibition by Tn-BSA (10 μg/mL) with 25a-25c (10 μg/mL) (FIG. 17B). * denotes % inhibition by Tn-BSA.

FIGS. 18A-18B: Flow cytometry with anti-serum from 1 and 24a-24c with secondary Alexa Fluor® 488 anti-IgG using human tumor cell lines. FIG. 18A shows MCF-7 human breast tumor cell line. FIG. 18B shows OVCAR-5 human ovarian tumor cell line.

FIGS. 19A-19B: Antibody mediated CDC with anti-serum from 1 and 24a-24c plus rabbit complement. FIG. 19A shows MCF-7 human breast tumor cell line. FIG. 19B shows OVCAR-5 human ovarian tumor cell line. *P<0.05, **P<0.005, ***P<0.0005.

FIG. 20: Structure of Tn-PS A1.

FIGS. 21A-21B: Graphs showing titration of Kt-IgM-8 on ELISA.

FIGS. 22A-22B: Carbohydrate specificity for Kt-IgM-8 using varying sugar moieties and oligomers (FIG. 22A), and results of Kt-IgM-8 and Tn-218 binding to Tn-BSA (FIG. 22B).

FIGS. 23A-23B: Flow Cytometry of Kt-8-IgM binding to MCF-7 (FIG. 23A) and HCT-116 (FIG. 23B),

FIG. 24: CDC activity of KT-IgM-8 on MCF-7 cells. Data are illustrated as mean±s.e.m. **P<0.005, ***P<0.0005; two tailed Student's t-test.

FIGS. 25A-25E: Kt-IgM-8 displays tumor volume (mm³) reduction of MCF-7 tumors in SCID mice for 39 days. FIG. 25A shows KT-IgM-8 treatment of MCF-7 tumor growth in comparison to PBS control mice. FIG. 25B shows anti-Tn-PS A1 whole sera in comparison to PBS mice over. FIG. 25C shows anti-Tn-PS A1 pIgG in comparison to PBS mice. FIG. 25D shows tumor volume at day 39. FIG. 25E shows tumor volume at day 44. Data are illustrated as mean±s.e.m. **P<0.005, ***P<0.0005; two tailed Student's t-test.

FIG. 26: ¹H NMR of Tn-PS A1.

FIG. 27: ¹H NMR of TF-PS A1.

FIG. 28: ¹H NMR of Tn-TF-PS A1 (24c).

FIG. 29: Expansion ¹H NMR of Tn-TF-PS A1 (24c).

FIG. 30: Expansion ¹H NMR of Tn-TF-PS A1 (24c).

FIG. 31: Structures of ZPS PS A1 (51) and PS B (52) from B. fragilis.

FIG. 32: Scheme showing the production of a TF-PS B (54) immunogen.

FIG. 33: Sialic acid determination using periodate-rescorinol assay.

FIG. 34: Table 2, evaluating PS B (52) and TF-PS B (54) constructs through immunizations.

FIG. 36: Reaction of TF-ONH₂ with Maleic Anhydride (MA) coated ELISA plates to observe IgG immune response from TF-BSA and TF-PS B as a comparison. The plates were blocked with 2% casein to avoid reactivity with anti-BSA sera.

FIG. 37: Scheme 5, showing production of TF-BSA conjugate.

FIGS. 38A-38D: IgG tumor cell binding for MCF-7 (FIG. 38A, blue line) and HCT-116 (FIG. 38B, blue line), and IgM tumor cell binding for MCF-7 (FIG. 38C) and HCT-116 (FIG. 38D). N.B. Serum IgG antibodies were detected using commercially available 2o Alexa Fluor488® anti IgG antibody. Serum IgM antibodies were detected using commercially available 2o Alexa Fluor647® anti IgM antibody.

FIGS. 39A-398D: Cytotoxicity of MCF-7 using TF-PS B. Schematic representation of ADCC. (FIG. 39A.) MCF-7 ADCC with TF-PS B. (FIG. 39B). Schematic representation of CDC. (FIG. 39C.) MCF-7 CDC with TF-PS B. (FIG. 39D.)

FIG. 40: Synthesis of GH-PS A1 conjugates 91a-91c: Globo H-PS A1 (91a), bivalent Tn-GH-PS A1 (91b), and GB3-PS A1 (91c).

FIGS. 41A-41D: The IgG and IgM immune response from Globo H conjugates: Anti-IgIG (GH-BSA) (FIG. 41A), Anti-IgM (GH-BSA) (FIG. 41B), IgG binding GH-BSA (FIG. 41C), and IgM binding GH-BSA (FIG. 41D).

FIGS. 42A-42D: Cross reactivity of IgG (FIGS. 42A, 42C) and IgM (FIGS. 42B, 42D) antibodies from Globo H-PS A1 conjugates to GB3-BSA.

FIG. 43: The immune response generated from GB3-PS A1 and recognition of GB3-BSA.

FIGS. 44A-44D: Cross reactivity of anti-serum (1:100 dilution) of GH-PS A1 constructs to blood group A (FIGS. 44A-44B) and blood group B (FIGS. 44C-44D).

FIGS. 45A-45C: Flow cytometry with anti-serum from PS A1, Globo H-PS A1, and Tn-PS A1 with secondary Alexa Fluor® 488 anti-IgG using the human tumor cell lines MCF-7 breast tumor cell line (FIG. 45A) and OVCAR-5 ovarian tumor cell line (FIG. 45B). FIG. 45C shows a summary of this data.

FIGS. 46A-46B: Antibody mediated CDC with anti-serum from PS A1, Globo H-PS A1, and Tn-PS A1 plus rabbit complement for MCF-7 human breast tumor cell line (FIG. 46A) and OVCAR-5 human ovarian tumor cell line (FIG. 46B).

FIGS. 47A-48B: Heavy (FIG. 47A (SEQ ID NOS 2 and 3, respectively, in order of appearance)) and light (FIG. 47B (SEQ ID NOS 1 and 4, respectively, in order of appearance)) chain sequencing of Kt-IgM-8.

FIGS. 48A-48C: Graph (FIG. 48A) showing the glycan binding specificity of Kt-IgM-8 to various glycopeptides at antibody amounts of 2 μg, and Table 3 (FIGS. 48B-48C) displaying a summary of the glycopeptide array data depicted in FIG. 48A by chart ID number and structure.

FIGS. 49A-49C: Graph (FIG. 49A) showing the glycan binding specificity of Kt-IgM-8 to various glycopeptides at antibody amounts of 20 μg, and Table 4 (FIGS. 49B-49C) displaying a summary of the glycopeptide array data depicted in FIG. 49A by chart ID number and structure.

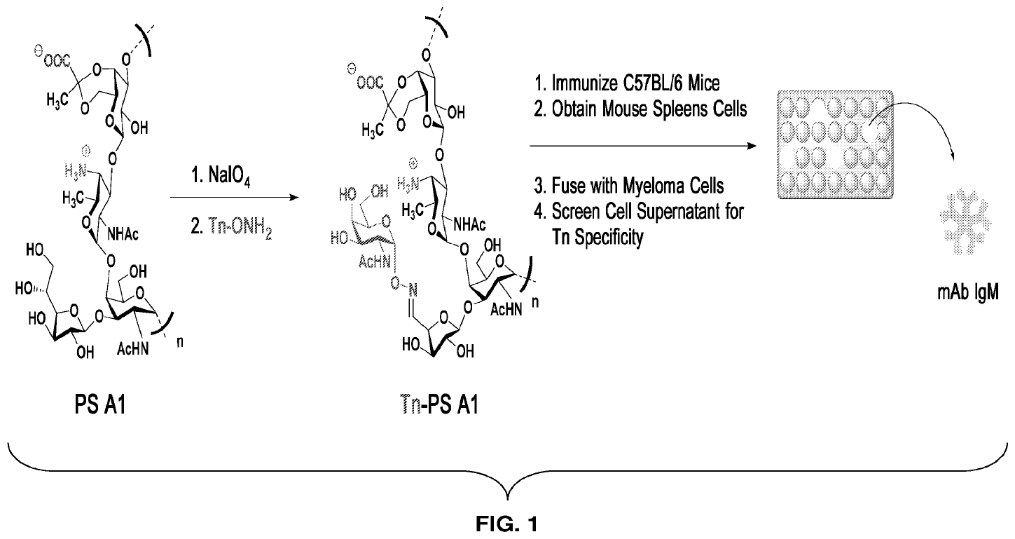

### DETAILED DESCRIPTION OF THE INVENTION

Throughout this disclosure, various publications, patents, and published patent specifications are referenced by an identifying citation. The disclosures of these publications, patents, and published patent specifications are hereby incorporated by reference into the present disclosure in their entirety to more fully describe the state of the art to which this invention pertains.

Most of the FDA approved antibodies approved are IgG. However, IgM antibodies are useful because of their industrial purification and for their ability to initiate complement directed cytotoxicity (CDC) as the main mechanism of cytotoxicity. Additionally, targeting specific glycosides on carcinomas, including the Tn antigen, has therapeutic benefit for binding monoclonal IgM antibodies due to greater avidity towards glycosides. There are numerous examples of mAbs (IgG and IgM) that can recognize TACAs and specifically the Tn antigen, but they lack accurate specificity to glycosides. Thus, provided herein are monoclonal IgM antibodies from entirely carbohydrate constructs that bind to the Tn (alpha-D-GalNAc) cancer antigen. The Tn antigen is a tumor associated carbohydrate antigen (Tn) and is present on a majority of all cancers (80-90%).

Monoclonal antibodies (mAb) are an infinite source of a specific antibody that come from immunized mice and immortalized spleen cells. Monoclonal antibodies are useful in cancer therapeutics, namely immunotherapy by specifically binding to cancer cells. The IgM antibodies described herein have the ability to bind to known cancer cells in flow cytometry and demonstrate complement mediated killing of cancer cells, in vivo and in vitro. These mAbs can be produced in large scale from entirely carbohydrate-based antigens.

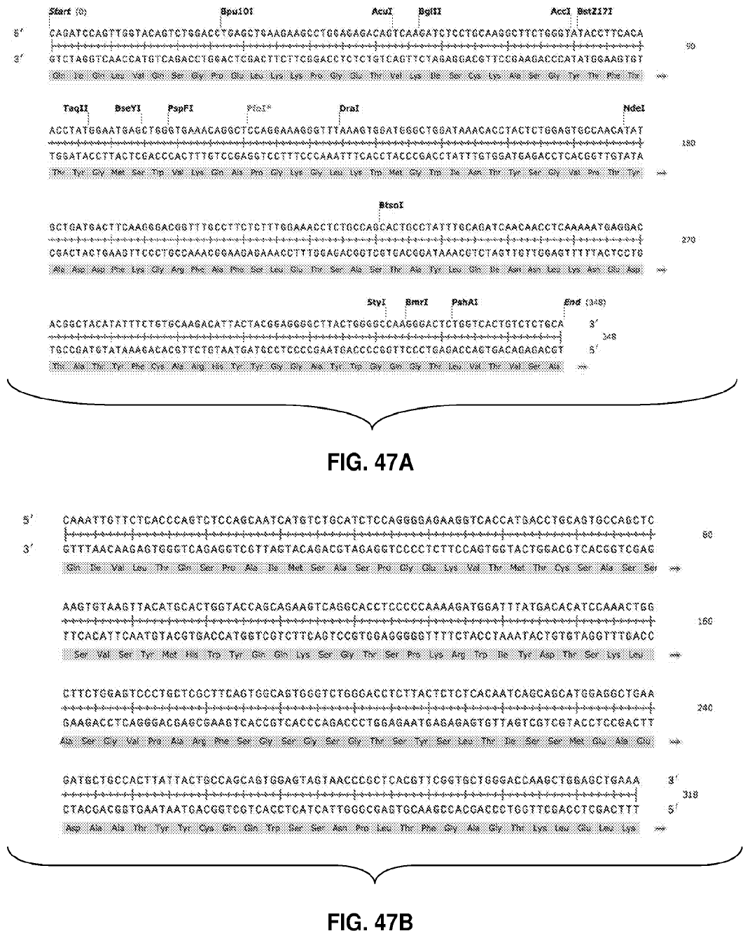

Provided is a monoclonal IgM antibody, named Kt-IgM-8, specific and selective for the TACA Tn antigen. The heavy and light chain sequences of Kt-IgM-8 are shown in FIG. 47A and FIG. 47B, respectively. Kt-IgM-8 has a light chain amino acid sequence of

[SEQ ID NO: 1]
CAAATTGTTCTCACCCAGTCTCCAGCAATCATGTCTGCATCTCCAGGGGA
GAAGGTCACCATGACCTGCAGTGCCAGCTCAAGTGTAAGTTACATGCACT
GGTACCAGCAGAAGTCAGGCACCTCCCCCAAAAGATGGATTTATGACACA
TCCAAACTGGCTTCTGGAGTCCCTGCTCGCTTCAGTGGCAGTGGGTCTGG
GACCTCTTACTCTCTCACAATCAGCAGCATGGAGGCTGAAGATGCTGCCA
CTTATTACTGCCAGCAGTGGAGTAGTAACCCGCTCACGTTCGGTGCTGGG
ACCAAGCTGGAGCTGAAA,

and a heavy chain amino acid sequence of

[SEQ ID NO: 2]
CAGATCCAGTTGGTACAGTCTGGACCTGAGCTGAAGAAGCCTGGAGAGAC
AGTCAAGATCTCCTGCAAGGCTTCTGGGTATACCTTCACAACCTATGGAA
TGAGCTGGGTGAAACAGGCTCCAGGAAAGGGTTTAAAGTGGATGGGCTGG
ATAAACACCTACTCTGGAGTGCCAACATATGCTGATGACTTCAAGGGACG
GTTTGCCTTCTCTTTGGAAACCTCTGCCAGCACTGCCTATTTGCAGATCA
ACAACCTCAAAAATGAGGACACGGCTACATATTTCTGTGCAAGACATTAC
TACGGAGGGGCTTACTGGGGCCAAGGGACTCTGGTCACTGTCTCTGCA.

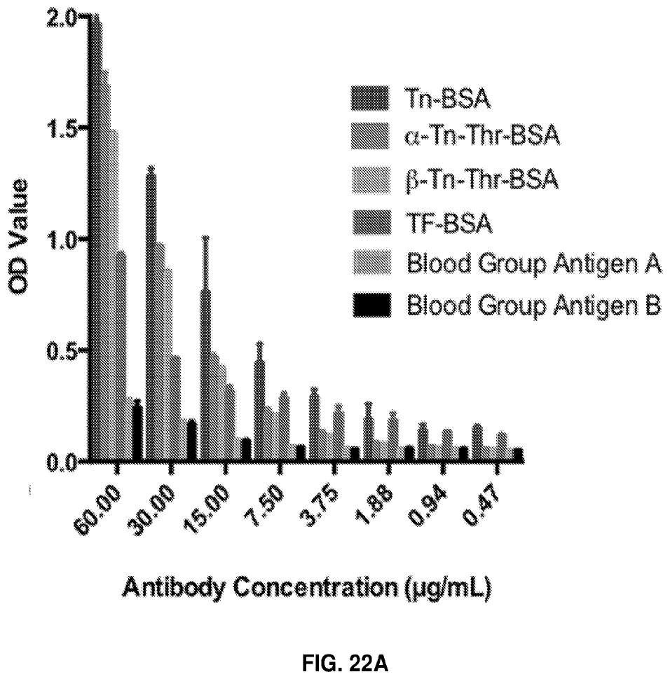

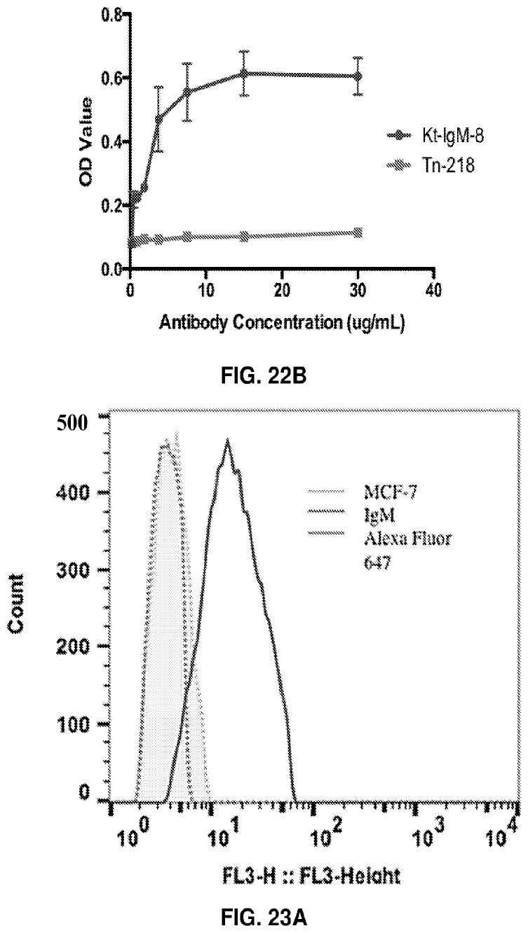

This mAb has demonstrated exceptional binding to the glycoside portion of the Tn antigens in ELISA. To illustrate the effectiveness of Kt-IgM-8, a commercial mAb (clone Tn-218) was compared and determined to be less effective at recognizing the Tn antigen than Kt-IgM-8 (FIG. 22B). The advantage of this antibody compared to Tn-218 is that Kt-IgM-8 can specifically recognize Tn without assistance from peptides or proteins, or a combination of both peptides and proteins.

Amino acid sequence variants of the mAb Kt-IgM-8 are also encompassed within the present disclosure. Modifications to the mAb can be introduced by peptide synthesis. Such modifications include, for example, deletions from, insertions into, and/or substitutions within the amino acid sequence of Kt-IgM-8. Any combination of deletion, insertion, and substitution can be made to arrive at the final amino acid sequence of the antibody, provided that the final antibody possesses the desired biological activity, namely, the binding characteristics of Kt-IgM-8 (i.e., selectivity and specificity for the glycoside portion of the Tn antigen). Accordingly, provided herein are variants of the monoclonal antibodies described. In some embodiments, the variants include an antibody variant having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence of Kt-IgM-8. Reference to a “% sequence identity” with respect to a reference polypeptide is defined as the percentage of amino acid residues in a candidate sequence that are identical with the amino acid residues in the reference polypeptide, after aligning the sequences and introducing gaps, if necessary, to achieve the maximum percent sequence identity, and not considering any conservative substitutions as part of the sequence identity.

It is understood that IgM antibodies can be converted into IgG antibodies through methods known in the art. For example, a reductant can be used to cleave the disulfide bridges of the IgM antibody, though other methods are also possible. IgG antibodies produced by such conversion from Kt-IgM-8 are therefore encompassed within the scope of the present disclosure. Humanized versions of the Kt-IgM-8 antibody are also encompassed within the present disclosure. Humanized antibodies are antibodies from non-human species whose protein sequences have been modified to increase their similarity to antibody variants produced naturally in humans. Humanized antibodies can be produced through methods known in the art, such as by utilizing insect-cell expression systems. Furthermore, antibody chimeras—that is, chimeric versions of the Kt-IgM-8 antibody—such as half-human half-murine antibodies, are also encompassed within the present disclosure. One non-limiting example of a chimeric antibody includes a mouse Fab spliced to a human Fc.

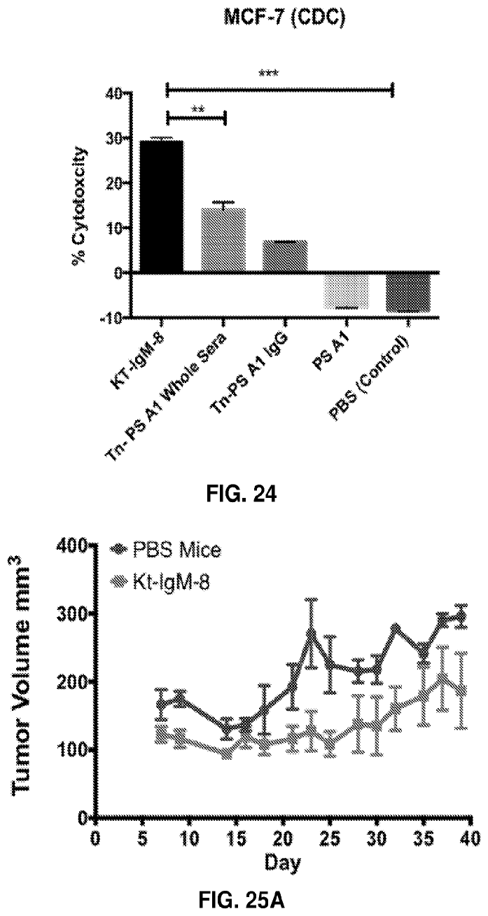

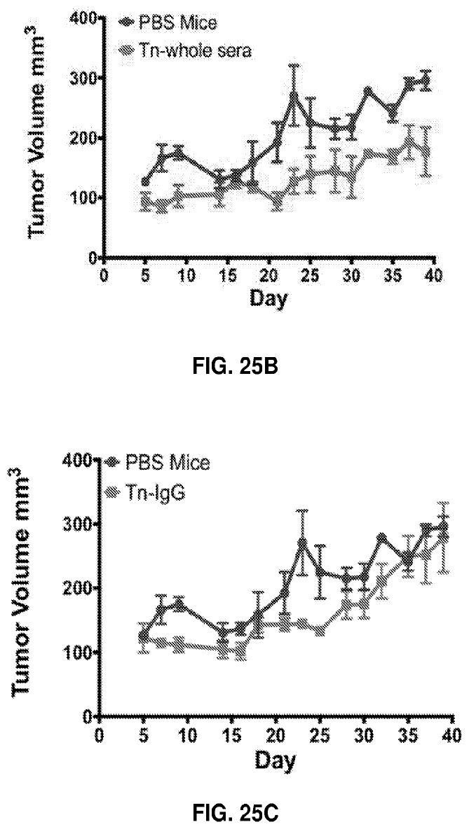

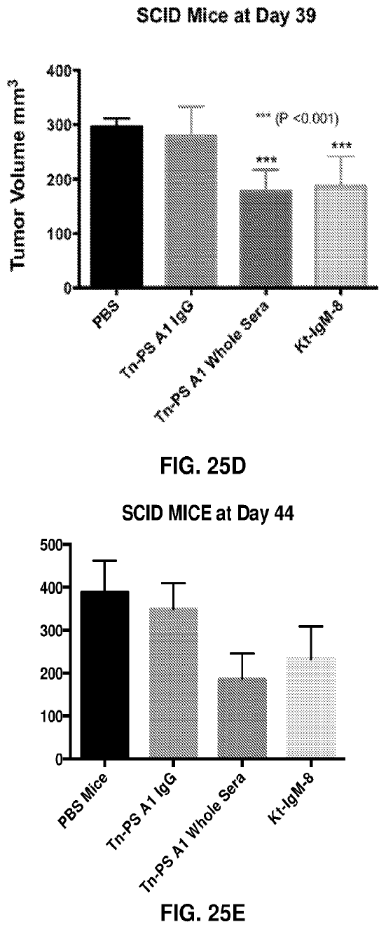

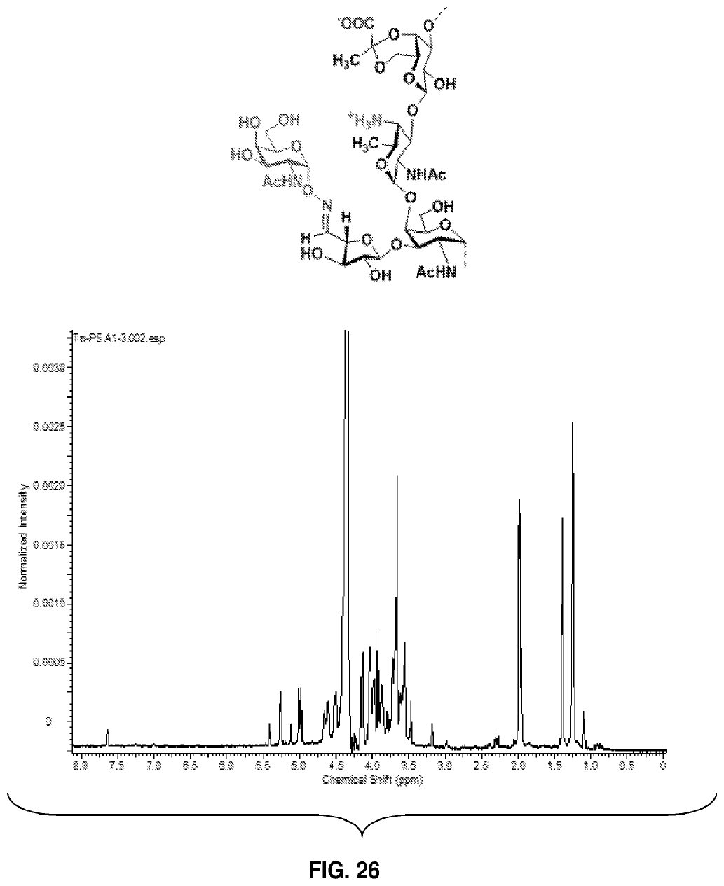

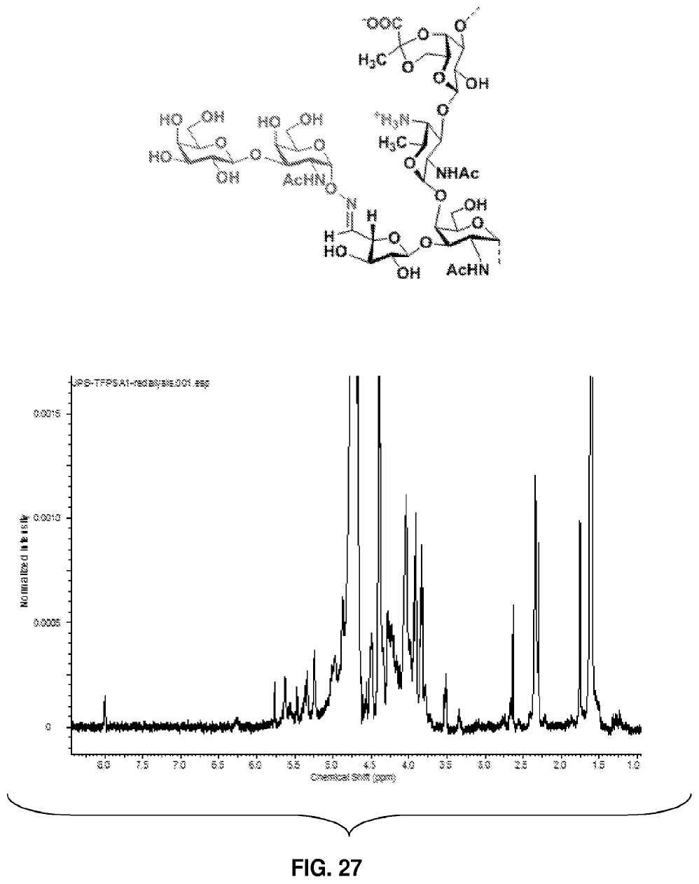

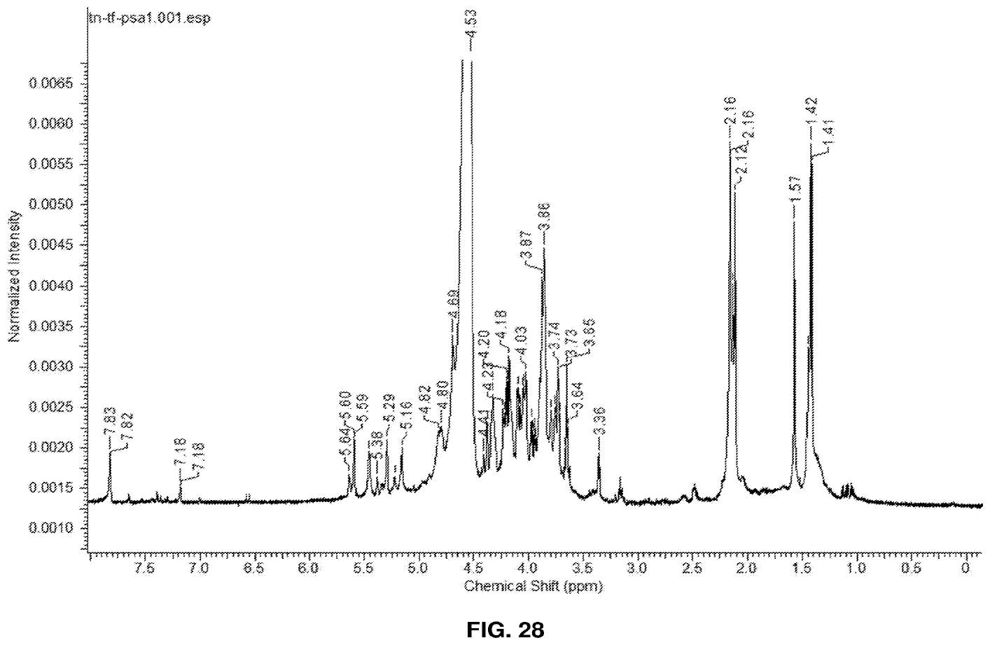

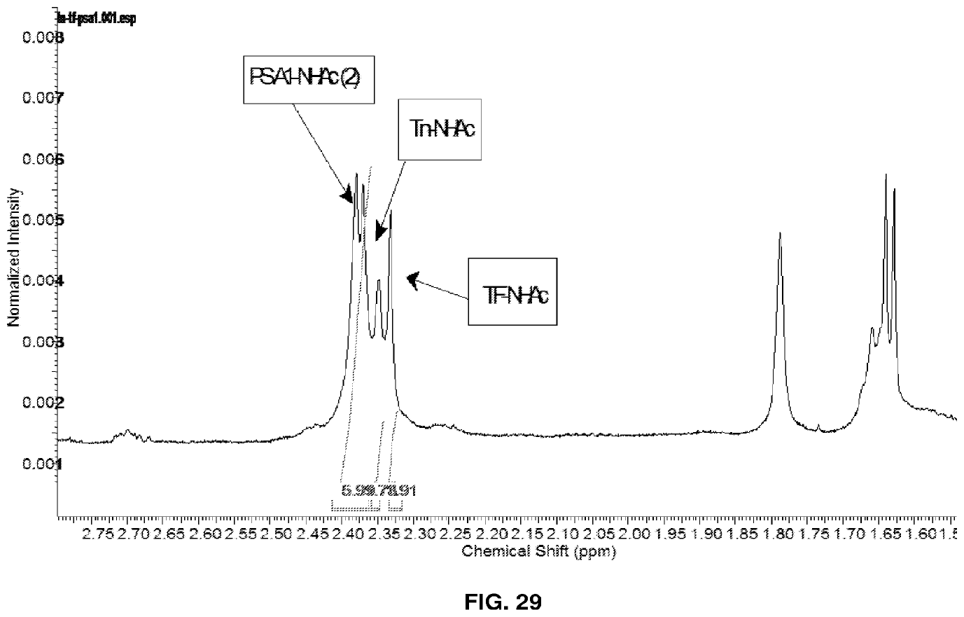

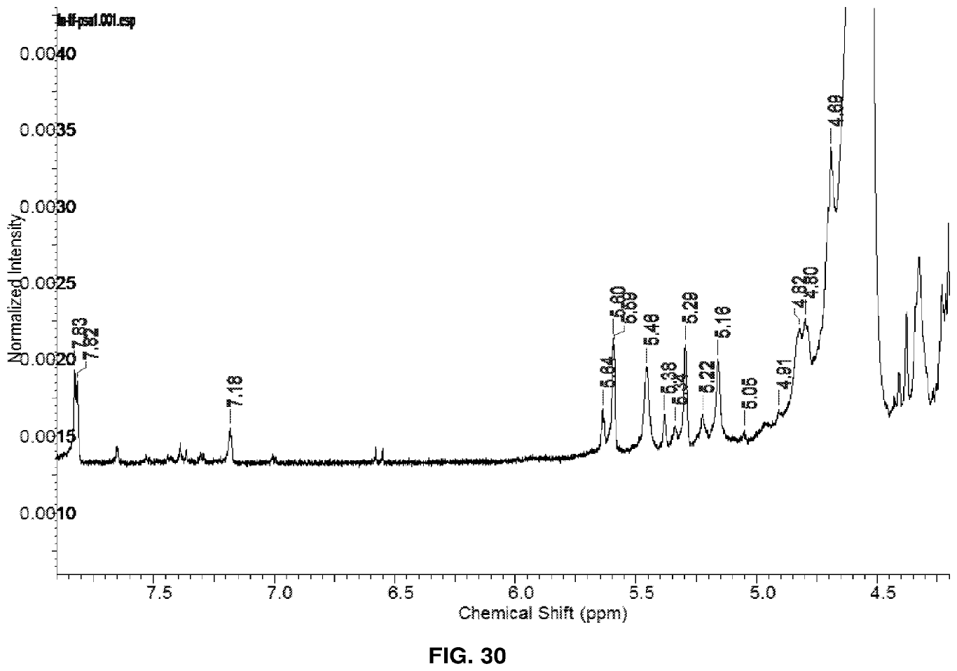

As noted above, Kt-IgM-8 is an IgM antibody. Monoclonal IgMs have often been demonstrated to bind oligomers better than their IgG counterparts through higher avidity. IgM antibodies have demonstrated recognition of carbohydrate antigens greater than their IgG counterparts through higher avidity. Since TACAs are present on almost all cancers, having an immunotherapy that can recognize specific glycosides is an efficient strategy against cancer. More importantly, IgM antibodies have been shown to be effective at mediating complement directed killing of tumor cells. Thus, using a specific mIgM for aberrant glycosylation patterns such as those found on tumor cells is a unique therapeutic approach to targeting carbohydrates and malignant cells. The advantage of this approach is specifically targeting the carbohydrate portions of TACAs using a mIgM that is specific for the surface modification of tumors. Because TACAs are present on the surface of most tumors, having a mAb that can recognize specific glycosides is a useful therapeutic strategy in light of most mAbs targeting proteins that are concealed beneath the glycocalyx. Since mIgM antibodies described herein have been shown to be efficient at mediating complement directed (CDC) (FIG. 24) killing of tumor cells, this approach offers a useful strategy for recognizing known human tumor cell lines as noted by FACS. Using SCID (Severe Combined Immunodeficiency) mice xenografted with a breast cancer cell line (MCF-7), a reduction in tumor volume by ˜30% compared to control mice (PBS) was demonstrated (FIG. 25D).

The mAbs described herein are generated from entirely carbohydrate immunogen constructs, which are administered into an animal to provoke an immune response. Then, a hybridoma method is used to produce large numbers of monoclonal antibodies. For example, B cells are harvested from the animal and fused with B cell cancer cells to produce hybridoma cells that produce the antibodies. In the examples herein, the Tn antigen was conjugated to the zwitterionic polysaccharide PS A1, via a linkerless strategy, to create an entirely carbohydrate vaccine/immunotherapy (Tn-PS A1). The rationale behind a carbohydrate-based construct is to fine-tune the immune response to target carbohydrates exclusively, a long outstanding problem in immunity. To take advantage of the unique immune response from this construct, monoclonal antibodies that recognized the Tn antigen exclusively and not a heterogeneous combination of carbohydrate/peptide moieties were generated. The mAb generated from this particular example is Kt-IgM-8. One difference in this approach from traditional methods (i.e., Tn conjugated to immunogenic proteins such as BSA, TT, or KLH) is epitopic suppression of the carbohydrate moiety due to the overwhelming immunogenicity of proteins of peptide immunogens. Additionally, mAbs generated from Tn containing peptides/proteins often recognize the peptide portion better than the glycan. However, the mAbs generated from an entirely carbohydrate moiety specifically recognize Tn without assistance from peptide binding.

Zwitterionic polysaccharides (ZPSs) are compounds having both positive and negative charges on adjacent monosaccharide units, and are known as being T cell stimulatory independent of peptides, proteins, or lipids. ZPSs are alternatives to protein carriers for vaccines and immunotherapeutics, and have been shown to initiate a CD4+ T cell response. ZPSs are processed by antigen presenting cells (APCs) and presented as MHCII-ZPS complexes on the surface for α/β-TCR recognition of CD4+ T-cells that can promote immunoglobulin class switching from IgM to IgG. Using ZPSs as immunogenic carriers for TACAs can augment the immune response by generating entirely carbohydrate specific antibodies. As one example, PS A1 from Bacteroides fragilis (ATCC 25285/NTTC 9343) is a naturally occurring polysaccharide that can generate a CD4+ T-cell mediated immune response. Due to this unique feature, PS A1 is useable as a carrier for tumor associated carbohydrate antigen (TACA) Thomsen-nouveau (Tn-α-2-NAc-D-galactose).

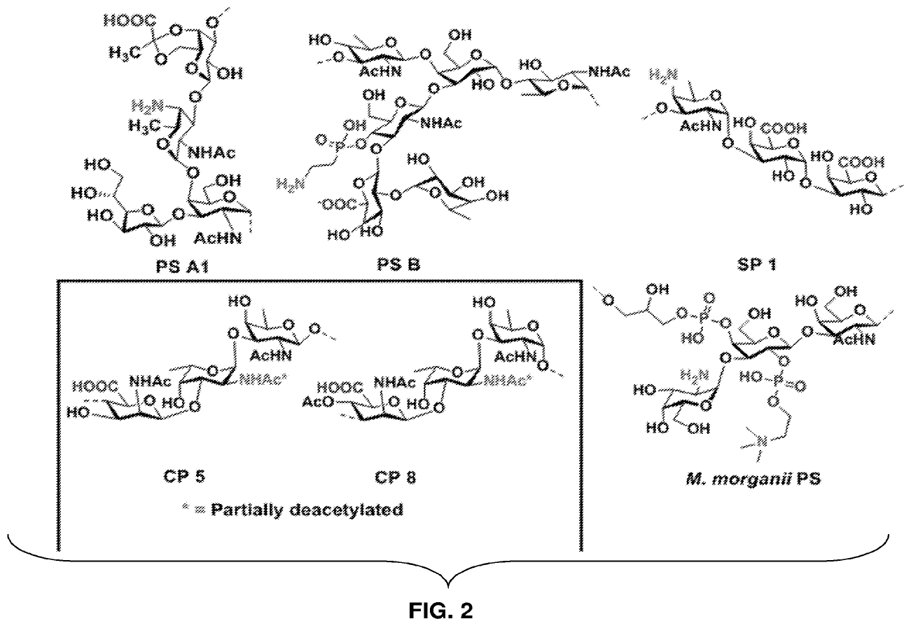

In accordance with the present disclosure, TACAs are conjugated to ZPSs to produce entirely carbohydrate immunogens, for example to raise antibodies. Suitable ZPSs include, but are not limited to, ZPSs isolated from pathogenic bacteria, such as PS A1 from Bacteroides fragilis, PS B from Bacteroides fragilis, SP1 from Streptococcus pneumonia, CP5 from Staphylococcus aureus (CP5 has partial deactylation of NHAc on L-FucNAc), CP8 from Staphylococcus aureus (CP8 has partial deacetylation of NHAc on L-FucNAc), and PS from Morganella morganii. (FIG. 2.) PS A1 contains a repeating zwitterionic tetrasaccharide unit that contains a [3-2,4-dideoxy-4-amino-D-N-acetylfucose (1-4), D-N-acetylgalactosamine(1-3), D-galactopyranose(1-3), D-galactofuranose] with a 4,6-pyruvate acetal. PS A1 has been shown to adapt an alpha helical character, which is a common characteristic of proteins and can be determined by circular dichroism. PS A1 can also be recognized by the immune system and processed via MHC II, which was once thought to exclusively bind peptide fragments. PS B is a high molecular weight ZPS with repeating sugars: β-D-QuiNac (1→4), α-D→Gal (1-4), α-L-QuiNAc (1→3), and branched from 3′-galactose is β-D-GlcNAc (1→3), α-D-GalA(1→3), and α-L-Fucp(1→2). In addition, a bacterial exclusive 2-aminoethyl phosphonate moiety is substituted on the 4′ position of β-D-GlcNAc. Other zwitterionic polysaccharides, such as SP1, have been noted to induce CD4+ and CD8+CD28− T cells in C57BL/6 mice. CP5 and CP8 both induce intra-abdominal abscesses, which signifies a potent T cell response. The ZPS from M. morganii has also been shown to interact with MHC II, and stimulate T cell activation.

Suitable TACAs include, but are not limited to, the O-linked mucins Tn, TF (Galβ1→3GalNAcα1→Ser/Thr), and STn; the Lewis antigens Le^{y}, Le^{x}, SLe^{x}, Le^{a}, SLe^{a}, and Le^{b}; the globosides Globo H and SSEA4; the gangliosides GD2, GD3, and GM3; and conjugates of two or more thereof. As one non-limiting example, conjugation of the antigen to the ZPS can be accomplished via oxime bond formation. However, other orthogonal conjugation chemistries, such as click chemistry forming triazoles, are possible.

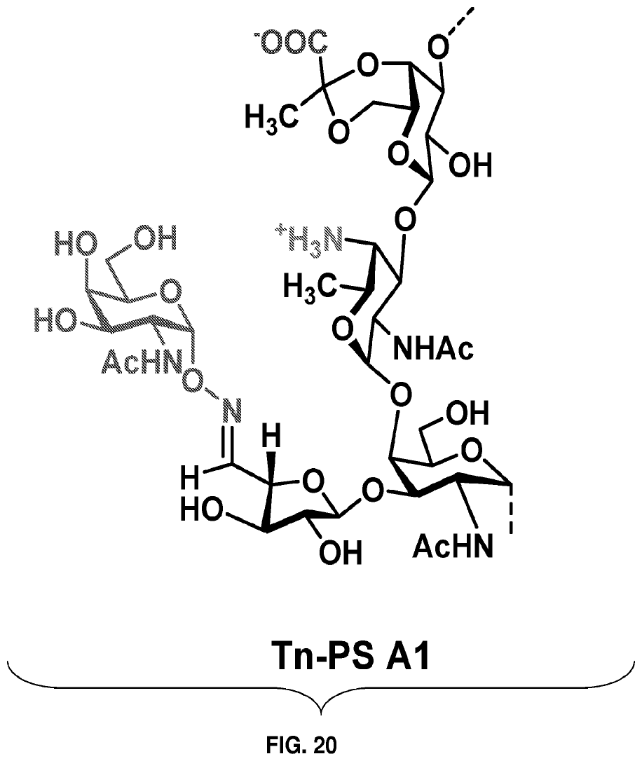

In some embodiments, the Thomsen-nouveau (Tn) antigen (α-D-GalNAc) is conjugated to PS A1, creating an entirely carbohydrate vaccine or immunotherapeutic (Tn-PS A1). Tn-PS A1 (GalNAc-PS A1) (FIG. 20) is an entirely carbohydrate-based construct that is useful as an alternative to TACA-protein conjugates. The TACA-PS A1 immunogen uses a zwitterionic polysaccharide, PS A1, from B. fragilis, which can be oxidized for oxime formation by aminooxy-TACAs conjugations. This structure stimulates an anti-tumor response through the induction of CD4+ T cells and production of various cytokines: IL-10, IL-17A, IL-4, and IL-2, and it thus leads to carbohydrate-specific IgG and IgM antibodies. This method for tumor treatment is a valuable tool in treating/preventing cancers. It can also be harnessed to produce monoclonal antibodies (mAb) due to the selective and specific anti-carbohydrate immune response. The Tn antigen is a valuable target due to its high expression on tumor cells (˜80-90% of all tumors) and synthetic accessibility for biological conjugations.

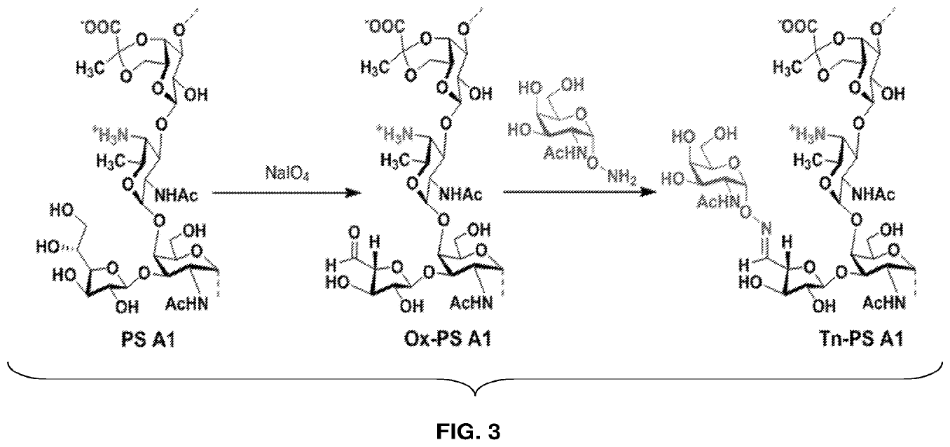

Tn can be conjugated to PS A1 by, for instance, synthesizing the Tn-aminooxy sugar to form an oxime linkage with oxidized PS A1 (FIG. 3). The oxime linkage is favored over hydrazone and imine linkages due to the hydrolytic stability of the oxime linkage at physiological pH. Hydrolysis of the hydrazone is favored over the oxime due to the lower electronegativity of the nitrogen, which is more readily protonated compared to the oxygen of the oxime. Accordingly, the oxime linkage provides stability even in acidic environments (pH 3-4), which TACA-PS A1 encounters in the lysosomes after antigen uptake, making the oxime linkage more suitable for a vaccine composition. Alternatively, if PS A1 is conjugated with TACAs containing either hydrazine or hydrazides functional groups, the hydrazone linkage is more susceptible to hydrolysis and will decrease TACA density on PS A1, which may decrease the immune response to the TACA hapten.

Utilizing a synthetically prepared Tn-hydroxyl amine conjugated to oxidized galactofuranose, the formation of an oxime bond provides a unique entirely carbohydrate immunogen without the need of bulky immunogenic linkers. The advantage of this structure is to emphasize the immune response on 0-linked carbohydrates by the linker-free oxime ligation and not on O-linked glycopeptides. For example, when examining mAbs towards glycopeptides, binding tends to be influenced by the original peptide sequence and is thus not glycan-specific. Traditional methods for mAb production have used naturally occurring TACAs (i.e., cancer cells and glycosylated proteins), and have led to many non-specific and commercially available mAbs such as B1.1 and Tn218 (IgM). These two mAbs were generated from ovine submaxillary mucin and screened for Tn binding. The complication that is associated with glycoproteins is carrier-induced epitopic suppression that is due to the greater immunogenicity of the protein carrier. This leads to mAbs dependent upon natural linkages. Therefore, most mAbs generated from glycopeptides/proteins/linkers will have a varying sensitivity towards the peptide/linker portion, which is one of the reasons an entirely carbohydrate immunogen was used to assist in mAb development. The use of Tn-PS A1 to generate mAbs produces superior antibodies specifically for glycosides, which leads to sufficient anti-tumor responses.

In some embodiments, the Thomsen Friedenreich (TF) antigen (α-D-Gal-(1,3)-β-D-GalNAc) is conjugated to PS B or PS A1. As described in the examples herein, TF-PS B conjugate was immunized in Jax C57BL/6 mice to produce both IgG and IgM antibody responses specific for the TF antigen. Enhanced binding to the TF-containing MCF-7 breast cancer cell line was shown by fluorescence activated cell sorting (FACS). Additionally, TF-PS A1 elicits similar augmented immune responses to the TF antigen, which enables in vitro cytotoxicity of tumor cells. In comparison to Tn-PS A1, both the TF-PS B and TF-PS A1 immunogens generate substantially decreased IgG antibody production, which is a main component of the mechanism for tumor elimination. However, the IgG immune responses to the TF antigen can be increased by using a bivalent PS A1 construct.

In some embodiments, the sialyl Tn antigen is conjugated to PS A1, producing a construct having the following Formula I:

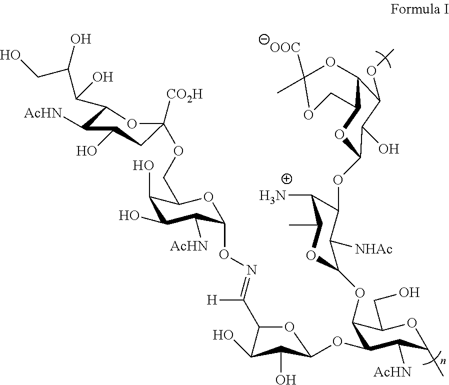

In some embodiments, the construct is a bivalent immunogen, such as a Tn-TF-PS A1 bivalent immunogen. This immunogen significantly increases immunogenicity of the TF antigen. This additive “Tn adjuvanting effect” also generates enhanced pIgG binding to tumor cell lines MCF-7 and OVCAR-5 in FACS analysis and in a complement dependent cytotoxicity (CDC) assay monitoring lactate dehydrogenase (LDH) release from these tumor cells. The results from a CDC assay demonstrated increased tumor cell lysis from Tn-TF-PS A1 sera compared to sera from monovalent vaccines Tn-PS A1 and TF-PS A1. Furthermore, a macrophage galactose lectin 2 (MGL2) assay was used, in conjunction with designed biotinylated probes, to study binding interactions of Tn and TF conjugated to PS A1 vaccine constructs. The results showed that, in the case of the TF antigen, when a unimolecular bivalent Tn-TF-PS A1 immunogen was used, immunogenicity of the TF antigen was increased 50 times over a monovalent TF-PS A1 construct and resulted in a more potent and selective immune response. This not only validates a MGL2 targeted vaccine design, but also indicates that incorporating a Tn antigen can influence other peptide, protein, or lipid vaccine designs. To show the usefulness of unimolecular bivalent immunogens, this model was adapted to a Globo H-PS A1 construct consisting of Globo H and Tn. Similar to the biological results of Tn-TF-PS A1, the Tn-Globo H-PS A1 immunogen produced a robust IgG immune response with cytotoxicity towards both MCF-7 and HCT-116 cancer cells.

The entirely carbohydrate constructs provided herein are carbohydrate immunogen compositions that can be used for purposes other than producing monoclonal antibodies, and can assist in both tumor binding and killing. For example, these constructs can be used as vaccines to treat or prevent cancers. Such vaccine compositions can include Formula I, or the following Formula II:

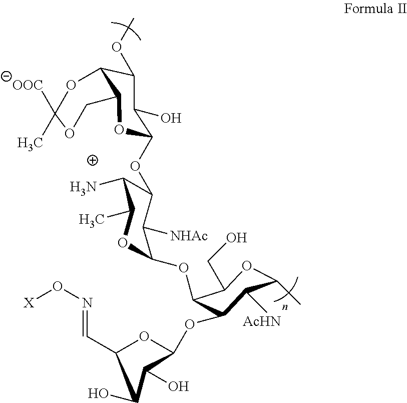

where X is Tn, TF, Tn-TF, Gb3, Globo H, or conjugates thereof. Either Formula I or Formula II can be produced by oxime bond formation. Alternatively, these entirely carbohydrate immunogens can be produced by reductive amination. In any event, the generation of monoclonal antibodies from these constructs can provide entirely carbohydrate recognition without the influence from peptides or proteins.

The detection, or quantitative determination, or both, of analytes based upon reactions with immunological reagents has gained considerable importance in the field of medical testing. These methods commonly involve contacting a sample suspected of containing the analyte with a material which exhibits specific immunologic reactivity with the analyte, for example, an antibody directed to an epitope present on the analyte. If the analyte is present in the sample, it specifically conjugates with the antibody to form a complex. A wide range of developer or reporter mechanisms are known for indicating whether the conjugation reaction occurs. Such methods are especially important with monoclonal antibodies because of the unique specificity for the analytes with which they conjugate. These methods can be practiced through a range of test devices, test kits, and the like, for instance with devices that utilize “flow through” membrane procedures for rapid testing (e.g., 5-10 minutes). Thus, provided herein are test devices, test kits, or test strips which utilize the monoclonal antibodies described herein (produced from entiretly carbohydrate immunogens) to test for the presence of an analyte, such as a TACA, in a sample. The antibody is generally labeled, for example, with a radioactive isotope, fluorophore, chromophore, or a ligand which can be used with an enzyme that catalyzes a chemical reaction which produces a detectable product that can be further amplified in a secondary reaction. Suitable labeling agents generally include, but are not limited to: enzymes, such as peroxidase, alkaline phosphatase, β-D-galactosidase, glucose oxidase, glucose-6-phosphate dehydrogenase, alcohol dehydrogenase, malate dehydrogenase, penicillinase, catalase, apo-glucose oxidase, urease, luciferase or acetylcholinesterase; fluorescent materials, such as fluorescein isothiocyanate, phycobiliprotein, chelating compounds of rare-earth metals, dansyl chloride, or tetramethylrhodamine isothiocyanate; chemiluminescent materials; biotin; avidin; or radioactive isotopes.

Further provided are pharmaceutical compositions containing monoclonal antibodies described herein. Pharmaceutical compositions of the present disclosure comprise an effective amount of a monoclonal antibody, and/or additional agents, dissolved or dispersed in a pharmaceutically acceptable carrier. The phrases “pharmaceutical” or “pharmacologically acceptable” refer to molecular entities and compositions that produce no adverse, allergic, or other untoward reaction when administered to an animal, such as, for example, a human. The preparation of a pharmaceutical composition that contains at least one compound or additional active ingredient will be known to those of skill in the art in light of the present disclosure, as exemplified by Remington's Pharmaceutical Sciences, 2003, incorporated herein by reference. Moreover, for animal (e.g., human) administration, it is understood that preparations should meet sterility, pyrogenicity, general safety, and purity standards as required by FDA Office of Biological Standards.

A composition disclosed herein may comprise different types of carriers depending on whether it is to be administered in solid, liquid or aerosol form, and whether it need to be sterile for such routes of administration as injection. Compositions disclosed herein can be administered intravenously, intradermally, transdermally, intrathecally, intraarterially, intraperitoneally, intranasally, intravaginally, intrarectally, intraosseously, periprosthetically, topically, intramuscularly, subcutaneously, mucosally, intraosseosly, periprosthetically, in utero, orally, topically, locally, via inhalation (e.g., aerosol inhalation), by injection, by infusion, by continuous infusion, by localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 2003, incorporated herein by reference).

The actual dosage amount of a composition disclosed herein administered to an animal or human patient can be determined by physical and physiological factors such as body weight, severity of condition, the type of disease being treated, previous or concurrent therapeutic interventions, idiopathy of the patient, and the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of a monoclonal antibody. In other embodiments, an active compound may comprise between about 2% to about 75% of the weight of the unit, or between about 25% to about 60%, for example, and any range derivable therein. Naturally, the amount of monoclonal antibody in each therapeutically useful composition may be prepared is such a way that a suitable dosage will be obtained in any given unit dose of the antibody. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In certain embodiments, a composition herein and/or additional agent is formulated to be administered via an alimentary route Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsules, they may be compressed into tablets, or they may be incorporated directly with the food of the diet.

In further embodiments, a composition described herein may be administered via a parenteral route. As used herein, the term “parenteral” includes routes that bypass the alimentary tract. Specifically, the pharmaceutical compositions disclosed herein may be administered, for example but not limited to, intravenously, intradermally, intramuscularly, intraarterially, intrathecally, subcutaneous, or intraperitoneally.

Solutions of the compositions disclosed herein as free bases or pharmacologically acceptable salts may be prepared in water suitably mixed with a surfactant, such as hydroxypropylcellulose. Dispersions may also be prepared in glycerol, liquid polyethylene glycols, and mixtures thereof, and in oils. Under ordinary conditions of storage and use, these preparations may contain a preservative to prevent the growth of microorganisms. The pharmaceutical forms suitable for injectable use include sterile aqueous solutions or dispersions and sterile powders for the extemporaneous preparation of sterile injectable solutions or dispersions. In some cases, the form should be sterile and should be fluid to the extent that easy injectability exists. It should be stable under the conditions of manufacture and storage and should be preserved against the contaminating action of microorganisms, such as bacteria and fungi. The carrier can be a solvent or dispersion medium containing, for example, water, ethanol, polyol (i.e., glycerol, propylene glycol, liquid polyethylene glycol, and the like), suitable mixtures thereof, and/or vegetable oils. Proper fluidity may be maintained, for example, by the use of a coating, such as lecithin, by the maintenance of the required particle size in the case of dispersion, and/or by the use of surfactants. The prevention of the action of microorganisms can be brought about by various antibacterial and antifungal agents, such as, but not limited to, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption such as, for example, aluminum monostearate or gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in 1 mL of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

Sterile injectable solutions are prepared by incorporating the compositions in the required amount in the appropriate solvent with various other ingredients enumerated above, as required, followed by filtered sterilization. Generally, dispersions are prepared by incorporating the various sterilized compositions into a sterile vehicle which contains the basic dispersion medium and the required other ingredients from those enumerated above. In the case of sterile powders for the preparation of sterile injectable solutions, some methods of preparation are vacuum-drying and freeze-drying techniques which yield a powder of the active ingredient plus any additional desired ingredient from a previously sterile-filtered solution thereof. A powdered composition is combined with a liquid carrier such as, but not limited to, water or a saline solution, with or without a stabilizing agent.

In other embodiments, the compositions may be formulated for administration via various miscellaneous routes, for example, topical (i.e., transdermal) administration, mucosal administration (intranasal, vaginal, etc.) and/or via inhalation.

Pharmaceutical compositions for topical administration may include the compositions formulated for a medicated application such as an ointment, paste, cream, or powder. Ointments include all oleaginous, adsorption, emulsion, and water-soluble based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones, and luarocapram. Possible bases for compositions for topical application include polyethylene glycol, lanolin, cold cream, and petrolatum, as well as any other suitable absorption, emulsion, or water-soluble ointment base. Topical preparations may also include emulsifiers, gelling agents, and antimicrobial preservatives as necessary to preserve the composition and provide for a homogenous mixture. Transdermal administration of the compositions may also comprise the use of a “patch.” For example, the patch may supply one or more compositions at a predetermined rate and in a continuous manner over a fixed period of time.

In certain embodiments, the compositions may be delivered by eye drops, intranasal sprays, inhalation, and/or other aerosol delivery vehicles. Methods for delivering compositions directly to the lungs via nasal aerosol sprays has been described in U.S. Pat. Nos. 5,756,353 and 5,804,212 (each specifically incorporated herein by reference in their entirety). Likewise, the delivery of drugs using intranasal microparticle resins and lysophosphatidyl-glycerol compounds (U.S. Pat. No. 5,725,871, specifically incorporated herein by reference in its entirety) are also well-known in the pharmaceutical arts and could be employed to deliver the compositions described herein. Likewise, transmucosal drug delivery in the form of a polytetrafluoroetheylene support matrix is described in U.S. Pat. No. 5,780,045 (specifically incorporated herein by reference in its entirety), and could be employed to deliver the compositions described herein.

It is further envisioned the compositions disclosed herein may be delivered via an aerosol. The term aerosol refers to a colloidal system of finely divided solid or liquid particles dispersed in a liquefied or pressurized gas propellant. The typical aerosol for inhalation consists of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight, and the severity and response of the symptoms.

In particular embodiments, the compositions described herein are useful for treating, preventing, or ameliorating a cancer, such as breast cancer.

Furthermore, the compositions herein can be used in combination therapies. That is, the compositions can be administered concurrently with, prior to, or subsequent to one or more other desired therapeutic or medical procedures or drugs. The particular combination of therapies and procedures in the combination regimen will take into account compatibility of the therapies and/or procedures and the desired therapeutic effect to be achieved. Combination therapies include sequential, simultaneous, and separate administration of the active ingredients in a way that the therapeutic effects of the first administered procedure or drug is not entirely disappeared when the subsequent procedure or drug is administered.

By way of a non-limiting example of a combination therapy, a composition herein can be administered in combination with one or more suitable chemotherapeutic agents including, but not limited to: platinum coordination compounds; taxane compounds; topoisomerase I inhibitors, such as camptothecin compounds; topoisomerase II inhibitors, such as anti-tumor podophyllotoxin derivatives; anti-tumor vinca alkaloids; anti-tumor nucleoside derivatives; alkylating agents; anti-tumor anthracycline derivatives; HER2 antibodies; estrogen receptor antagonists or selective estrogen receptor modulators; aromatase inhibitors; differentiating agents, such as retinoids, and retinoic acid metabolism blocking agents (RAMBA); DNA methyl transferase inhibitors; kinase inhibitors; farnesyltransferase inhibitors; HDAC inhibitors; other inhibitors of the ubiquitin-proteasome pathway; or combinations thereof.

Further provided is a method of determining health insurance reimbursement or payments, the method comprising denying coverage or reimbursement for a treatment, where the treatment comprises a monoclonal antibody, or a vaccine comprising an entirely carbohydrate immunogen, described herein.

### EXAMPLES

### Example 1—STn-PS A1 as an Entirely Carbohydrate Immunogen: Synthesis and Immunological Evaluation

Sialyl Tn (STn) is a tumor associated carbohydrate antigen (TACA) that is overexpressed in a variety of carcinomas such as breast, ovarian, and colon cancer. In normal tissue, STn is not detectable, which is important for opportunities in developing cancer immunotherapies. An entirely carbohydrate, semi-synthetic STn-PS A1 conjugate was prepared and evaluated in C57BL/6 mice. STn-PS A1 was combined with commercially available monophosphoryl lipid A (MPL)-based adjuvant and after immunization, ELISA indicated a strong immune response for inducing both anti-STn IgM/IgG antibodies. The specificity of these antibodies was concomitantly investigated using FACS analysis and the results indicated excellent cell surface binding events to STn-expressing cancer cell lines MCF-7 and OVCAR-5. Most importantly, the raised antibodies conferred complement-dependent cellular cytotoxicity against MCF-7 and OVCAR-5 cells.

The STn antigen (Neu5Acα2-6GalNAcα-O-Ser/Thr) is an O-linked mucin TACA that is overexpressed in human carcinomas and negligible in fetal and adult tissues. In cancer cells, the biosynthesis of STn is catalyzed by sialyltransferase ST6GalNAc I, which outcompetes other O-glycan elongating glycosyltransferases and promotes the generation of truncated sialylated O-glycans on cancer cell surfaces. Detection of STn is associated with various types of cancers, such as breast and ovarian, and high levels of STn correlate with a poor prognosis for patients. Therefore, STn is a relevant target for tumor immunotherapy. In the past few decades, many synthetic chemists, immunologists, and vaccinologists alike have been dedicated to the development of effective cancer vaccines that target STn or STn-related mucins.

Utilization of the zwitterionic polysaccharide PS A1 as a “carrier” for a Thomsen-nouveau (Tn)-PS A1 entirely carbohydrate immunogen has been demonstrated. This construct invoked a T cell dependent immune response capable of binding the Tn antigen and less concern of possible epitope suppression to the carbohydrate antigens. Provided in this Example is a more synthetically challenged conjugation, STn-PS A1, and more detailed immunological studies of STn-PS A1 as an entirely carbohydrate vaccine construct in combatting breast and ovarian tumors.

Results and Discussion

Synthesis of Aminooxy STn and Alpha-Selective Sialylation

Challenges in the chemical syntheses of sialyl-containing oligosaccharides are stereo-selective sialylation and rate enhancement. In order to improve reactivity and selectivity of α-sialylation, previous attempts have focused on the development of activating groups at the anomeric position, installation of auxiliary groups at C-1 and C-3, incorporation of strong electron-withdrawing groups on the nitrogen atom at C-5, and the use of stereo-directing nitrile solvents. Utilizing a 4N 5O trans-fused oxazolidinone moiety at the C-4 and C-5 positions has led to excellent alpha selectivity because of the strong electron-withdrawing nature and trans orientation of the oxazolidinone group. These effects created a dipolar moment that greatly diminished the anomeric effect, which subsequently led to a new equilibrium favoring the formation of α-sialyl glycosides. Phosphate esters have been used as anomeric-leaving groups in many glycosylation reactions, and benefits include augmented reactivity as well as facile activation, especially when compared to the widely applied thiol-leaving group. The combination of oxazolidinones and phosphates in sialylation reactions leads to highly alpha-selective and highly reactive sialyl donors, which have been proven to be an optimized solution for O-, S-, and C-sialylation.

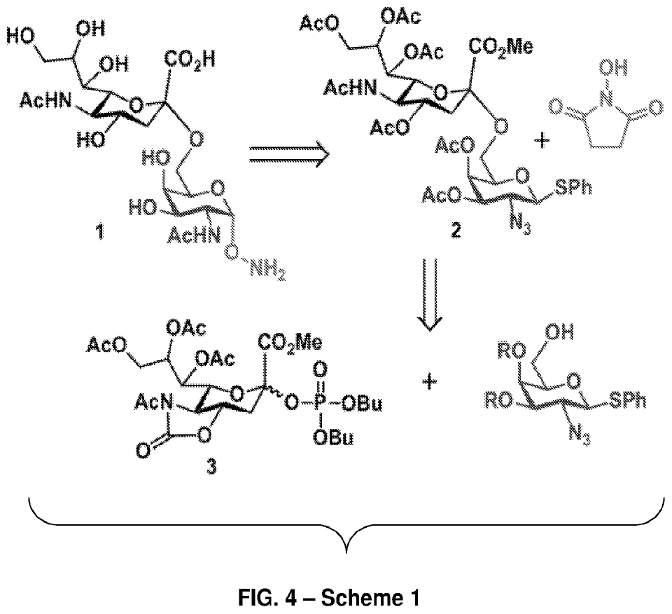

Based on the above, the synthesis of α-aminooxy STn (1) was conducted as shown in Scheme 1 (FIG. 4), and includes a key stereochemical transformation that is highly alpha-selective between sialyl donor 3 and a suitably protected 2-azido-galactose acceptor. The resulting disaccharide can undergo a simple protecting group manipulation that can readily yield compound 2. Introduction of the N-hydroxysuccinimide at the reducing end of 2 allows for the desired aminooxy disaccharide. The sialyl carboxylic methyl ester can be easily and selectively removed prior to the removal of the N-succinimidyl and acetyl groups, which results in the desired compound 1.

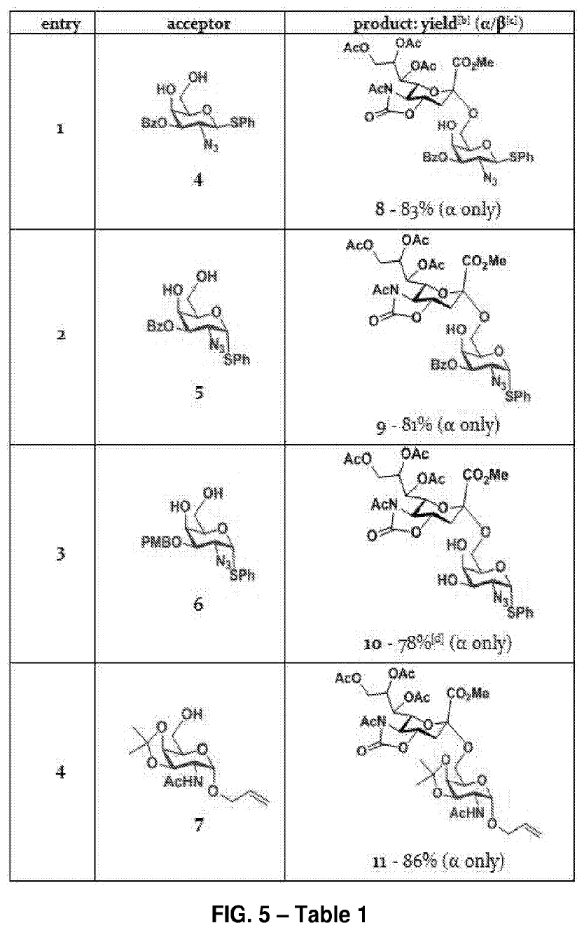

Based on this glycosylation strategy, early stage synthetic efforts focused on the investigation of the optimized acceptors for sialylation as shown in Table 1 (FIG. 5). Three thiol-galactopyranoside acceptors 4, 6, and 8 that included free hydroxyls at the 4,6 positions were designed and tested. The reactions led to excellent yields and quantitative alpha selectivity as observed in ¹H NMR analysis of the unpurified disaccharide.

As noted in Table 1 (FIG. 5); entries 1 and 2, the α-/β-acceptors led to good yields and α-selectivity of compounds 8 and 9. In entry 3, an interesting result was observed. After 30 minutes, TLC analysis indicated complete consumption of the sialyl donor and formation of two products. Without wishing to be bound by theory, it is believed that this was not an alpha/beta mixture, but rather a product of partial deprotection of the acid-sensitive p-methoxybenzyl (PMB) protecting group at the C-3 position. Instead of quenching the reaction, the temperature was raised to 0° C. to pursue complete in situ deprotection of the PMB group. After 45 minutes, quantitative removal of the PMB group was noted from TLC. Full characterization and analysis of isolated product 9 showed that this was not an alpha/beta mixture. In entry 4, a 2,3-protected allyl-galactopyranoside 7 was tested, and again only alpha glycosidic bond formation was observed, in 86% yield.

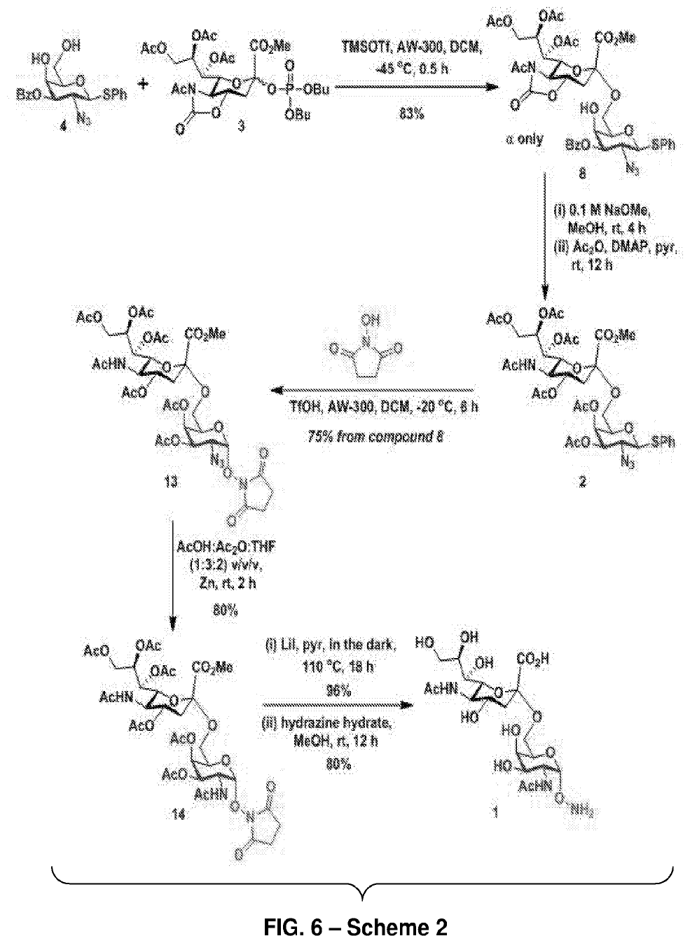

As shown in Scheme 2 (FIG. 6), the synthesis of α-aminooxy STn commenced from alpha selective sialylation. Compound 4 was then used as an acceptor for the subsequent glycosylation reaction with sialyl phosphate donor 3. The reaction, which proceeded smoothly in the presence of TMSOTf in DCM at −45° C., resulted in the exclusive α-configured disaccharide 5 in excellent yield. The oxazolidinone-protecting group and acetyl groups were removed using the Zemplén method, and the free hydroxyls were protected using acetic anhydride in pyridine and DMAP to afford dissaccharide 2. The thiol-donor sugar 2 was then activated using the NIS/TfOH reagent system followed by addition of N-hydroxysuccinimide to obtain the key intermediate 13. Compound 13 was obtained with exclusive alpha selectivity and in 75% isolated yield from 8. Utilizing a nonparticipating azido group at the C-2 position of D-galactose is important for the alpha selectivity. Compound 14 was then afforded by a facile transformation that commenced from the concomitant reduction and acetylation of the 2-azido group using zinc powder and acetic anhydride under acidic conditions. This one-pot reduction/protection reaction was followed by a chemo-selective Krapcho demethylation of the sialyl methyl ester by treating 14 with lithium iodide and pyridine under refluxing conditions. Finally, a global deprotection of the sugar-oxysuccinimide was carried out using hydrazine hydrate yielding aminooxy STn (1) as the desired final product.

The deprotection of the oxazolidinone (8→13) was performed before installation of the oxysuccinimide group. The purpose for this sequence was to avoid any possible conflicts between the oxysuccinimide and oxazolidinone in later-stage deprotection steps. In order to achieve selective removal of the oxazolidinone, sodium methoxide was used. However, since the oxysuccinimide group is base label, caution was taken in view of the embedded imide bond, which is known to immediately cleave and convert to an amide plus a methyl ester under conditions of sodium methoxide. Moreover, removal of the amide bond can be very challenging in such circumstances because a strong acidic environment and heat is required, which can compromise the stereo integrity of the disaccharide itself. Furthermore, the Krapcho demethylation of sialyl methyl ester should be conducted prior to that of oxysuccinimide installation. There are two predominant reasons for this sequence of reaction conditions: 1) the nucleophilic hydrazine can attack the sialyl methyl ester and convert it to the carboxamide, and 2) the Krapcho reaction is highly specific to methyl esters and therefore the succinimide group will stay intact. This strategy can be adapted to other carbohydrate syntheses, especially for those containing both sialyl and aminooxy moieties.

Aminooxy STn Links to PS A1 Via Oxime

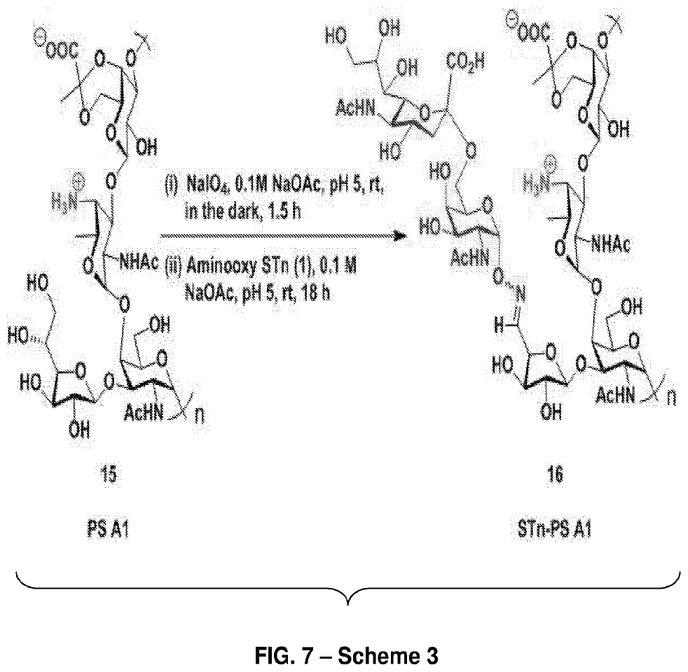

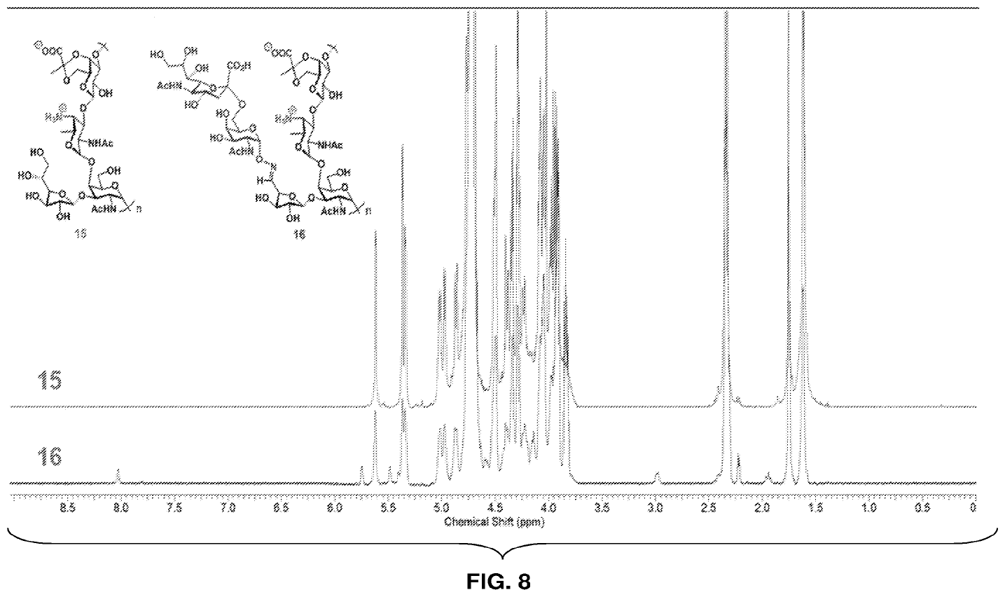

As shown in Scheme 3 (FIG. 7), aldehyde groups were introduced to PS A1 (15) by selectively oxidizing the terminal vicinal diols of the embedded galactofuranose residues with sodium periodate. Although another trans-diol presented on the galactofuranose residue, it is much less labile to periodate oxidation, thus only vicinal dial oxidation was observed. Aldehyde functionalized PS A1 was then conjugated with aminooxy STn (1) under slightly acidic conditions giving rise to the STn-PS A1 construct (16). The structure of STn-PS A1 (16) was confirmed with 1D and 2D NMR analyses. As a means to improve the resolution of spectra, all of the NMR experiments were performed at 60° C., as shown in FIG. 8, and then compared to naturally occurring PS A1. The peak at 8.02 ppm indicates the formation of an oxime bond, and anomeric protons on oxidized galactofuranose moieties appear at 5.48 ppm. COSY and 1D TOCSY experiments further confirmed the selectivity of periodate oxidation on vicinal diol, as well as the structure of the oxime-bearing-galactofuranose spin system. In FIG. 8, characteristic signals of the STn antigen were also identified as the anomeric proton of the GalNAc sugar was observed at 5.74 ppm. The equatorial and axial protons at C-3 of sialic acid were located at 2.99 ppm and 1.96 ppm, respectively. With the assistance of COSY and 1D TOCSY, GalNAc and the Neu5Ac spin systems of conjugated STn were delineated, and their structural features were noted to highly resemble that of monomeric aminooxy STn. Finally, the loading of STn to oxidized PS A1 was determined using two methods: 1)¹H NMR integration that allowed for determining the loading at 11%, and 2) use of the Svennerholm method, through which the loading was determined to be at 10%.

Immunological Studies: Antibody Response(s) Against the STn Antigen

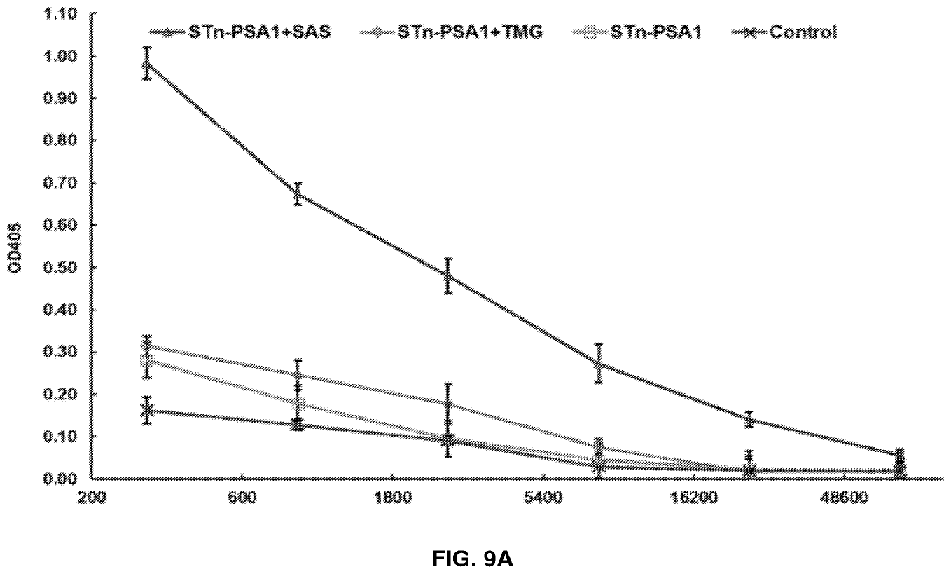

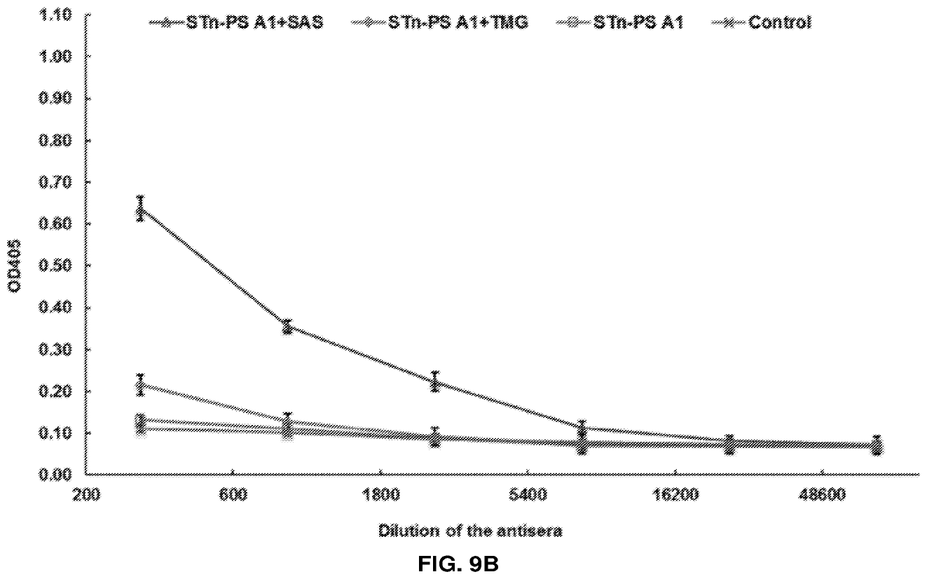

The utilization of natural STn-expressing mucins for serological assays can lead to more clinically relevant data compared to that of synthetic STn glycoprotein conjugates. It is known that the linkers of synthetic conjugates will exhibit a certain level of influence on antibody-antigen recognition events. Both ovine submaxillary mucin (OSM) and bovine submaxillary mucin (BSM) predominantly contain STn moieties, and have become the preferred choices for serological assays. In order to determine the specificity of the antibody induced by STn-PS A1 (16), sera from Jax C57BL/6 mice were collected and tested on BSM as shown in FIGS. 9A-9B. Sera obtained from mice immunized with STn-PS A1 plus Sigma Adjuvant System® (SAS) showed prominent binding events against BSM whereas sera from a group of mice injected with STn-PS A1 plus TiterMax® Gold (TMG) adjuvant produced moderate binding events of antibodies against BSM. The group of mice that was treated with only the STn-PS A1 (16) construct gave a moderate response to the natural STn antigen. Under these conditions, negligible IgG and IgM binding toward BSM was observed.

Based on the IgG and IgM ELISA results, there is clearly a benefit to utilizing suitable adjuvants. First, “adjuvant effects” can be beneficial for antigen-antibody binding events. The antibody titers of both adjuvanting groups, SAS and TMG, are multiple folds greater as observed in FIGS. 9A-9B. Furthermore, the choice of adjuvants can affect the outcome of antibody production. Monophosphoryl lipid A (MPL), which is the major component of SAS, preserves most of the immunostimulatory activity of lipid A with a significant decrease in toxicity. MPL is an agonist for TLR-4, which can increase the cellular immune response and is recommended in many types of mice immunizations. TiterMax® Gold (TMG), known as a “depot” adjuvant, is less toxic compared to SAS. However, the use of TMG can lead to inferior antibody production compared to MPL-containing vaccines. This is most likely a direct result of TMG's ability to protect the antigen from both dilution and rapid degradation and elimination by the host rather than target a specific receptor. There remains a lack of clear understanding about this strategy of covalently incorporating specific receptor-based adjuvants directly on vaccine constructs.

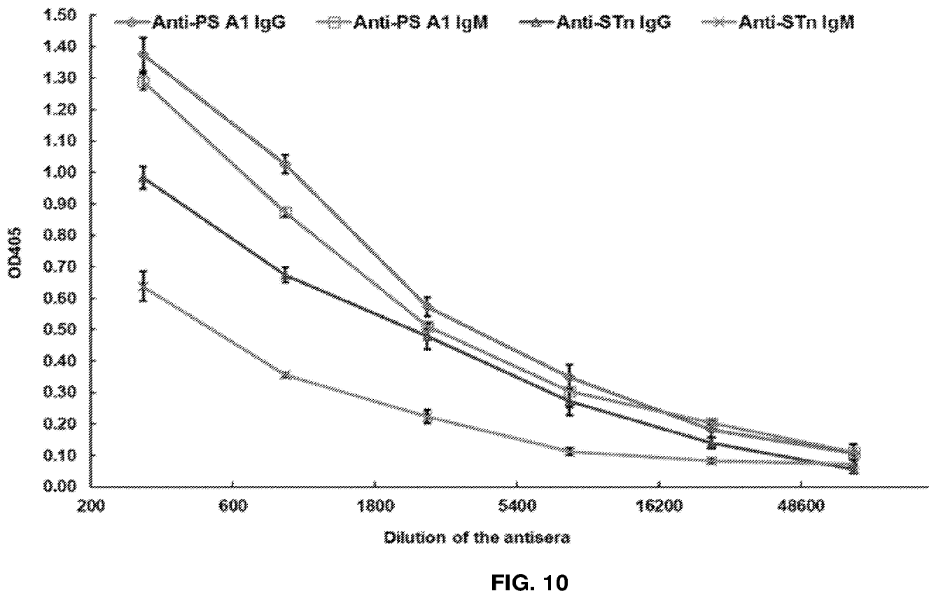

The safety of KLH protein has been shown previously. However, KLH is a very potent carrier protein. A very plausible concern of utilizing STn-KLH vaccine is epitope suppression, which is a result of overwhelming carrier-specific T cell response over that of conjugated antigens. Increased exposure of STn-KLH may lead to increased antibody response to KLH and diminished response to conjugated STn antigens. In order to properly evaluate the immunity of STn-PS A1 conjugate, it is necessary to determine the carrier response, especially anti-PS A1 antibody level after animal immunization. Based on the primary ELISA analysis (FIGS. 9A-9B), the STn-PS A1+SAS group was chosen to investigate carrier response by using an ELISA plate coating construct of PS A1-poly-L-lysine (PS A1-PLL). As showed in FIG. 10, both anti-PS A1 IgG/IgM were detected on PS A1-PLL coated plates, and the levels of response were relatively stronger than those of anti-STn IgG/IgM. Stronger immune response of PS A1 is understandable, because the dosage of PS A1 content (18 μg) in each injection is nine times greater than that of STn moieties (2 μg), thus the dose ratio is 9/1. However, both IgG and IgM antibody ratio of anti-PS A1/anti-STn are smaller than the dose ratio, particularly for IgG. The anti-PS A1/anti-STn ratio equaled 2.3/1; the IgM ratio was 8.2/1. The IgG ratio was a very positive signal indicating that relatively balanced T cell responses between PS A1 and STn were obtained after immunization. Thus, PS A1 is very unlikely to caused epitope suppression in this case. To the contrary, the IgG ratio of KLH/STn obtained from the official Phase III report of the drug THERATOPE® (STn-KLH) is greater than 60/1.

Analysis of pIgG Subclasses

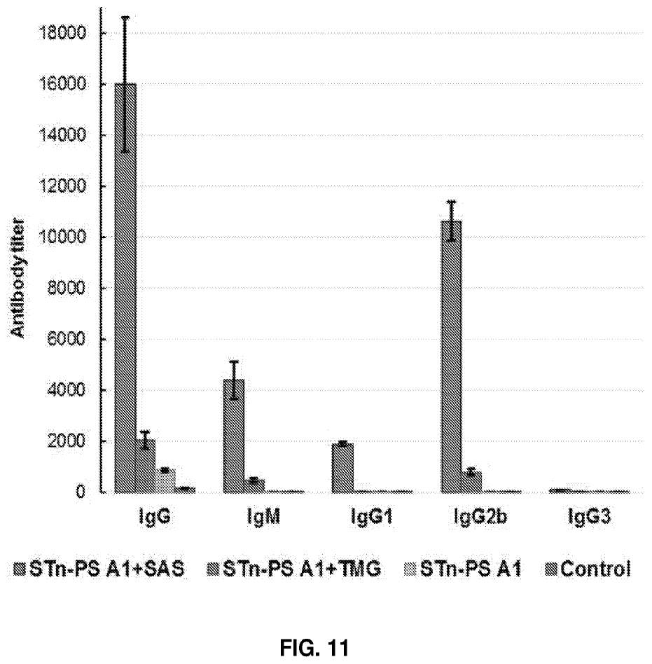

IgGs are high affinity and long-term antibodies that target many pathogens. Their subclasses exhibit slightly different immunological functions, but remain essential for complement recruitment. The subclasses of IgG induced by STn-PS A1 (16) vaccine were analyzed by a serological assay with BSM coating (FIG. 11). In the group of mice immunized with STn-PS A1 plus SAS, a substantial amount of IgG2b against BSM was observed, followed by a moderate level of IgG1, and finally a low level of IgG3 was observed when the anti STn-PS A1 sera was used. In the group of STn-PS A1 plus TMG, a moderate level of IgG2b was detected, and relatively low IgG1 and IgG3 binding events were noted. In the group of mice that were injected with only STn-PS A1 (16), negligible binding of IgG1, IgG2b, and IgG3 were detected. It is important to note that IgG2a activity was not tested due to the absence of the corresponding gene in C57BL/6 mice. These data provide further understanding of the immunological aspect of STn-PS A1 conjugates.

The high IgG2b/IgG1 ratio in both STn-PS A1 plus SAS and STn-PS A1 plus TMG groups is a strong indication that a Th1-type dominated immune response was being activated. Furthermore, the enhanced IgG2b production in the STn-PS A1 SAS murine group can be attributed to MPL as an additive adjuvant. The recognition of MPL by TLR4 on antigen presenting cells is a key event in the activation of those cells and initiation of adaptive immunity. MPL is known as a Th1-favored adjuvant and therefore can promote a Th1 response that leads to an increase in IgG2 subclass production. Since STn-PS A1 is an entirely carbohydrate construct void of proteins, peptides or lipids, the ELISA data fit into the expected immunological profile of STn-PS A1. Consequently, it is very possible that these IgG2b antibodies are specifically targeting the disaccharides moiety (Neu5Acα2-6GalNAcα) on BSM.

Antibody Binding to Cancer Cell Surfaces

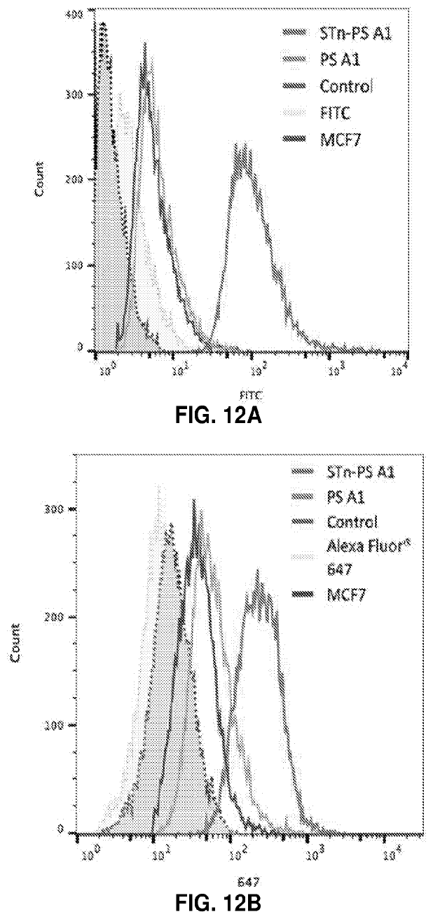

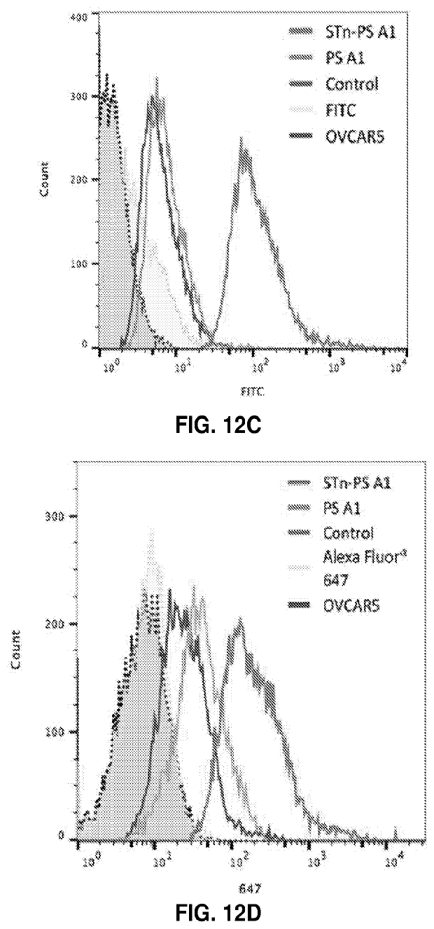

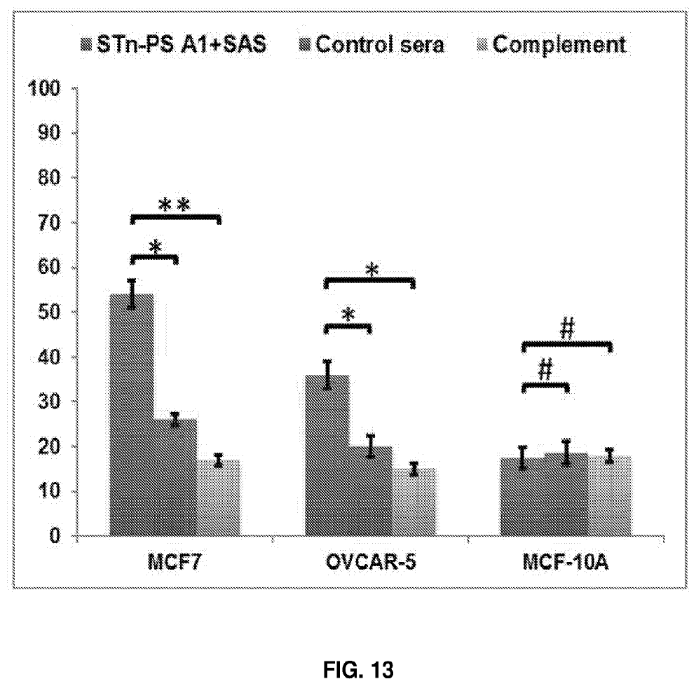

Utilization of fluorescence-activated cell sorting (FACS) is a useful method when studying the immunological potential of STn-PS A1 as a vaccine designed to target the STn antigen. Based on the serological assay and IgG subclass analyses, antisera induced by the STn-PS A1+SAS formulation was chosen for a cell surface binding test on several cancer cell lines. Cancer cells treated with anti-PS A1 serum were used as a substance control and cancer cells treated with only secondary anti-IgG (FITC) or anti-IgM (Alexa Fluor® 647) antibody were used as an isotype control. The flow cytometry results are described in FIG. 11). Human breast cancer cell line MCF-7, and ovarian cancer cell line OVCAR-5, have been proven to be STn positive cell lines. The antisera (STn-PS A1+SAS) clearly exhibited antibody reactivity against surface STn antigens using flow cytometry (FIGS. 12A-12D). The STn positive cell lines showed strong surface binding events with both IgG and IgM antibodies. The best results were observed in the IgG binding tests: the percent of positive cells for MCF-7 was 71% with enhanced mean fluorescent intensity (MFI: 155, FIG. 12A), for OVCAR-5 the percent positive was 61% (MFI: 100, FIG. 12C). IgM exhibits relatively mild binding reactivity; the percent positive was 38% for MCF-7 (MFI: 286, FIG. 12B), and 44% for OVCAR-5 (MFI: 340, FIG. 12D). In contrast, antisera obtained from the control mice only showed negligible IgM or IgG binding to the STn-positive cancer cell lines (FIGS. 12B, 12D). Anti-PS A1 sera was used as a substance control to determine any possible “epitope suppression” effects of the PS A1 “carrier” to STn antigens, and only very low/negligible binding events were detected.

Antibody-Mediated Complement-Dependent Cytotoxicity (CDC)

Based on the conclusion from the FACS assay that both IgM and IgG antibodies can be raised against the STn-PS A1+SAS formulation and are very specific in targeting STn-positive cancer cells, CDC studies were conducted. Antibody-mediated cytolysis studies are important to determine the therapeutic value of vaccine candidates. The STn-PS A1 construct was examined One of the effector killing mechanisms is through complement-dependent cytotoxicity (CDC) of certain class/subclass of antibodies, which leads to compromised tumor cell membrane integrity. The ELISA and FACS assays gave positive data regarding target validation, but did not provide an understanding of antibody function as a direct correlation to antibodies raised from STn-PS A1+SAS immunizations. First, the anti-STn-PS A1 serum contains a moderate amount of anti-STn IgM, which can be particularly effective in CDC due to the pentameric nature of IgMs. Second, there was a substantial amount of IgG2b observed in the ELISA data, which has been demonstrated to be highly potent in activating CDC compared to that of other IgG subclasses.

The results of the CDC employing MCF-7 and OVCAR-5 STn positive cell lines are summarized in FIGS. 12A-12D. The normal human mammary cells MCF-10A were used as the control cell line. The percent of lysed cells was determined using a lactate dehydrogenase (LDH) assay (Roche Applied Science) without further optimization. The substance control was settled by treating cancer cells with rabbit complement exclusively. The antisera-mediated cell lysis rate for MCF-7 was 54%, and 36% for OVCAR-5. In comparison with the CDC of antisera collected from the control PS A1 group and substance control, the antisera of STn-PS A1+SAS group was capable of inducing a significant cytotoxicity toward MCF-7 and OVCAR-5 cancer cells. There was no statically significant cytotoxicity observed on the MCF-10A cells, likely due to the absence of STn antigen.

### SUMMARY

In this Example, the preparation and immunological evaluation of an entirely carbohydrate STn-PS A1 conjugate that mimics STn-KLH is described. A highly chemo-selective and adaptive synthetic route for aminooxy-STn antigen was developed. The aminooxy sugar was conjugated to aldehyde-functionalized PS A1 through an extremely economical oxime linker. The structure of STn-PS A1 was unambiguously characterized using NMR analysis. The combination of STn-PS A1 plus Sigma Adjuvant System demonstrated a capability of inducing anti-STn antibodies in C57BL/6 mice as indicated by ELISA. FACS was also employed to study the binding events on STn expressing MCF-7 and OVCAR-5 cancer cell lines. The results from both assays further confirmed the excellent specificity and selectivity of antibodies raised against the STn-PS A1 immunogen for binding the tumor cell surface STn antigen. Moreover, data collected in an in vitro LDH tumor killing assay exhibited the therapeutic ability of anti-STn antibodies in inducing complement-dependent cytotoxicity. Combined, the results from the three assays demonstrate an approach for the development of a cancer vaccine.

### Example 2—Increasing Immunogenicity of the TF-Antigen by Targeting MGL2 Receptors Using a Bivalent Tn-TF-PS A1 Conjugate

In this Example, the importance of cancer vaccine design and development is demonstrated through an immunological investigation of monovalent Tn- and TF-PS A1 constructs, leading to a unimolecular Tn-TF-PS A1 bivalent immunogen which significantly increases immunogenicity towards the TF antigen. This additive Tn effect was also demonstrated to have enhanced IgG binding to tumor cell lines MCF-7 and OVCAR-5 in FACS analysis, and very good cytotoxicity in a CDC assay that monitored the expulsion of LDH. The enhanced immunogenicity was deciphered through studying the interaction of Tn-TF-PS A1 biotinylated probes binding to C-type lectin receptor MGL2.

PS A1, a zwitterionic capsular polysaccharide isolated from the commensal bacteria Bacteroides fragilis ATCC 25285/NCTC 9343, initiates CD4+ T cell responses. The current understanding of zwitterionic polysaccharides as immune stimulants is to rival the protein paradigm for T cell activation, bridging the innate and adaptive immune gap. Innate immunological mechanistic studies of PS A1 show interactions with toll-like receptor 2 (TLR-2)/CD282 and DC-SIGN, which are important for efficient uptake by antigen-presenting cells (APCs) or dendritic cells (DCs). While PS A1 is an immunogenic stimulant and a possible alternative to protein-based cancer vaccines, it can additionally contribute to the production of Th17 immunity. The production of Th17 cells has been shown to be important in protection against Staphylococcus aureus, Mycobacterium tuberculosis, and, in particular, cancer. The activation of Th17 immunity is bifunctional in that the production of TGF-β can influence the valuable Th17 immune response but can also influence the production of T regulatory cells (Tregs). Therefore, investigating ZPSs as “carriers” in vaccine development requires strategies to decrease the regulatory immune responses through selective modifications that allow for interactions with other innate immune receptors.

The design and development of carbohydrate-based vaccines remains important for targeting diseased states where unique sugar antigens are normally the first-line in immune surveillance. Carbohydrates, however, have long been known to be weakly immunogenic in eliciting valued T cell responses. One strategy for improving immunogenicity involves tailoring the immunogen to target specific innate immune receptors found on APCs or DCs. These immune response receptors have evolved to differentiate self-versus pathogen-associated molecular patterns (PAMPs). PAMPs are often composed of diverse glycan structures that can be broadly recognized by C-type lectin receptors (CLRs) and TLRs. CLRs are best characterized as calcium-dependent proteins expressed on myeloid cells to promote efficient antigen uptake which ultimately leads to presentation to CD4+ T cells. Fortunately, CLRs are selective for conserved carbohydrate recognition domains concomitantly leading to pathogen clearance.

A specific CLR, macrophage galactose binding lectin 2 (MGL2), serves as a valuable surface receptor for vaccine development due to selectivity towards N-acetylgalactosamine (GalNAc), the sugar component of the Thomsen nouveau (Tn) cancer antigen. The Tn antigen is an important tumor associated carbohydrate antigen (TACA) involved in the onset and progression of tumors. Since carbohydrate/TACA conjugates are known for being weakly immunogenic, targeting CLRs with a simple covalently linked sugar is useful for enhanced phagocytosis and increased immunogenicity. Therefore, attaching N-acetylgalactosamine (GalNAc) as a small molecule activator for innate and adaptive immune responses can result in an increased uptake of TACA-based vaccine constructs.

The Tn antigen has only recently been investigated for its ability to bind MGL2. A correlation between increased Tn density on MUC-6 (15 amino acid peptide fragment) and enhanced antigen uptake by APCs in comparison to a peptide fragment alone has been demonstrated. This result is believed to be the consequence of improved binding to MGL2. Furthermore, others have engineered a comprehensive polyvalent vaccine mix composed of six monomeric TACA conjugates: 1) a MUC 1-G5 peptide containing 8 conjugated Tns, 2) a Tn cluster, 3) an STn cluster, 4) a TF cluster, 5) one consisting of Globo-H, 6) one consisting of GM3, and 7) a Lewis Y immunogen. The polyvalent mixture proved to be effective in the recognition of respective TACAs, but there was no discernible immunological titer difference between the majority of monovalent (single TACA) to heptavalent (mixture of TACAs) immunizations with keyhole limpet hemocyanin (KLH) conjugates. However, there was a noted two-fold increase in IgG titer values when peptide MUC1-G5 was used in monovalent to polyvalent immunizations. The likely rationale for Tn not contributing to an adjuvant effect to other TACAs is the notion of super cross-linking CLRs, which can decrease antigen uptake and presentation and impair proper immune recognition. This effect is also observed in the natural setting with microorganisms and endogenous glycosylated MUC-1 clearance, leading to tumor evasion. The perplexity of the Tn antigen to either increase antigen uptake or increase specific antibody generation has been shown. Therefore, with the aforementioned issues in over stimulating CLRs with regards to vaccine design, incorporating Tn on a unimolecular construct with another TACA can help promote efficient antigen uptake through MGL2 and subsequently increase immunogenicity.

In addition to targeting CLRs such as MGL2, a balanced immune response is important for overall efficacy in which Th1, Th2, and Th17 can assist in antibody production. In that regard, C-type lectins have the ability to influence cytokine production and are targets for self-adjuvanting vaccine constructs. Cytokines are often associated with an induction of suppressive Treg responses initiated through interactions with TLR-2. The presence of proinflammatory markers such as IL-6, IL-4, and IFN-γ can negate side effects of Tregs. One reagent that leads to the production of IL-6 is adjuvant monophosphoryl lipid A (MPLA), which can overcome a decrease in suppressive immune responses (T regs). Although the mechanistic function of MGL2 remains elusive, a similar signalling event belongs to the family of the asialoglycoprotein receptors (ASGP-R), which initiate pro-inflammatory cytokines. Tn-PS A1 cytokine data has shown a reduced IL-10 and increased IL-4/IL-17 expression, which was found to be distinct from PS A1 alone. Furthermore, interaction with MGL2 is known to produce IL-4. It is possible that this switch in cytokine profiles can be explained as Tn interacting with MGL2, thereby providing access to an alternate processing pathway as opposed to one for PS A1 alone. Since MGL2 skews the immune response to Th2, targeting this receptor with the Tn antigen is a viable strategy to increase immunogenicity of various TACA-PS A1 constructs.

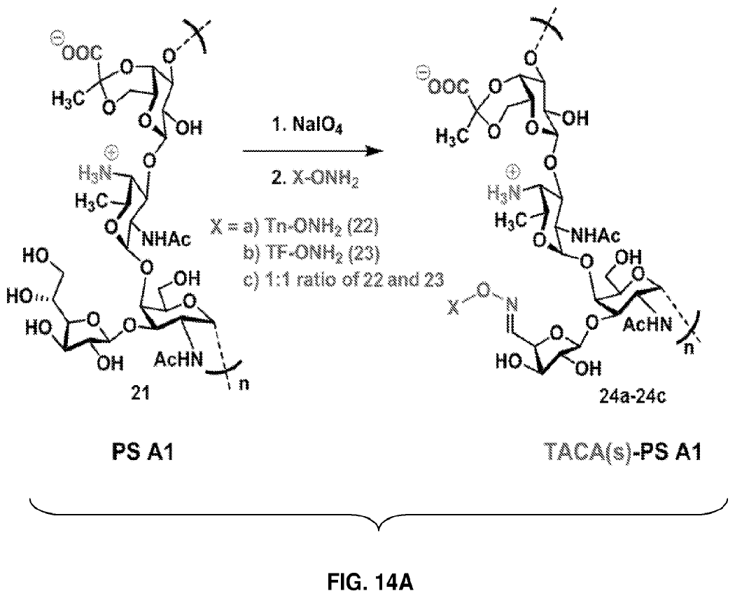

Therefore, an immunogen with dual functionality was created through: 1) an ability to increase the immune response towards TACA Thomsen-Friedenreich (TF=D-Galβ1,3-D-GalNAc) by conjugating both Tn and TF to PS A1, and 2) the use of Tn to target the MGL2 receptor. This approach has the advantage of using ZPS PS A1 (21) (FIGS. 14A-14B) which can augment multivalency effects, leading to higher degrees of interactions by Tn on the surface of APCs and thus antigen internalization. Collectively, incorporating Tn on 21 induces an adjuvant effect by involving key components of innate immune receptors such as MGL2, and subsequently activate adaptive immune responses with T and B cells. The results demonstrate this effect through increased antibody recognition of TACAs observed with anti-serum from a bivalent vaccine construct containing both Tn and TF antigens as compared to their monovalent counterparts. Without wishing to be bound by theory, it is believed that a Tn-TF-PS A1 (24c) bivalent conjugate interacts with MGL2 and increases APC uptake, thus increasing immunogenicity towards the TF antigen as opposed to monovalent TACA conjugate TF-PS A1 (24b) (FIG. 14).

Results and Discussion

The synthesis of Tn-TF-PS A1 (24c) (FIG. 14A) was achieved using sodium periodate oxidation of 21 followed by conjugation of 22 and 23 in a 1:1 molar ratio. This led to an overall loading of 29% (16.5% TF and 12.5% Tn) by mass. The loading of 24c was determined by NMR integration of the N-acetyl groups from compound 21. FIG. 14B illustrates the ¹H NMR overlays of 21 and 24a-24c to denote the chemical transformation characterized by the oxime link.

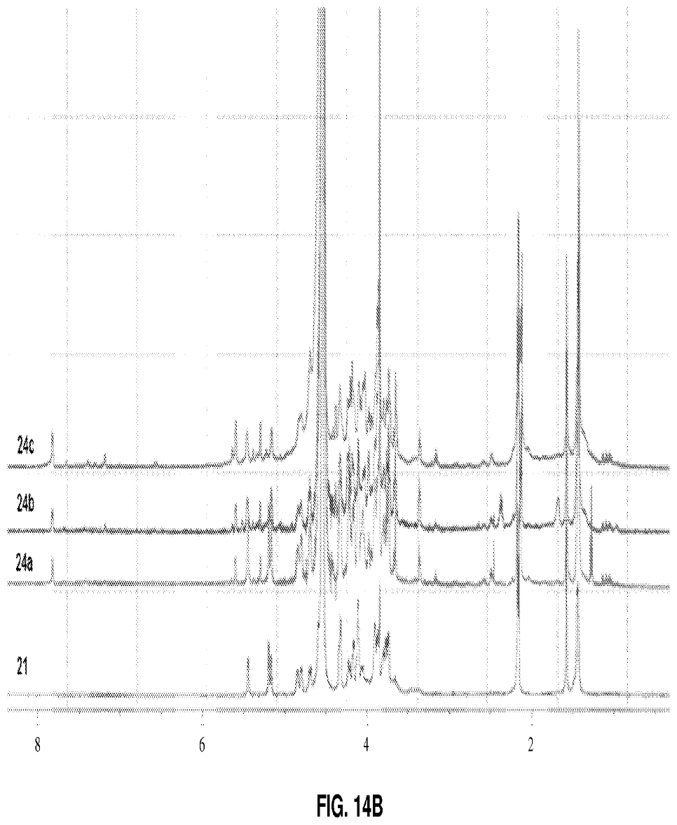

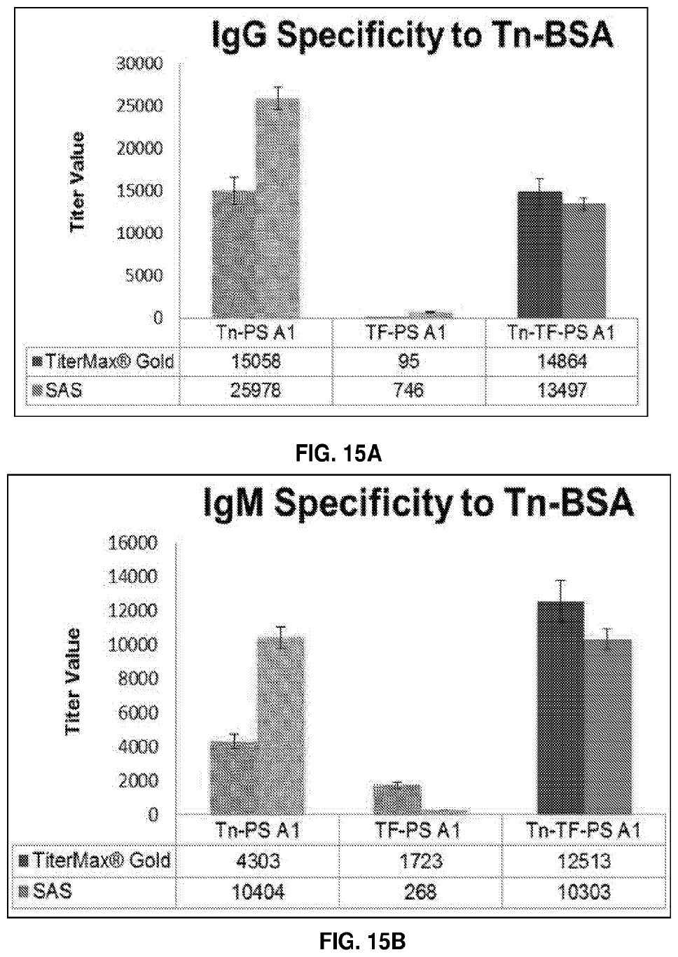

To evaluate the immunological potency of compounds 24a-24c, Jax C57BL/6 mice were vaccinated using two separate adjuvants: 1) TiterMax Gold® (TMG) and 2) Sigma adjuvant System® (SAS) to evaluate antibody binding in ELISA. Additionally, binding specificity of polyclonal antibodies towards either Tn-BSA or TF-BSA were used to parse out individual contributions of 24a-24c. First, 24a (FIGS. 15A-15B) demonstrates strong IgG/IgM specificity towards Tn-BSA when TMG is used as the adjuvant, however, the overall titer value is increased when SAS is employed. Furthermore, 24a has minimal cross-binding with TF-BSA (FIGS. 15B-15C), indicating that the immune response that is generated in mice favors the Tn antigen. However, 24b had minimal IgG binding to TF-BSA (FIG. 15C) and Tn-BSA (FIG. 15A) when both TMG and SAS were employed. Important to note is that the flexibility of the β-glycosidic linkage in the TF antigen decreases its immunogenicity. It was confirmed that there were statistically significant pIgG binding events between 24b and 21. This result verified that there was an IgG specific response generated towards TF but that the PS A1 was required for ultimate antibody stabilization.

It was initially thought that using SAS adjuvant could help boost the IgG immune response similar to the effect noticed with 24a. However, no discernible differences were observed. The immune response with 24b remained exclusively an IgM isotype with high titer values observed towards TF-BSA (FIG. 15D) and moderate cross-reactivity to Tn-BSA (FIG. 15B). An interesting caveat occurred with 24b and SAS, as it decreased IgM production compared to 24b and TMG (FIG. 15D). Since TMG and SAS have been proven to have minimal effects in producing specific IgG antibodies towards the TF antigen in 24b, a new construct had to be designed to incorporate the immune stimulating properties of 24a while a continuing focus on TF remained.

The oxime conjugation of both 22 and 23 was turned to oxidized PS A1, giving 24c (FIG. 14A). Prior to immunological evaluation, it was believed that the addition of Tn would interact with CLR MGL2 to promote a bivalent-targeted immunogen for increased antigen uptake and presentation of both Tn and TF antigens. As noted from FIGS. 15A-15D, when monovalent constructs 24a and 24b were administered to Jax C57BL/6 mice, there was minimal cross-reactivity to either immunogen. However, incorporating both Tn and TF onto PS A1 led to enhanced IgG/IgM responses when coating constructs Tn-BSA and TF-BSA were used in the ELISA. Both TMG and SAS (FIGS. 15A-15D) were used as adjuvants in separate immunization studies. While the IgG/IgM specificity towards Tn-BSA, when 24c was used as the immunizing construct, remained similar to monovalent counterpart 24a, there was a drastic change in TF-BSA IgG specificity from 24b to 24c. Moreover, 24c with SAS led to an ˜2.5 fold change from 24c when TMG was used. This indicates that in addition to Tn interacting with MGL2, MPLA (the active component in SAS) augmented the immunogenicity of 24c.

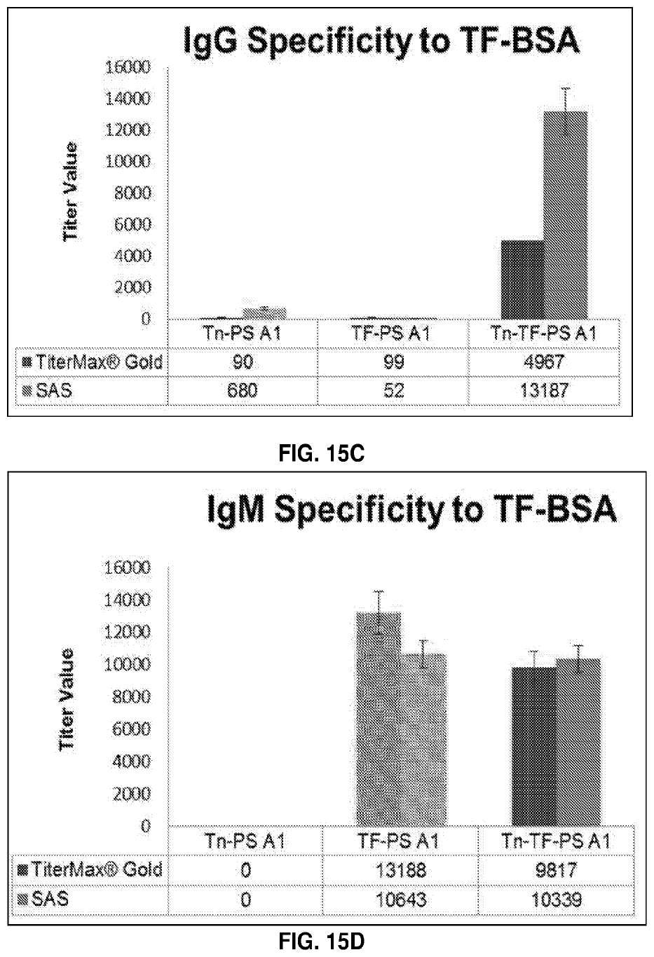

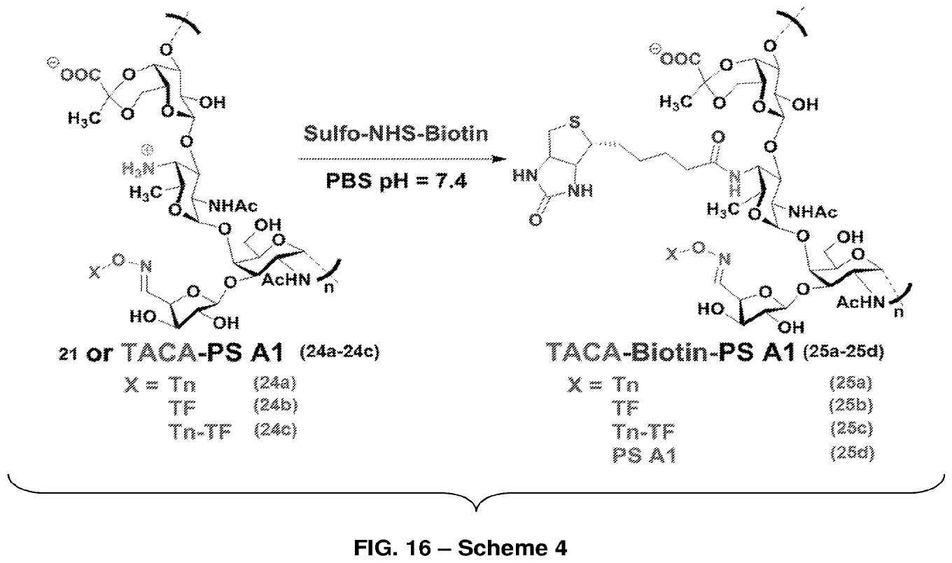

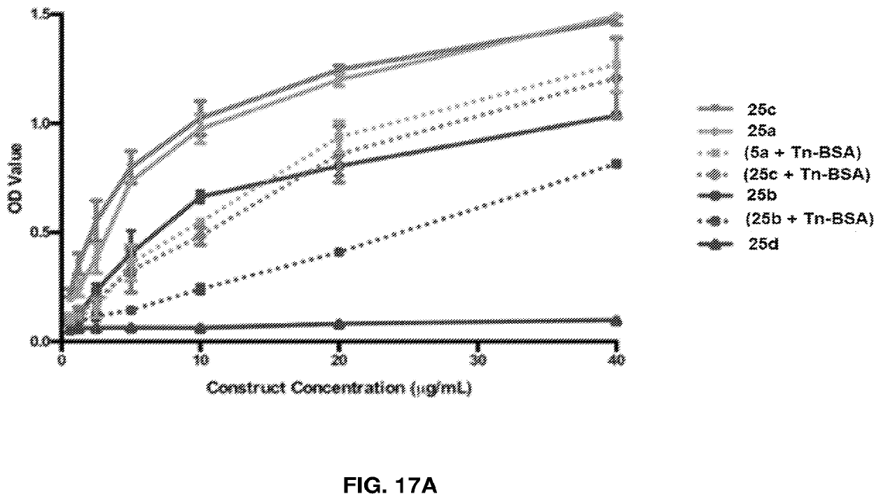

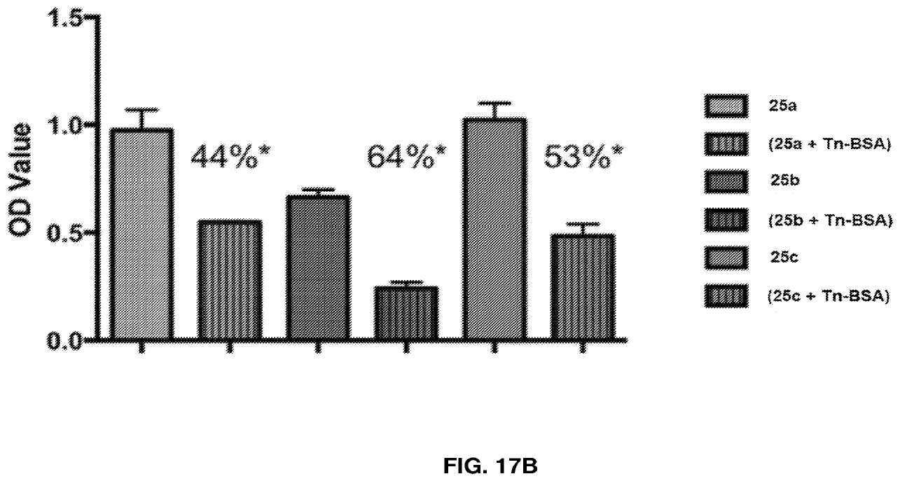

In order to validate MGL2 interaction with 24a and 24c, four biotinylated PS A1 conjugate probes (FIG. 16, Scheme 4; 25a-25d) were synthesized. The probes were constructed using oxidized 21 or 24a-24c and reacted with sulfo-NHS-biotin. Constructs 25a-25d were used in a colorimetric assay where MGL2 coated ELISA plate wells and streptavidin-alkaline phosphatase detected binding interactions. Compounds 25a-25d were evaluated for their ability to bind to MGL2 (FIG. 17A). It is important to note that 25d was used as a negative control because it is known not to interact with MGL2 and would account for a biotinylated linear probe similar to constructs 25a-25c. Only compounds 25a and 25c showed sufficient binding to MGL2 due to the presence of Tn. However, 25b showed binding that was most likely augmented by multivalent interactions with MGL2. Constructs 25a-25c (10 μg/mL) (FIG. 17B) were shown to be competitively inhibited by 10 μg/mL of Tn-BSA giving 44% inhibition for 25a, 64% for 25b, and 53% for 25c. Compound 25b was inhibited the most by Tn-BSA due to MGL2 binding preference of Tn over TF. However, 25a was favored over 25c due to the presence of TF which most likely interfered in the binding event.

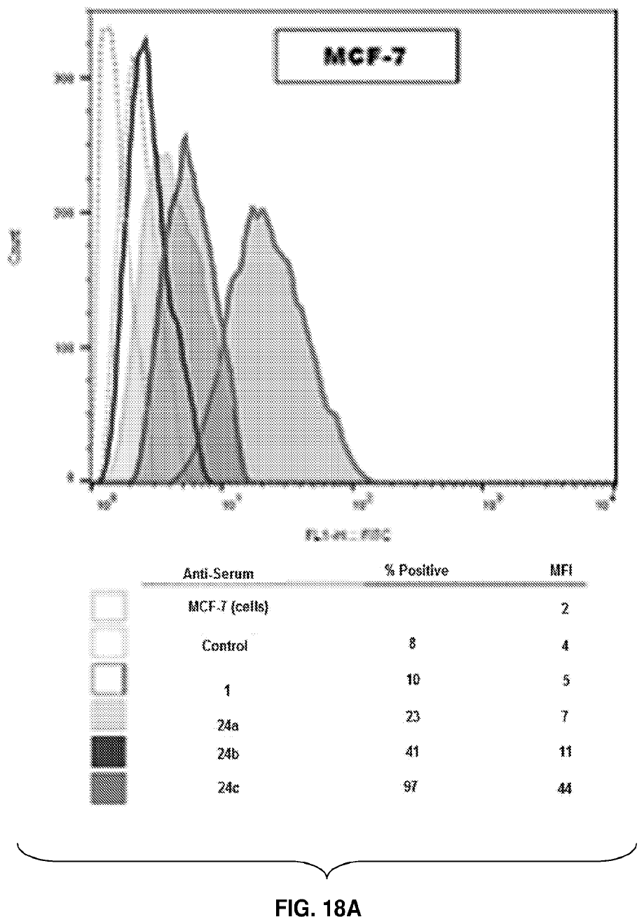

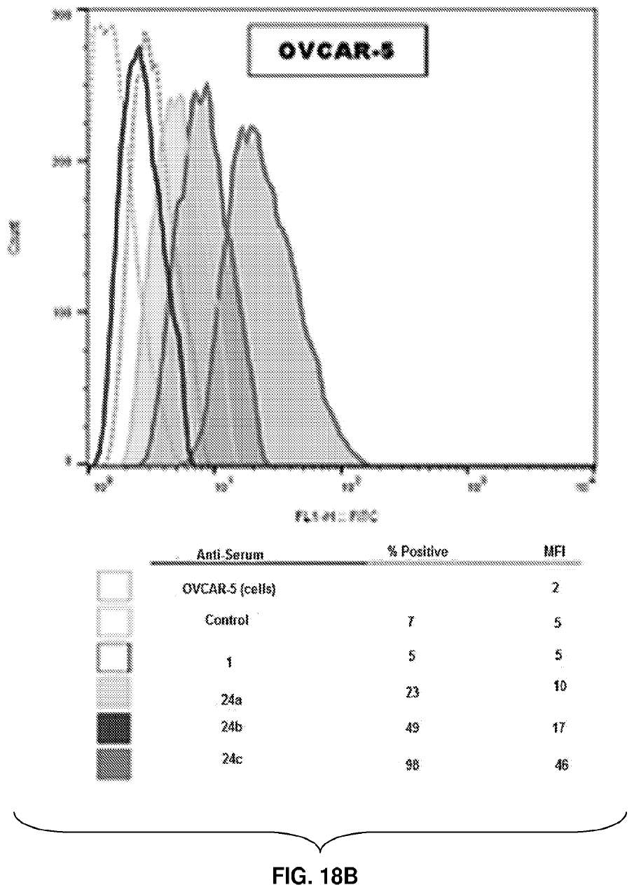

To further support the notion of increased antibody recognition through bivalent construct Tn-TF-PS A1, flow cytometry was used to determine polyclonal antibody binding to human tumor cells MCF-7 (FIG. 18A) and OVCAR-5 (FIG. 18B). Validation of the anti-serum of 24c showed a 97% gated-shift in fluorescently sorted cell populations compared to MCF-7 cells alone. For comparison, the shift in fluorescent cell populations for PBS control mice serum was 8%, 21 gave 10%, 24a gave 23%, and 24b gave 41%. Similar binding events were seen using human ovarian tumor cell line OVCAR-5 with 24c giving a 98% shift in fluorescently sorted cell population compared to the PBS control at 7%, 21 at 5%, 24a at 23%, and 24b giving 49%. The increased fluorescent cell populations of anti-24b serum to MCF-7 and OVCAR-5 comes as a surprise due to the low IgG binding to TF-BSA on ELISA (FIG. 15C).

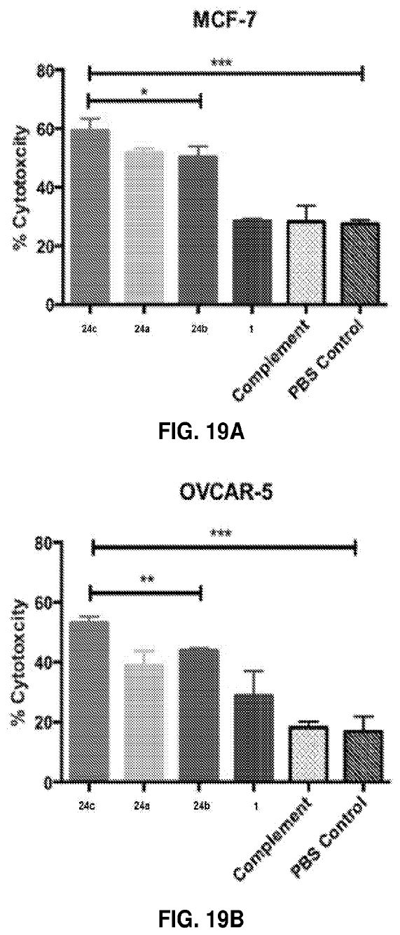

After evaluating antibody binding on flow cytometry, antibody function was assessed using complement dependent cytotoxicity (CDC) (FIGS. 19A-19B). An LDH assay was used to measure the amount of LDH released from either MCF-7 (FIG. 19A) or OVCAR-5 (FIG. 19B) by lysis of cancer cells with antibodies generated from (21 and 24a-24c) and rabbit complement. LDH is an oxidoreductase enzyme which catalyzes the conversion of lactate to pyruvate coupled with the reduction of NAD+ to NADH. Subsequently, diaphorase uses NADH to reduce iodonitrotetrazolium to formazan which can be analyzed at 490 nm. In FIG. 19A, 24c had 59% cytotoxicity towards MCF-7 with statistically significant values (P-value<0.05) in comparison with 24a 52% and 24b 50%. Additionally, 24c had 53% cytotoxicity towards OVCAR-5 which again produced statistically significant values (P-value<0.005) over 24a 39% and 24b 43%. Collectively, 24c gave a greater cytotoxicity over monovalent equivalents 24a and 24b, which is an additional advantage of an increased immune response.

The development of a bivalent Tn-TF-PS A1 construct, using a semi-synthetic approach, has led to the increased immunogenicity of the TF antigen. This increased immune response can be attributed to a targeted MGL2 strategy leading to an increased uptake of TACAs. This stands in contrast to other multivalent approaches that have been engineered in which there was no major effect on the individual TACAs alone. The success of 24c is distinct from other polyvalent immunogens (globular protein conjugates) mostly likely due to the linear and repetitive nature of 21 or 24c, leading to higher surface area contact to DCs and multivalent interactions. This demonstrates that a bivalent Tn-TF conjugate has had an enhanced immune response in increasing the binding events to TF.

Construct 24a was consistent in mounting an IgG specific immune response to the Tn antigen when TMG or SAS were used as external adjuvants. However, proving the same strategy to accommodate the TF antigen was more challenging in 24c. The results indicated that the use of adjuvants had relatively no effect on IgG titer values. When PS A1 was bivalently conjugated with both Tn and TF (24c), there was a profound difference in anti-TF IgGs compared to 24b. A comparable result was also observed in FIGS. 18-19, where the anti-serum from 24c was able to bind and contribute to the cytotoxicity of human tumor cell lines MCF-7 and OVCAR-5 greater than the monovalent equivalents. The addition of Tn signifies the importance of binding MGL2 which corresponds to higher immunological activity. To differentiate between multivalent polyclonal antibodies from 24c, either Tn- or TF-BSA ELISA coatings were screened to assess antibody specificity. To determine if MGL2 contributed to an increase in TF immune response towards 24c, four biotinylated probes 25a-25d were evaluated for binding to MGL2. Both 25a and 25c had similar binding profiles to MGL2, which signifies the addition of the Tn antigen promoted efficient uptake of the immunogen. However, the specificity to TF has been documented to have lower affinity towards MGL2, which was confirmed by examining data from FIG. 18. When 10 μg/mL Tn-BSA was used to compete with the binding of PS A1-biotin derivatives, Tn-PS A1 biotin was shown to be inhibited at 44% whereas Tn-TF-PS A1 was inhibited at 53% when an equivalent concentration of 10 μg/mL was used. The inhibition of Tn-TF-PS A1 appears to be affected by the conjugation of TF because TF has less affinity for MGL2 and therefore more susceptible to inhibition by Tn-BSA. The negative control in the experiment was PS A1 due to the fact that it has no binding value correlating to MGL2 (FIG. 18).

The rationale behind using Sigma Aldrich Adjuvant (SAS) was the incorporation of an MPLA-based adjuvant to overcome potential suppressive Treg responses. This adjuvant is distinct from TMG as a potent oil and water emulsion that allows slow release immunogen. When SAS was administered with 24c, there was increased immunogenicity to the TF antigen but also enhanced immunogenicity was observed in formulations with TMG. It is important to note the overall titers from FIGS. 17A-17B indicate the combined effects of MGL2 and SAS decrease the suppressive effects of IL-10 from the added cytokine function from MPLA based adjuvant by creating a pro-inflammatory environment and increasing IgG responses. Contrastingly, it has been previously shown that targeting TLR-2 and MGL2 separately augmented IL-10 values, an effect that was not seen with Tn-PS A1. Additionally, targeting the MGL2 receptor alone showed a decrease in production of IL-10. Therefore, the semi-synthetic modifications to PS A1 in the forms of 24a and 24c may impair TLR-2 interactions and decrease endogenous IL-10, thus promoting a pro-inflammatory immune response.

The incorporation of the Tn antigen to TF-PS A1 has had a profound influence on the respective immunological activity corresponding to an increase of the following parameters: a) IgG antibodies specific towards TF, b) binding to tumor cells, and c) complement dependant cytotoxicity. The mechanism behind this activity is increased MGL2 binding by the Tn antigen, which reveals a targeted vaccine method for enhanced antigen uptake and greater immunological activity. Since PS A1 has been noted to bind to DC-SIGN, it is a possibility that other lectins could be involved in the initiation of this immune response. This method thus has the capability of being adapted to multiple vaccines formats including peptides, proteins, nanoparticles, and lipids, to increase the therapeutic ability of carbohydrate-based vaccines.

Materials and Methods

Synthesis of Tn-TF PS A1 (24c)

A 2 mM solution of NaIO₄ was used to oxidize 1 mg of PS A1 in 0.5 mL of NaOAc buffer pH 5.2 for 90 min. KCl was used to quench excess NaIO₄. A1:1 molar ratio of Tn-ONH₂ (2) to TF-ONH₂ (3) (1.0 mg and 1.7 mg respectfully) were allowed to react with oxidized PS A1 for 24 hours followed by a long stint of dialysis using 10 kDa MWCO Snakeskin™ tubing. Percent loading was calculated from the following formula: For TF-ONH₂ (% loading=MW TF-ONH₂/MW TF-PS A1 hexasaccharide conjugate×mol fraction). The mol fraction was obtained from NMR integration of the respective two NHAc methyl protons from PS A1 and the NHAc from the TF antigen. Percent Tn-ONH₂ loading was determined by using the formula: For Tn-ONH₂ (% loading=MW Tn-ONH₂/MW Tn-PS A1 pentasaccharide conjugate×mol fraction). The mol fraction was obtained from NMR integration of the respective two NHAc methyl protons from PS A1 and the NHAc from Tn antigen.

Biotinylated PS A1/Conjugate Probes (25a-25d)

1.0 mg of either PS A1 (1), Tn-PS A1 (24a), TF-PS A1 (24b), or Tn-TF-PS A1 (24c) was reacted with 0.5 mg of sulfo-NHS-biotin (100×equivalents) (ProteoChem) in 0.5 mL of 1×PBS buffer pH 7.4 for 24 h at room temperature. The PS A1 probes were dialyzed, lyophilized, and reconstituted in 1× DPBS buffer (with CaCl₂/MgCl₂) pH 7.2 at a concentration of 1 mg/mL. Activity of the probes were evaluated in streptavidin based assays, as described below, in the MGL2 binding assay.

3-oxopropyl ethanethioate (mercaptoaldehyde) (28)

A catalytic amount of of piperidine (5.0 μL) was added to 0.5 mL acrolein (26) at 0° C. Then 0.52 mL of thioacetic acid (27) was added dropwise over a period of 30 minutes. The reaction was carried out for 12 hours and the reaction mixture was then concentrated under vacuum and purified by column chromatography using 30% EtOAc/70% DCM as the eluent to give mercaptoaldehyde (28) in 95% yield. 1H NMR (CHLOROFORM-d, 600 MHz): δ 9.67 (d, J=1.0 Hz, 1H), 3.03 (t, J=1.0 Hz, 2H), 2.73 (t, J=1.0 Hz, 2H), 2.25 ppm (d, J=1.0 Hz, 3H); ¹³C NMR (150 MHz, D₂O): δ 200.1, 195.6, 43.8, 30.7, 21.7. LRMS:ESI [M+(Na)⁺] calcd for 155.01 found 155.0.

General Procedure for TACA Linkers Tn and TF

Aminooxy Tn (22) (5.0 mg) was reacted with 2.8 mg of mercaptoaldehyde (28) for 18 h in sodium acetate buffer (pH 5.5) at room temperature and purified using Sephadex G-10 and deionized/distilled H₂O as the eluent. Fractions containing the Tn-linker were lyophilized. ¹H NMR (D₂O, 600 MHz): δ 7.51-7.54 (m, 1H), 6.89-6.92 (m, 1H), 5.37 (d, J=3.7 Hz, 1H), 5.28-5.30 (m, 1H), 4.15-4.21 (m, 1H), 3.90-3.95 (m, 1H), 3.80-3.88 (m, 2H), 3.59-3.68 (m, 2H), 2.92-3.03 (m, 2H), 2.58-2.71 (m, 1H), 2.43 (ddd, J=9.5, 6.2, 2.9 Hz, 1H), 2.24-2.29 (m, 2H), 1.91-1.97 (m, 3H), 1.80 ppm (br. s., 1H); ¹³C NMR (150 MHz, D₂O): δ 200.9, 174.5, 154.6, 104.7, 98.7, 76.8, 74.9, 72.4, 70.5, 68.5, 60.8, 47.6, 30.0, 29.3, 25.8, 25.4, 25.0, 23.2, 21.9. LRMS:ESI [M+(Na)⁺] calcd for 373.10 found 373.1.

Aminooxy TF (23) (5.0 mg) was reacted with 1.7 mg of mercaptoaldehyde (28) for 18 h in sodium acetate buffer (pH 5.5) at room temperature and purified using Sephadex G-10 and deionized/distilled H₂O as the eluent. Fractions containing the TF-linker were lyophilized. ¹H NMR (D₂O, 600 MHz): δ 7.53 (t, J=6.2 Hz, 1H), 6.91 (s, 1H), 5.37 (d, J=4.0 Hz, 1H), 5.28 (d, J=4.0 Hz, 1H), 4.40 (d, J=8.1 Hz, 1H), 4.32-4.39 (m, 1H), 4.19 (d, J=2.9 Hz, 1H), 4.16 (d, J=2.9 Hz, 1H), 3.95 (dt, J=11.2, 2.7 Hz, 1H), 3.84-3.89 (m, 1H), 3.80 (d, J=2.9 Hz, 1H), 3.59-3.69 (m, 2H), 3.49-3.58 (m, 1H), 3.42 (dt, J=9.9, 7.7 Hz, 1H), 2.92-3.05 (m, 1H), 2.57-2.75 (m, 1H), 2.44 (q, J=6.6 Hz, 1H), 2.33 (s, 1H), 2.24-2.28 (m, 1H), 1.91 (s, 1H), 1.80 ppm (s, 1H); ¹³C NMR (150 MHz, D₂O): δ 200.9, 174.5, 154.6, 104.7, 98.7, 76.8, 74.9, 72.4, 70.5, 68.5, 60.8, 47.6, 30.0, 29.3, 25.8, 25.4, 25.0, 23.2, 21.9. LRMS:ESI [M+(Na)⁺] calcd for 535.15 found 535.1.

BSA-Maleimide

1.0 mg of BSA was dissolved in 300 μL of 1×PBS buffer with 1 mM EDTA (pH 7.2) and reacted for 30 min with 100 μL of a 2 mM of 3-(maleimido)propionic acid N-hydroxysuccinimide solution in 1 mL of DMF. Excess 3-maleimidopropionate was removed by centrifugal ultrafiltration (Vivaspin® 6 MWCO 10 kDa) and washed three times with 5 mL of 1×PBS buffer containing 1 mM EDTA (pH 7.2). Conjugation was analyzed by MALDI-TOF and determined to be M/Z 71686.967. Mass loading was calculated using the following equation: (MW of BSA-maleimide−MW of BSA (664303)/(MW of maleimide linker). Based on the molecular weight, we were able to determine that there were 34 molecules of maleimide linked to BSA.

Tn-BSA (12)

2.5 mg of Tn-linker was deacetylated using Zemplen's method consisting of NaOMe in methanol followed by neutralization with DOWEX 50W×8-100 ion exchange resin. The solution was then filtered and concentrated under reduced pressure. The deacetylated Tn-linker was dissolved in 0.1 mL of 1×PBS buffer with 1 mM EDTA (pH 7.2) and added to a 1.0 mg solution of BSA-maleimide in 0.2 mL 1×PBS buffer. Conjugation was analyzed by MALDI-TOF (M/Z 78273.845). Mass loading was calculated using the following equation: (MW of Tn-BSA−MW of BSA-maleimide)/(MW of Tn-linker). From this method, we determined that there were 14 molecules of Tn-linker conjugated per BSA-maleimide.

TF-BSA

2.5 mg of TF-linker was deacetylated using Zemplen's method consisting of NaOMe in methanol followed by neutralization with DOWEX 50W×8-100 ion exchange resin. The solution was then filtered and concentrated under reduced pressure. The deacetylated TF-linker was dissolved in 0.1 mL of 1×PBS buffer with 1 mM EDTA (pH 7.2) and added to a 1.0 mg solution of BSA-maleimide in 0.2 mL 1×PBS buffer. Conjugation was analyzed by MALDI-TOF (M/Z 78273.845). Mass loading was calculated using the following equation: (MW of TF-BSA−MW of BSA-maleimide)/(MW of TF-linker). It was determined there were 14 molecules of TF-linker conjugated per BSA-maleimide. ¹H NMR (D₂O, 600 MHz): δ 7.53 (t, J=6.2 Hz, 1H), 6.91 (s, 1H), 5.37 (d, J=4.0 Hz, 1H), 5.28 (d, J=4.0 Hz, 1H), 4.40 (d, J=8.1 Hz, 1H), 4.32-4.39 (m, 1H), 4.19 (d, J=2.9 Hz, 1H), 4.16 (d, J=2.9 Hz, 1H), 3.95 (dt, J=11.2, 2.7 Hz, 1H), 3.84-3.89 (m, 1H), 3.80 (d, J=2.9 Hz, 1H), 3.59-3.69 (m, 2H), 3.49-3.58 (m, 1H), 3.42 (dt, J=9.9, 7.7 Hz, 1H), 2.92-3.05 (m, 1H), 2.57-2.75 (m, 1H), 2.44 (q, J=6.6 Hz, 1H), 2.33 (s, 1H), 2.24-2.28 (m, 1H), 1.91 (s, 1H), 1.80 ppm (s, 1H); ¹³C NMR (150 MHz, D₂O): δ 200.9, 174.5, 154.6, 104.7, 98.7, 76.8, 74.9, 72.4, 70.5, 68.5, 60.8, 47.6, 30.0, 29.3, 25.8, 25.4, 25.0, 23.2, 21.9. LRMS:ESI [M+(Na)⁺] calcd for 535.15 found 535.1.

Immunizations

Jax C57BL/6 male mice (6 weeks) were obtained from Jackson Laboratories and maintained by the Department of Laboratory Animal Resources (DLAR) at the University of Toledo. All animal protocols were performed in compliance with the relevant laws and institutional guidelines set forth by the Institutional Animal Care and Use Committee (IACUC) of the University of Toledo.

Sample sizes (n=5) were chosen based on desired amount of blood sera (1 mL/mouse). Mice were distributed randomly without bias. Criterion for inclusion of mice depended on the health status of the mouse. If mice were shown to have ascites or signs of distress the mouse was euthanized. However, no abnormalities occurred throughout the duration of the experiment.

Vaccinations with Titermax Gold Individual Tn-, TF- and Tn-TF-PS A1 constructs (20 μg) were mixed in a 1:1 ratio of 50 uL of TiterMax® Gold and injected into 7 wk old C57BL/6 mice (Jackson Laboratory) (each construct was administered individually—not mixed). Mice groups (n=5) were immunized by intraperitoneal injections (i.p.) on day 0, 14, 28, 42. Blood sera were obtained using a cardiac puncture technique on day 52.

Vaccinations with Sigma Adjuvant System

Individual Tn-, TF-, and Tn-TF-PS A1 constructs (20 μg) were mixed in a 1:1 ratio of 100 μL of Sigma Adjuvant System (Sigma-Aldrich) and injected into 7 wk old C57BL/6 mice (Jackson Laboratory) (each construct was administered individually—not mixed). Mice groups (n=5) were immunized by intraperitoneal injections (i.p.) on day 0, 21, 42, per manufacture's instructions. Blood sera were obtained using a cardiac puncture technique on day 52.

Enzyme Linked Immunosorbant Assay (ELISA)

Either Tn- or TF-BSA was coated on Immulon® Microtiter™ 4 HBX 96 well plates using 3 μg/mL in carbonate buffer (pH 9.2) and then the plates were incubated for 18 h at 4° C. Plates were washed three times with 200 μL of washing buffer (1×PBS buffer with 0.05% Tween® 20) and blocked with 200 μL of 3% BSA for 1 h. Serum from mice was initially diluted at 1:100 and then serially half-log₁₀ diluted, put into wells and incubated for 2 h at 37° C. for a final volume of 100 μL in each well. After incubation for 2 h, the plates were washed three times with 200 μL of washing buffer Alkaline phosphatase linked secondary antibodies (Anti-IgM and Anti-IgG) was used to detect primary antibodies bound to either Tn- or TF-BSA. The procedure for the use of secondary anti-IgM (Southern Biotech) were diluted (1:1000) and 100 μL were placed in wells corresponding for IgM detection and incubated for 1 h at 37° C. The procedure for the use of secondary anti-IgG antibodies (light chain, Jackson Immunoresearch) were diluted (1:5000) and 100 μL were placed into wells corresponding to light chain IgG detection and incubated for 1 h at 37° C. The plates were washed three times with 200 μL of washing buffer and p-Nitrophenyl Phosphate (PNPP) (1 mg/mL) in diethanolamine buffer (pH 9.8) was added at a 100 μL per well and incubated for 30 min and optical density was read at 405 nm using BioTek PowerWave HT Microplate Spectrophotometer. All assays were performed in triplicate. Titers were determined by regression analysis with dilutions plotted against absorbance. The titer cutoff value was set at 0.2 for titer determination, which is two times the value from control mice. Statistical analysis from ELISAs for experimental groups were compared with the controls using paired t test using GraphPad Prism 6.

MGL2 Binding Assay

Mouse recombinant MGL2 (R&D systems) 2.5 μg/mL was used to coat Immulon® Microtiter™ 4 HBX 96 well plates in 1×DPBS buffer (with CaCl₂/MgCl₂) pH 7.2 for 18 h at 4° C. The plates were then washed with 200 μL of 1×DPBS washing buffer (with CaCl₂)/MgCl₂ and 0.05% Tween 20) three times. PS A1-biotin and respective biotinylated conjugates (24a-24c) were serially diluted from 40-0.625 μg/mL and incubated for 2 h at 37° C. in 1×DPBS with CaCl₂)/MgCl₂. Plates were then washed with 200 μL of 1×DPBS buffer three times. A strepavidin-alkaline phosphatase (Sigma Aldrich) was diluted (1:1000) and 100 μL was added to each well and incubated for 1 h at 37° C. The plates were washed three times with 200 μL of 1×DPBS washing buffer and PNPP (1 mg/mL) in diethanolamine buffer (pH 9.8) was added at a 100 μL per well and incubated for 30 min and optical density was read at 405 nm. Experiments were performed in triplicate and data are illustrated as mean±s.e.m.

Percent inhibition by Tn-BSA following the same procedure noted above was then conducted, however, 10 μg/mL was co-incubated with 24a-24c before binding competition to MGL2 was attempted. Percent inhibition was calculated using equation: [(O.D of 24a-24c binding to MGL2)−(O.D. of co-incubation of 24a-24c with Tn-BSA)/(O.D. of 24a-24c binding to MGL2)]×100.

Flow Cytometry

MCF-7 and OVCAR-5 (obtained from Henry Fold Health Systems mycoplasma free) were cultured in 10% FBS RPMI 1640. 1.0×10⁶ cells of each cell line was incubated at 4° C. for 1 h in the dark with 1:50 dilution of the following separate anti-sera: 1×PBS control, 21, 24a-24c. The cells were washed three times in 250 μL of FACs buffer (2% FBS in 1×PBS, 0.001% sodium azide) by centrifuging at 1000 rpm. 100 μL Anti-IgG Alexa Fluor® 488 (1:50 dilution) was added to the cells and incubated at 4° C. in the dark for 1 h followed by three washes with 250 μL of FACS staining buffer. The cells were fixed with freshly prepared 1% paraformaldehyde and analyzed using BD Biosciences FACSCalibur at the University of Toledo Core Flow Cytometry Facility. FlowJo analysis software was used to process flow cytometry data.

Complement Dependent Cytotoxicity Assay

MCF-7 cells (1.0×10⁴) and OVCAR-5 cells (1.0×10⁴) were seeded in 96 wells plates and incubated overnight in a 5% CO₂ incubator at 37° C. The plates were washed with 2% BSA in DPBS and 100 μL of experimental anti-serum in 1:20 dilution of (1, 4a-4c, and PBS control) was incubated for 1 h. The experimental wells were washed and incubated with 10% rabbit complement (Pel-Freez) for 1 h at 37° C. The control values of the LDH assay kit (Roche) was determined from spontaneous LDH release (low control) and 1% Triton X-100 (high control) and incubated for 1 h at 37° C., simultaneously with the experimental values. 50 μL of cell supernatant was transferred to a new 96 well plate containing 50 μL of DPBS. According to manufactures protocols 100 μL of the colorimetric LDH detection reagent was added to each well and the O.D was read at 490 nm. The percentage cellular cytotoxicity was calculated by the following equation: Cell cytotoxicity %=(experimental values−low control values)/(high control values−low control values)×100.

### Example 3—Bacterial Growth and Isolation, and Purification of PS A1

B. fragilis (ATCC 25285/NCTC 9141) was purchased from Presque Isle Cultures. To begin the initial growth procedure, the bacteria were streaked on blood agar-containing BBE plates. The plates were prepared in an anaerobic glove bag in a CO₂ environment. After the cultures were initiated, the plates were transferred to an anaerobic jar with gas packs in the presence of O₂ indicator strips and placed in an incubator at 37° C.

PYG broth was used for the growth of B. fragilis. Proteose-peptone (20 g), yeast extract (5 g), NaCl (5 g), and 0.001 g of reazurin per 1 L of nanopure H₂O were autoclaved. Glucose 25% (2 mL), potassium phosphate 25% (2 mL), cysteine 5% (1 mL), 0.5% of hemin in 1N NaOH (100 μL), and 0.5% vitamin K1 in absolute ethanol (50 μL) were filtered using a 0.22 μm filter, and added to the autoclaved PYG broth. Anaerobic conditions were achieved by degassing solutions for 30 min under an atmosphere of 80% N₂, 10% CO₂, 10% H₂. A resazurin indicator was used to assure an anaerobic environment. The agar plates or liquid media were ready for inoculation as soon as the media was no longer pink in color. The agar plates were cut in sections and placed into the degassed media under an inert atmosphere. For liquid media transfer, 5 mL of culture was seeded in a degassed jar by cannulation. Every 24 hr the pH of the media was tested and adjusted to 7.2. During the first 24 h of growth, the pH would drop to 5, and 5M NaOH was used to adjust the pH in 1 mL portions until pH 7.2 was noted. A total of 20 L of bacteria fermentation was accomplished.

The growth media was centrifuged at 4,000×g for 20 min at 4° C. in 500 mL bottles. The supernatant was poured off and the cells were resuspended and washed in 500 mL of 0.15 M NaCl. Then, 500 mL of 75% phenol was stirred with the washed cells at 70° C. for 30 min. The phenol layer was separated by centrifuging at 5,000×g for 30 min at 4° C. The aqueous layer was then extracted three times with ether. After extraction the aqueous layer was concentrated under reduced pressure at 60° C., and redissovled in a minimal amount of water and subjected to dialysis for 7 days and lyophilized. The crude material was then subjected to 5.0 mg/mL of RNase (Promega) and 5.0 mg/mL DNase (Promega) in 0.1 M acetate buffer followed by 10 mg/mL of Protease (Sigma-Aldrich) to degrade any RNA, DNA, and protein. The material was then purified on two size exclusion columns and an anion exchange column. The first size exclusion column was packed with Sephacryl S-400 (GE Lifesciences) using 0.5% sodium deoxycholate, 50 mM glycine, and 10 mM EDTA (pH 9.8). Crude bacterial lysate was then loaded onto the column and 2 mL fractions were collected and analyzed by UV absorbance measuring at 220, 260, and 280, and TLC charring with anisaldehyde. Fractions containing more than 0.1 ABS at 260 and 280 nm were pooled for further purification. The fractions that showed absorbance at 220 nm were pooled and dialyzed.

The polysaccharide obtained was further purified using Sephacryl S-300 (GE Lifesciences) to remove excess buffer and further separate lipid capsular polysaccharides. UV absorbance and TLC charring again analysed fractions. Finally, the last step in the purification was the use of anion exchange chromatography. The crude PS A1 was treated with 5% acetic acid for 1 h at 100° C., loaded onto the column, and eluted with 50 mM Tris-HCl, pH 7.3 and an increasing NaCl concentration from 0 M to 2 M. Nuclear magnetic resonance (NMR) was used to determine purity, and gel electrophoresis was used to determine size and was stained with a carbohydrate staining kit.

### Example 4—Murine IgM Monoclonal Antibody Generated from Tn-PS A1 with In Vivo and In Vitro Activity

An important criterion for the consideration in generating specific anti-carbohydrate mAbs is the ability to produce antibodies that are specific for glycosides without influence from peptide/hydrocarbon linkers. To avoid the cross reactivity between carbohydrate antigens, monoclonal antibodies were generated from the zwitterionic polysaccharide Tn-PS A1 to focus the immune response specifically onto Tn.

Results

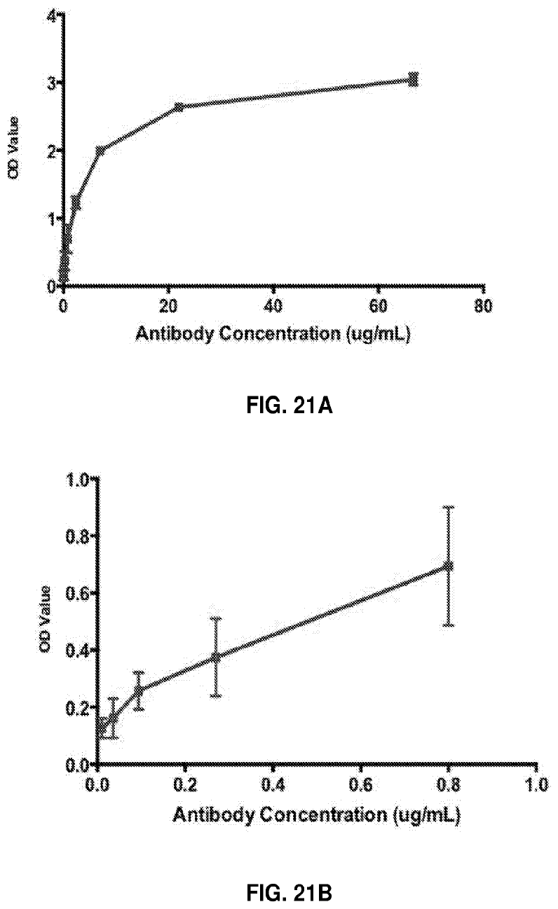

Monoclonal antibodies were generated from mice immunized with Tn-PS A1, an entirely carbohydrate immunogen. PS A1 was chosen as the immunogen because it is a zwitterionic polysaccharide that induces a T cell mediated immune response. The intended use behind this construct was to generate mAbs that are entirely based on carbohydrate binding. After immunizing mice, the spleen cells were fused with Sp2/0-Ag14 and screened the cell supernatant for the ability to bind with the Tn antigen conjugated to bovine serum albumin (BSA) in order to specifically isolate carbohydrate Tn-specific antibodies. The hybridoma cell supernatant that demonstrated the best ability to bind to Tn-BSA was chosen for scale up procedures for in vivo and in vitro evaluations. Kt-IgM-8 (IgM) demonstrated optimal binding in the titration of the antibody at 0.3 μg/mL with an optical density of (O.D.) above 0.2 (FIGS. 21A-21B). For an IgM antibody, binding at low concentrations rivals an IgG antibody but also indicates high avidity due to the pentavalent binding nature of the antibody. In order to compare the efficiency of Tn binding to a commercial antibody Tn-218 (mIgM), the same concentration of antibody was used at 30 μg/mL. In order to compare the relative binding efficiency to Kt-IgM-8. Surprisingly, from FIG. 22A, Tn-218 failed to recognize Tn-BSA, but Kt-IgM-8 demonstrated superior recognition, which indicates that the viability of binding D-GaINAc is a large improvement over what is commercially available in Tn-218. To expand upon the specificity of Kt-IgM-8, a small panel of TACA-relaxed constructs was employed that viewed various Tn-like and Tn antigens (α/β-Tn-Thr-BSA, α-Tn-BSA, α-TF-BSA, Blood Group A, and Blood Group B), which were screened using ELISA (FIG. 22A). KT-IgM-8 had no discernable binding preference between α or β containing—Thr-Tn glycosides and had decreased affinity for α-TF-BSA. Additionally, KT-IgM-8 did not bind to IPS PS A1 or BSA used to block the ELISA plates. Incredibly, Kt-IgM-8 minimally recognized Blood Group A and B below the threshold value at 30 μg/mL (O.D≤0.2) but did partially recognize them at increasing mAb concentrations (O.D≥0.2).

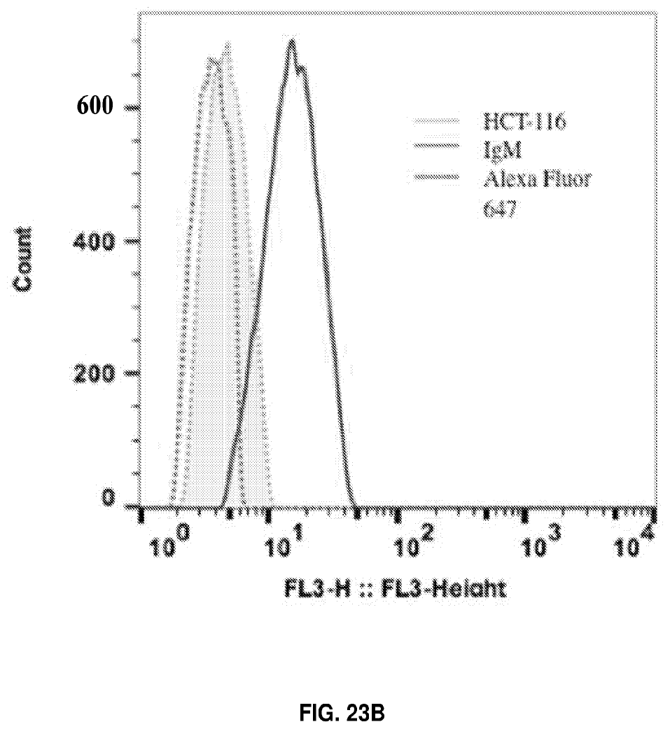

The next step in characterizing Kt-IgM-8 was to determine if the antibody could bind to whole cancer cells in flow cytometry. MCF-7 (Breast) and HCT-116 (Colon) were chosen due to both the presence of Tn and the fact that they represent two of the most common forms of cancers. Binding tumor cell lines is the first step in determining how well an immunotherapeutic will stand up against in vivo models. Anti-IgM Alexa Fluor®647 was used as the fluorescent secondary antibody to detect IgM antibody binding to the primary antibody adhered on the cancer cell lines. KT-IgM-8 shows the ability to bind to both tumor cell lines at 30 ug/mL. (FIGS. 23A-23B) and showed a shift in fluorescence of 49% in both cell lines compared to the cell lines alone.

In order to determine antibody function, a chromium-51 coupled CDC assay was used to determine the cytotoxicity of mIgM towards MCF-7 cells. In FIG. 24, Kt-IgM-8, Tn-PS A1 whole sera, (Tn-PS A1) pIgG purified from sera obtained through Tn-PS A1 murine immunizations, PS A1 sera, and a control PBS sera were used as a comparison to assess the potency of CDC activity when rabbit complement was employed. Both the Tn-PS A1 whole sera and Tn-PS A1 IgG purified polyclonal sera was used as Tn specific controls that represented cytotoxicity from whole sera and purified IgG's from the same sera. The Tn-PS A1 whole sera and the pIgG purified sera were used as controls for antibody binding to the Tn antigen BSA conjugate. Independently, they were used to represent accumulated antibody cytotoxicity from whole sera and purified pIgG's from the same sera. The purified pIgGs were important in determining how effective IgGs from Tn-PS A1 immunization could be at initiating CDC without any assistance from IgMs. Surprisingly, Kt-IgM-8 showed the greatest CDC activity at close to 30% cytotoxicity, which showed statically significant activity than Tn-PS A1 sera(P<0.005) and IgG purified Tn-PS A1 sera P<0.005) at 15% and 8%, respectively. Additionally, CDC activity was absent from anti-serums from PS A1 and PBS control mice. From an immunotherapeutic perspective, Kt-IgM-8 has the ability to initiate CDC greater than what can be seen from immunizations due to the overall concentration of antibody used. This indicates that a Tn-specific IgM antibody can provide protection to in vivo tumor models.

As a platform to examine human tumors in mice models, severe combined immunodeficient (SCID) mice are the optimal host for xenografted human tumors for immunotherapeutic evaluations, taking advantage of naturally occurring complement proteins in the absence of any functional immune system. Since the SCID mice lack B and T lymphocytes, xenografted tumors are able to be implanted and grow in the absence of an amounting immune response that would compromise tumor cell survival. Consequently, the use of MCF-7 cells represents studying breast cancer without the need for using human models. The tumor growth was measured by tumor volume (using micro-calipers) and effectiveness of the immunotherapeutic, and was assessed by the comparison of tumor volume in the control mice (PBS). FIGS. 25A-25C show four different treatments: PBS Control, KT-IgM-8 (FIG. 25A), Tn-PS A1 whole sera (FIG. 25B), and pIgGs from Tn-PS A1 immunizations (FIG. 25C). The humane endpoint of the experiment was determined when tumor volume approached 400 mm³. The control mice treated with PBS offer no protection to the tumors and determine the efficiency of each antibody treatment. The Tn-PS A1 whole sera provided the greatest protection against tumor growth at 52% difference (FIG. 25D). The Tn-PS A sera is able to use both ADCC and CDC due to the mixture of both IgM and IgG. Unfortunately, the purified pIgGs sera showed minimal protection against tumor growth. However, Kt-IgM-8 demonstrated protection against tumors at 39% difference (FIG. 25D), which significantly defines the effectiveness of the treatment. The data presented shows the effectiveness of IgM antibodies and their role in minimizing tumor growth.

### Discussion

The zwitterionic nature of PS A1 exploits a natural CD4+ immune response, which assists in a glycan-specific antibody development, which is a concept only seen in bacterial polysaccharides based mAbs. In order to confirm this, this structure was adapated to accommodate the Tn antigen. The in antigen makes the PS A1 construct more immunogenic due to the interactions with C-type lectin receptor (MGL2), which facilitates increased antigen uptake in mice. The reason for using an entirely carbohydrate immunogen (Tn-PS A1) was to focus the antibody binding on glycosides in order to generate antibodies that have no affinity towards peptides/lipids. This is a challenging endeavor because antibody binding of glycans results in low Kd values and yet the IgM antibodies compensate by having multiple binding sites, leading to higher avidity. This is a reason why IgM antibodies can be favored over IgGs when considering carbohydrates as the antigen. Targeting glycosides is essentially one of the most important features because Tn can be associated with different peptides and can ultimately effect antibody recognition. Therefore, producing a mAb that is selective for glycosides provides specificity for the Tn antigen by not having cross reactivity with peptides/lipids/proteins that are naturally occurring.

A particular concern when using an entirely carbohydrate construct is antibody cross reactivity with normally expressed carbohydrates. To examine the binding properties of the Kt-IgM-8, a small panel of in related antigens α/β-Tn-Thr-BSA, α-Tn-BSA, α-TF-BSA, Blood Group A, and Blood Group B were screened on ELISA (FIG. 22A). This panel represented different varieties of the Tn-antigen, which included the primary biological expression D-GalNAc sugar. However, α-Tn-Thr/Ser is distinctively exposed on the surface of tumor cells by a mutation in cosmc, a chaperone protein responsible for the proper folding of the glycosylation machinery. D-GalNAc is also terminally expressed on normal Blood Group A (D-GalNAc(α1-3)[Fuc(α1-2)]Gal(β1-3)), but off the carbohydrate scaffold is an adjacent L-Fuc, which may impair antibody recognition of GalNac in this confirmation. Additionally, there are similarities between Blood Group A and B, where Blood Group B has Gal substituted for D-GalNAc. A challenge associated with targeting Tn or other TACAs with antibodies is their ability to cross react with glycosides present on blood cells, which can promote harmful cytotoxicity. Furthermore, Kt-IgM-8 at 30 μg/mL does not preferentially differentiate between α/β-Tn-Thr-BSA, but α-TF-BSA does exhibit reduced binding due to the addition of the Gal to GalNAc in the disaccharide. This indicates that the antibody can recognize α/β-Tn when it is exposed on the surface, but binding is negated when Tn is masked with Gal. Kt-IgM-8 was determined to be very specific towards Tn, and thus is able to recognize Blood Group A due to the terminal expression of GalNAc. However, there was insignificant binding, which may have been impaired due to the branched structures of the Blood group antigens. Therefore, developing mAbs from Tn-PS A1 produces a very specific antibody response to the Tn antigen, and can exceed the binding produced from other mAbs made from proteins such as Tn-218 (FIG. 22B).

IgM antibodies have proven to be effective in treating carcinomas. From the data in this Example, it is shown that IgM antibodies, both monoclonal and polyclonal, may be more effective in killing tumor cells than IgG due to the potent CDC activity. In FIG. 24, Kt-IgM-8 showed a direct ability to initiate CDC compared to Tn-PS A1 whole sera and pIgG purified from Tn-PS A1 sera. In FIG. 25, SCID trice were xenografted MCF-7 tumors were treated with anti-Tn-PS A1 sera, pIgG purified from Tn-PS A1 sera, and Kt-IgM-8. The use of purified IgGs from Tn-PS A1 sera mimics IgG responses seen from vaccinations for the SCID model. The results (FIG. 25D) showed both the anti-Tn-PS A1 sera and Kt-IgM-8 were effective in reducing the size of the MCF-7 tumors with antibody treatment alone. However, the pIgGs purified from Tn-PS A1 sera showed no statistical difference in reducing the size of the tumors. These results are telling because the antibodies were naked, meaning they are not antibodies incorporated as drug conjugates and no additional cancer drugs, such as cyclophosphamide, were administered with treatment.

The IgM antibody has been overlooked for immunotherapies due to the superior nature of high affinity IgG's binding peptide/protein moieties. However, the IgG antibodies may not always be the preferred choice when it relates to glycosides, as IgM antibodies have demonstrated potent CDC responses to tumor cells. Kt-IgM-8 represents a biological tool that demonstrates in vitro complement activity and in vivo reduction of tumor growth. Additionally, only a handful of other Tn specific antibodies have been used for in vivo data, (MLS 128, GOD3-2C4, and KM3413), all of which are mIgGs. Kt-IgM-8 is likely one of the first IgM antibodies specific towards the Tn-antigen to be used in passive immunotherapies for cancer that utilizes CDC as the main source of cytotoxicity.

Experimental

Immunizations

C57BL/6 mice immunizations of Tn-PS A1, PS A1, and PBS were conducted using known methods.

Hybridoma Fusion Protocol

Mice spleens were obtained on day 60 in DMEM media. The spleenocytes were obtained by gently homogenizing the spleens. Cells were washed with serum free DMEM by centrifuging at 1000 rpm for 10 minutes and resuspending the final pellet in 30 ml of serum free DMEM. Simultaneously, Sp2/0-Ag14 (ATCC CRL-1581) were cultured and washed with serum free DMEM serum free by centrifuging at 1000 rpm for 10 minutes and resuspending in 30 ml in serum free DMEM.

2×10⁷ myeloma cells and 1×10⁸ viable spleenocytes were added in a 50 mL centrifuge tube and were washed with serum free DMEM three times. Clona Cell-HYPEG (1 ml) was added to the tube without stirring. Cells were stirred for 1 minute by gently shaking the tube. 4 mL of serum free DMEM media was added to the fusion mixture and stirred for 4 minutes. 10 mL of serum free DMEM was slowly added and incubated at 37° C. for 15 minutes. 30 mL of 10% FCS-DMEM was added and washed with 40 mL of DMEM and the supernatant was discarded. 10 mL of 20% FCS-DMEM was used to resuspend the pellet and was transferred to a T-175 flask containing 20 mL of 20-DMEM and was incubated for 24 hr in 5% CO₂. Cells were centrifuged and resuspended with 10 mL of 20-DMEM and added to 90 mL of semi-solid methyl cellulose media (ClonaCell Flex). The bottle was mixed by inverting and was aliquoted in 10 petri dishes and placed in a 5% CO₂ incubator for 10-14 days. Cell colonies were picked (5 μL) and placed in 96 well plates containing 10-DMEM in 200 μL. The cell supernatants were screened by ELISA with plates coated with Tn-BSA when sufficient antibody was produced.

IgM Purification

IgM antibodies were purified. Cell culture supernatant was dialyzed against distilled water causing a precipitation of the IgM antibody after 1 day at 4° C. The resulting precipitate was centrifuged to remove water. The precipitate was dissolved in 1×PBS buffer and was followed by ammonium sulfate precipitation by adding 17.1 g of ammonium sulfate forming a precipitate, which was concentrated and purified further with size exclusion chromatography (sephacryl S-300). Fractions were individually checked and monitored at 280 nm. The resulting fractions containing IgM antibody were pooled, sterile filtered and stored at 4° C.

ELISA

IgM Purification

Purification of IgM antibodies followed a known protocol. In short, cell culture supernatant was dialyzed using distilled water causing a precipitation of the IgM antibody after 1 day at 4° C. The resulting precipitate was centrifuged to remove water. The precipitate was dissolved in 1×PBS buffer and was followed by ammonium sulfate precipitation by adding 17.1 g of ammonium sulfate, which was concentrated and purified further with size exclusion chromatography (Sephacryl™ S-300). Fractions were individually checked and monitored at 280 nm. The resulting fractions containing IgM antibody were pooled, sterile filtered and stored at 4° C.

Complement Dependent Cytotoxicity

MCF-7 cells (2×10⁴) were adhered to a 96-well plate overnight. Cr was exposed to the cells for 4 hrs and washed with cell media. 100 μL of KT-IgM-8, anti-Tn-PS A1 whole sera, purified anti-Tn-PS A1 IgG, anti-PS A1, and anti-PBS sera was added to each well and was done in triplicate. The antibodies were incubated for 1 h at 37° C. in 5% CO₂ incubator, and the cells were washed and 10% complement was added to each well. Cr release was measured after 18 h by liquid scintillation to quantify Cr release and % cytotoxicity was calculated by using the following formula: (experimental−spontaneous)/(max−spontaneous)×100. Spontaneous wells only received media.

Flow Cytometry

mAb was diluted to 30 ug/mL and incubated with the cell lines (MCF-7 and HCT-116, 2.0×10⁶) for 30 min on ice and washed three times. Cells were labeled with either Alexa Fluor® 647 and acquired using BD FACSCalibur™ and analyzed using FlowJo software.

SCID Mice Tumor Implantation and Adoptive Transfer of Immunotherapeutic

SHO™ mice (Crl:SHO-PrKdc^{Scid}Hr^{hr}) (Charles River), female/4 weeks old, were surgically implanted with 17β-estradiol 60 day release pellet (0.72 mg/pellet) (Innovative Research of America) behind the shoulders. Two days later, 5×10⁵ MCF-7 (tumors cells) with mixed with Geltrex® Matrix (1:1) at 4° C. and subcutaneously injected into mice on their flanks (2×per mouse). The mice tumors were measured three times a week using micro-calipers and the equation (Tumor Volume mm³=(Length×width²)/2). Four days after tumor implantation, Tn-PS A1 whole sera, purified pIgGs from Tn-PS A1 sera, PBS, and Kt-IgM-8 were I.P. injected once every week until the humane endpoint was reached. Data was analyzed using GraphPad Prism, and Student t-tests were performed for determining statistical significance.

The SCID model allows for xenographed human tumors into mice, and the use of the MCF-7 breast carcinoma cell line represents treatment potential. FIGS. 23A-23B show the complement protection from IgM antibodies, which is often overlooked and originates from the innate immune responses. Targeting glycosylations by mAbs is preferentially done by the Fab (fragment antigen-binding) portion. Often, the recognition by the mAb is hindered by the use of protein/peptides, creating antibodies that have greater preference for the peptide over the glycosylation. However, to produce a true mAb towards a glycosylation, Tn-PS A1 was used.

These results are excellent for breast oncology because the MCF-7 cells had reduction in size. One reason why the Tn-PS A1 and -sera had a profound reduction in tumors compared to Kt-IgM-8 was the order of magnitude difference in protein concentration. However, the Tn-PS A1 purified anti-IgG did not induce sufficient ADCC, as demonstrated by the growth of the tumors compared to PBS

Kt-IgM-8 Heavy and Light Chain Sequencing

Light Chain:
(SEQ ID NO: 1)
CAAATTGTTCTCACCCAGTCTCCAGCAATCATGTCTGCATCTCCAGGGGA
GAAGGTCACCATGACCTGCAGTGCCAGCTCAAGTGTAAGTTACATGCACT
GGTACCAGCAGAAGTCAGGCACCTCCCCCAAAAGATGGATTTATGACACA
TCCAAACTGGCTTCTGGAGTCCCTGCTCGCTTCAGTGGCAGTGGGTCTGG
GACCTCTTACTCTCTCACAATCAGCAGCATGGAGGCTGAAGATGCTGCCA
CTTATTACTGCCAGCAGTGGAGTAGTAACCCGCTCACGTTCGGTGCTGGG
ACCAAGCTGGAGCTGAAA.
Heavy Chain:
(SEQ ID NO: 2)
CAGATCCAGTTGGTACAGTCTGGACCTGAGCTGAAGAAGCCTGGAGAGAC
AGTCAAGATCTCCTGCAAGGCTTCTGGGTATACCTTCACAACCTATGGAA
TGAGCTGGGTGAAACAGGCTCCAGGAAAGGGTTTAAAGTGGATGGGCTGG
ATAAACACCTACTCTGGAGTGCCAACATATGCTGATGACTTCAAGGGACG
GTTTGCCTTCTCTTTGGAAACCTCTGCCAGCACTGCCTATTTGCAGATCA
ACAACCTCAAAAATGAGGACACGGCTACATATTTCTGTGCAAGACATTAC
TACGGAGGGGCTTACTGGGGCCAAGGGACTCTGGTCACTGTCTCTGCA.

Kt-IgM-8 Glycopeptide Array

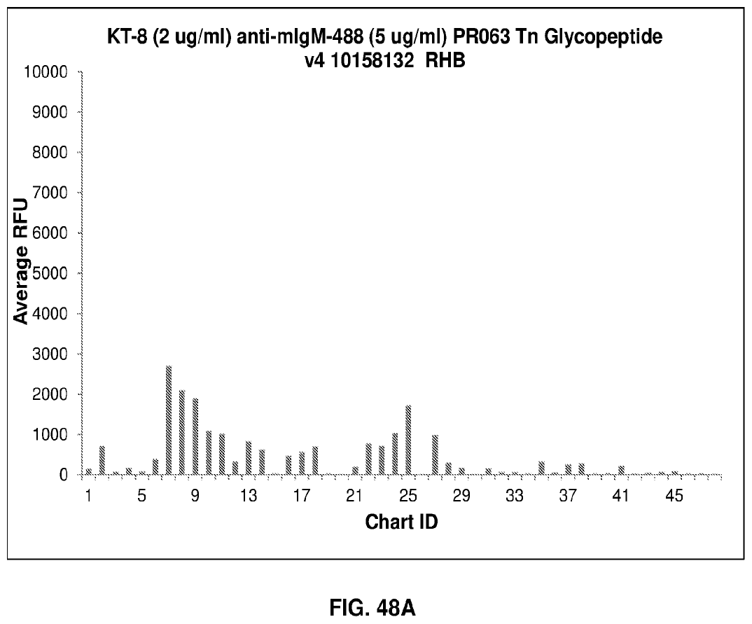

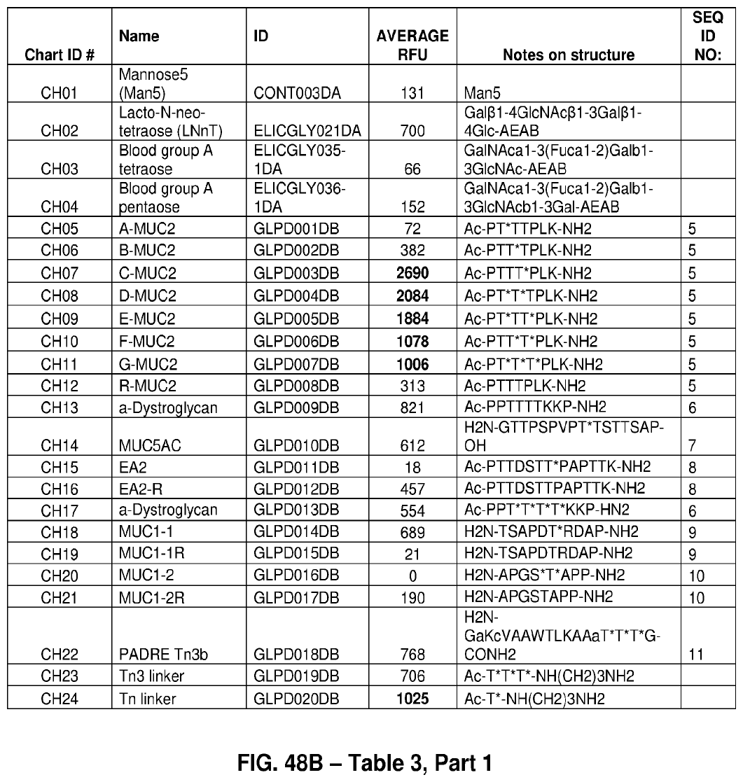

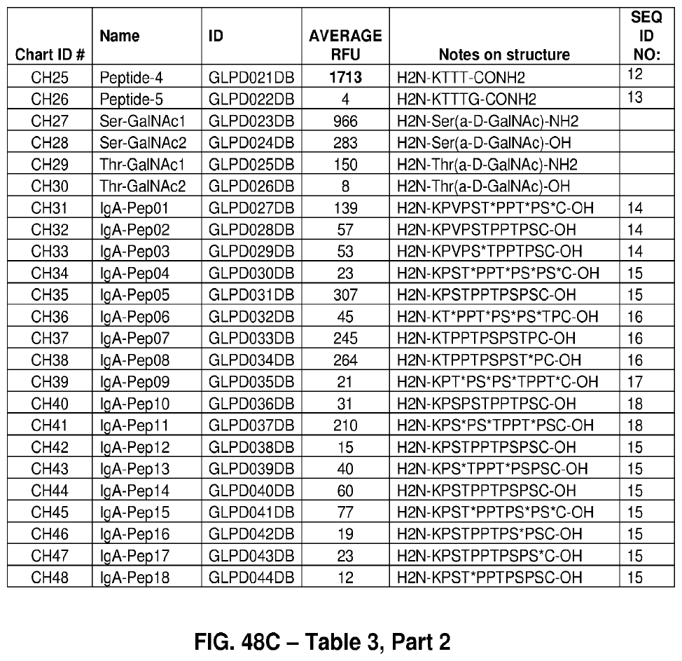

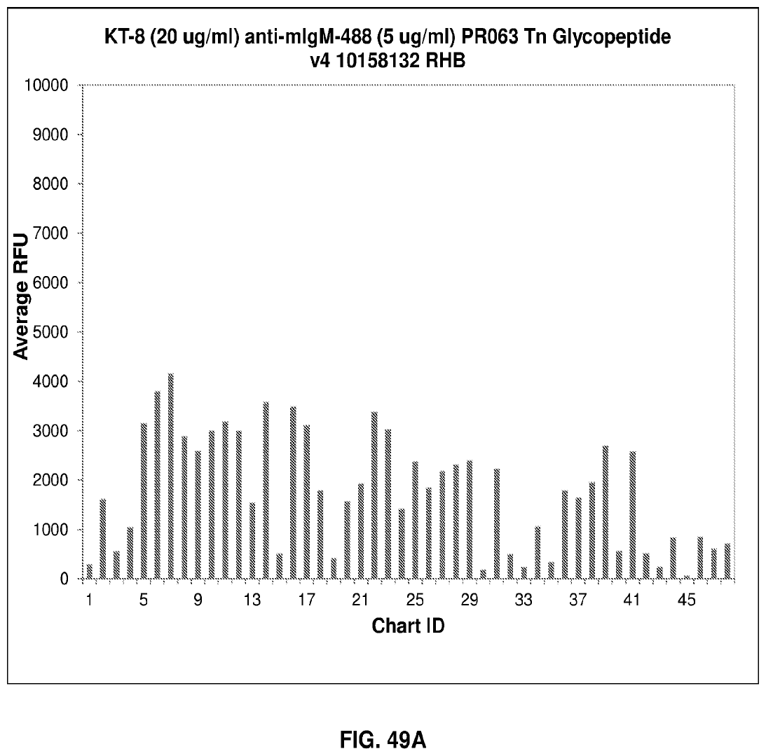

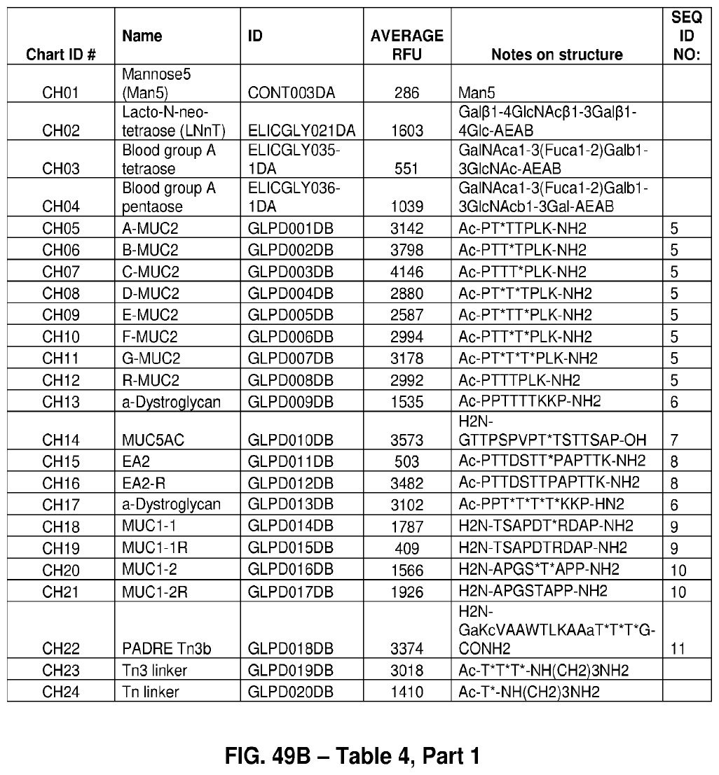

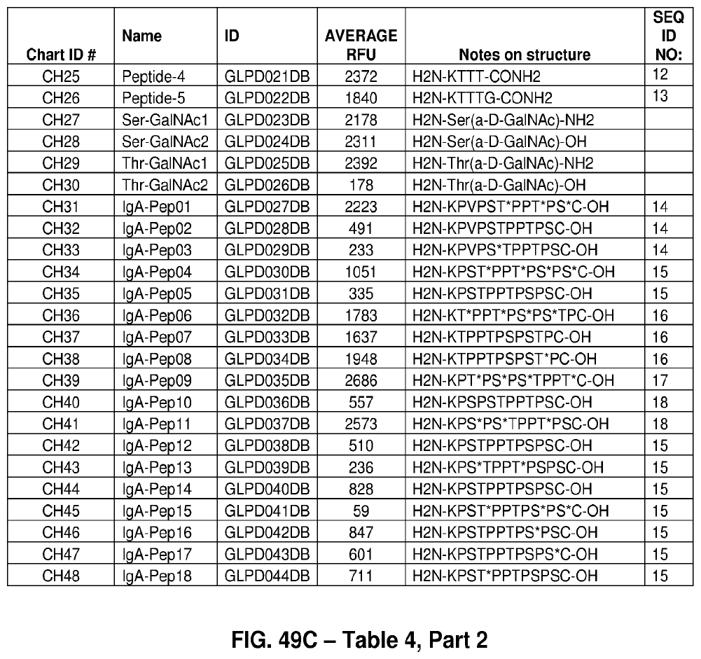

Antigen arrays provide a high-throughput platform for analyzing binding to numerous antigens. The glycan binding specificity of Kt-IgM-8 was analyzed with a glycopeptide array, at antibody amounts of 2 μg and 20 μg. The results are depicted graphically in FIGS. 48A, 49A, which show binding through average relative fluorescence units (RFU), and summarized in Tables 3-4 (FIGS. 48B-48C, 49B-49C). FIG. 48A shows the graph of RFU for the different glycopeptides at an antibody amount of 2 μg, and FIGS. 48B-48C show Table 3, displaying a summary of the glycopeptide array data depicted in FIG. 48A by chart ID number and structure. FIG. 49A shows the graph of RFU for the different glycopeptides at an antibody amount of 20 μg, and FIGS. 49B-49C show Table 4, displaying a summary of the glycopeptide array data depicted in FIG. 49A by chart ID number and structure.

### Example 5—TF-PS B

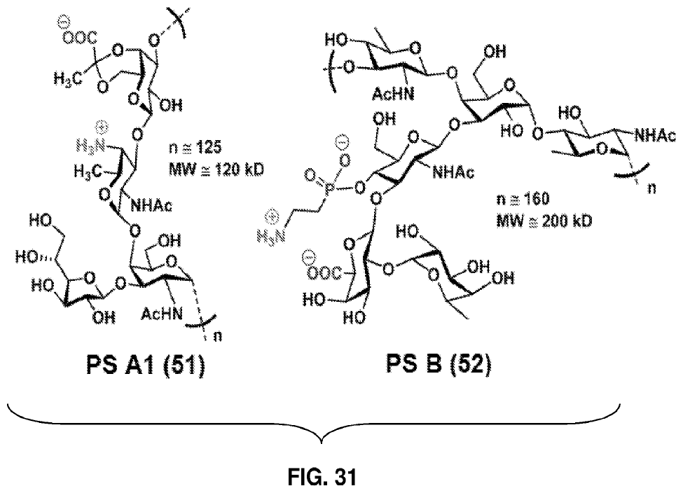

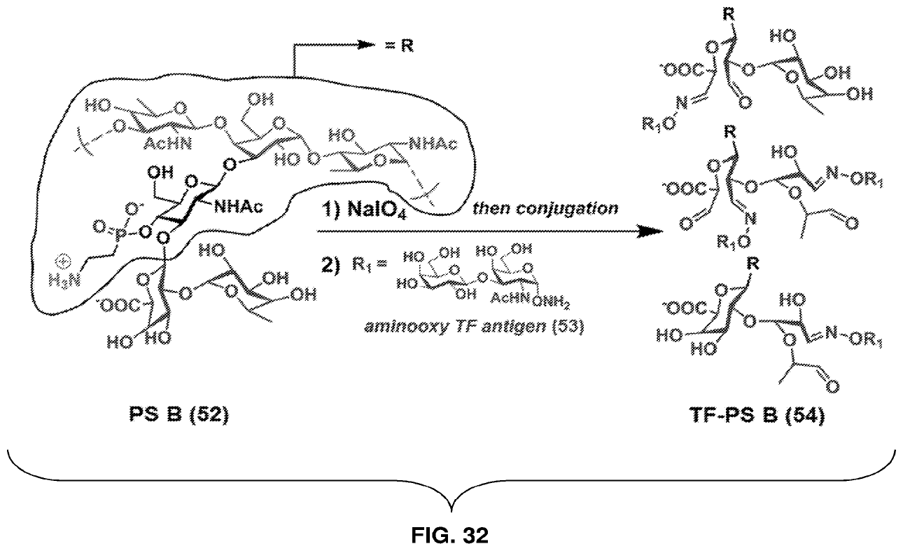

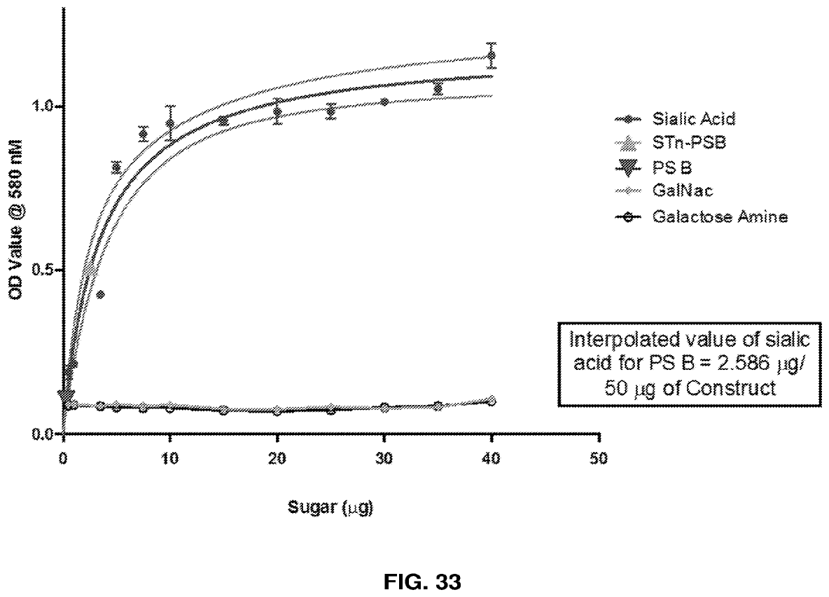

PS B (52) was oxidized using the Malaprade reaction with 10 mM sodium periodate in NaOAc buffer (pH 5) to reveal aldehydes that specifically reat with aminooxy-TF antigen (53) for conjugation to produce TF-PS B (54) (FIG. 31). An oxime bond was chosen for purposes that include stability and efficiency. Once the conjugation was complete, as noted by ¹H NMR following the formation of oxime characterization, the percent loading of the TF antigen on PS B was determined. Determining percent loading without the capability of mass spectroscopic techniques (polysaccharides do not ionize well) can be challenging, however, to overcome this limitation two indirect methods known for quantitative analysis pertaining to percent loading were employed. First, a periodate-rescorinol sialic acid assay using STn-PS B was used. Second, an Alexa Fluor® 488-hydrazide fluorophore conjugation protocol was used. In the first method, the periodate-rescorinol strategy was preferred over a phenol-sulfuric acid method as the latter is non-specific towards carbohydrates and PS B posed some interference in the development of a calibration curve. Sialic acid based conjugates are optimal in this scenario and are preferred over the TF-antigen (53) due to the vicinal diols of sialic acid requiring low sodium periodate concentrations for oxidation (1 mM), where (52 and 53) require higher concentrations (10 mM) of sodium periodate, leading to undesired fluorophore generation. In short, a standard curve of sialic acid was used to interpolate the concentration of sialic acid on STn-ONH₂ conjugated to PS B from the in situ fluorophore generated using the periodate-rescorinol method between aldehydes and rescorinol which gave ˜10% loading (FIG. 33). PS B, GalNAc, and Galactose amine were used as controls to demonstrate the selectivity of this assay towards sialic acid. The Alexa Fluor® 488-hydrazide labeling method gave ˜6% by conjugating the reactive fluorophore via a hydrazone linkage directly to oxidized PS B to be quantified. The difference in loading levels can be attributed to steric hindrance affiliated with the bulky Alexa Fluor® 488-hydrazide binding in close proximity to available oxidized PS B carbonyl aldehydes and electrostatic repulsion from the nature of Alex Fluor® 488 itself.

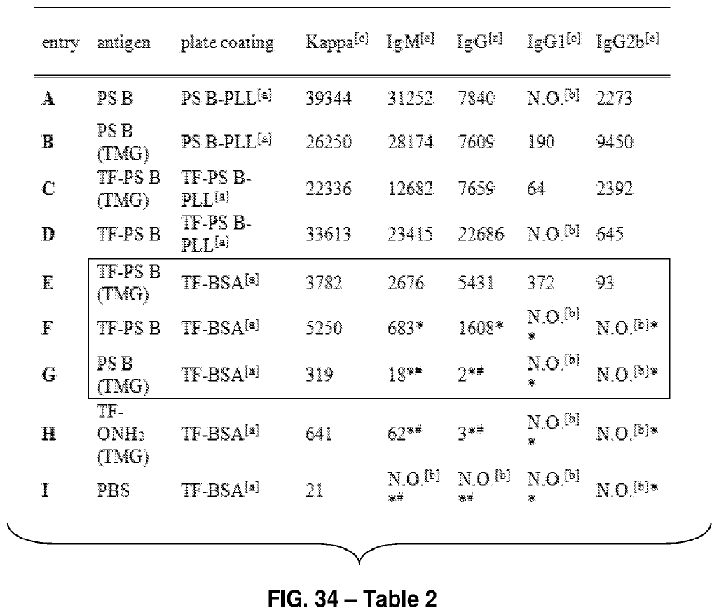

In order to determine the effectiveness of the TF-PS B construct (54), Jax C57BL/6J mice were immunized, blood sera were collected, and anti-TF immune responses were examined Three different immunogens were administered to the mice: 1) PS B (52), 2) TF-PS B (54), and 3) TF-BSA (55) (FIG. 37) with and without TiterMax® Gold adjuvant to determine, amongst a host of assays, antibody binding and specificity towards the TF antigen. To identify the specificity and selectively of an antibody immune response, the ELISA (FIG. 34) represents the first way to detect and quantify an antibody response. Data obtained from these assays can provide in vitro insights into the specificity and selectivity to the TF hapten generated from vaccine immunizations. The general procedure for ELISA begins coating a 96-well plate with TACA-protein conjugates to measure antibody binding to a hapten (TF without PS B). The primary anti-serum from immunizations was serially diluted and incubated on the well plate containing TACA-BSA conjugates. The plate was washed to remove primary antibodies and enzyme-linked secondary antibodies were incubated with the purpose to detect bound primary antibodies. The plates were again washed to remove unbound secondary antibodies and a substrate such as 4-nitro phenylphosphate was added to the enzyme (alkaline phosphatase) linked secondary antibody to cleave the phosphate to produce p-nitrophenol chromophore which can be monitored at 405 nm.

The ELISA results, as noted in FIG. 34, entry A, indicate mice immunized with PS B alone produced titers with the respective isotypes of IgM, IgG1, and IgG2b, which is indicative of a Th1-type immune response. This convention was determined using an ELISA plate coating construct of PS B-poly-L-lys (PSB-PLL). Interestingly, when PS B was administered with TiterMax® Gold (TMG) adjuvant, there was an observed decreased titer of Kappa antibodies but an increase in IgG1 titers (FIG. 34, entry B). This is attributed to the adjuvant emulsion of TiterMax® Gold permitting slow release of PS B while promoting antigen presenting cells to the site of delivery. TF-PS B (FIG. 34, entries C-F) constructs produced similar antibody isotype profiles in comparison to PS B with the caveat of specific antibodies recognizing the TF antigen. Data for entries E-I in FIG. 34 were obtained using a TF-BSA (55) ELISA coating construct to screen for selectivity.

To validate that antibodies generated from TF-PS B construct, with or without TMG, could recognize the TF antigen alone, sera from PS B TMG immunizations (FIG. 34, entry G) was screened using a TF-BSA (FIG. 37) coating construct. Significant IgG/IgM binding difference (P<0.005) to TF-BSA were observed with entries E and F compared to control entries H and I. Therefore, the antibodies generated from TF-PS B construct (FIG. 34, entries E and F) were noted as being selective and specific in regards to the TF antigen (53).

Since polyclonal antibodies from anti-TF-PS B (TMG) immunized mice (FIG. 34, entry E) recognize the TF-antigen, it was important to compare the efficacy of the anti-TF antibodies generated from a common protein construct in order to parse out the PS B binding contribution of the TF-PS B immunogen. A TF-BSA conjugate (FIG. 37) was prepared, where TF (53) was reacted with mercaptoaldehyde (56) to yield the TF linker (57). Zemplen conditions were used to deacetylate the thioacetate to compound 58, which was used to react with BSA-maleimide to afford semi-synthetic TF-BSA. There was a loading of 34 molecules of TF per unit of BSA determined by MALDI-TOF and was consequently immunized in Jax C57BL/6 mice.

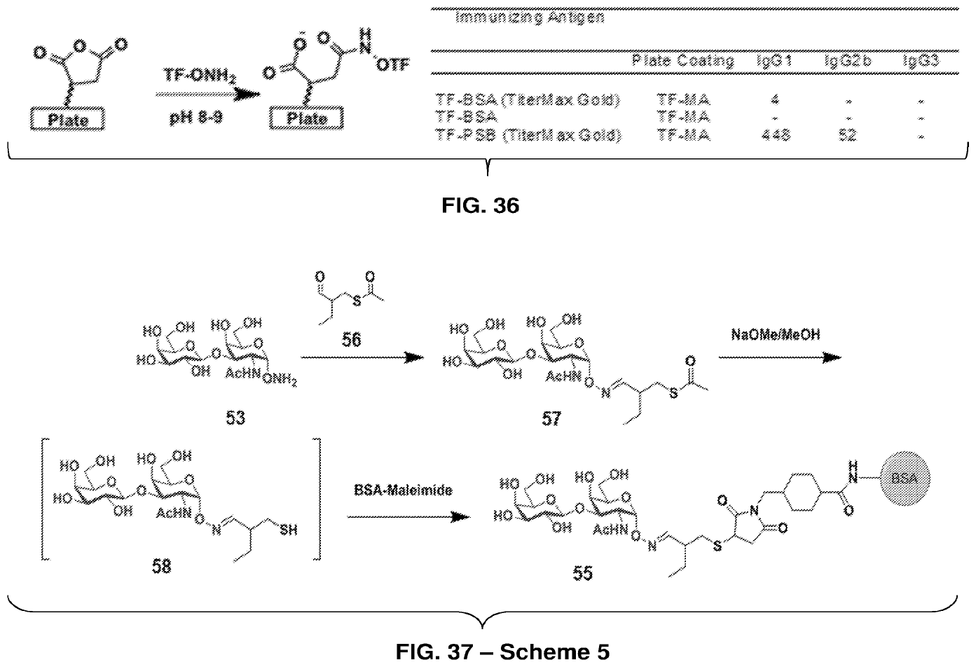

In this case, when the anti-IgG isotypes from TF-BSA immunization were specifically examined, an observable larger titer response was generated towards TF-PS B (with TiterMax® Gold) (FIG. 36) when a TF-maleimide coating was used (Pierce™ Maleic Anhydride Activated Plates). ELISA with TF-maleic anhydride (TF-MA) coated plates were used to determine the titer of TF-PS B (54) and TF-BSA (55). TF-maleic anhydride plates were used because TF-ONH₂ (53) could be conjugated to the maleic anhydride (MA) coated plates without the need for protein conjugates or linkers, therefore allowing for true recognition of the TF-antigen. Another method for screening TF was used (TF-KLH), and it was constructed using similar conditions to TF-BSA but it was concluded that the maleimide linker augmented the binding data and subsequently the titers. Therefore, maleimide free ELISA coated plates were required to determine the specificity and selectivity of TF-BSA immunizations. From FIG. 36, the TF-MA plates were used as a common platform to compare the titer data from TF-BSA and TF-PS B. The data from TF-PS B was similar to what was seen in (FIG. 34, entry E) but anti-TF-BSA antibodies had minimal IgG recognition to TF. A possible explanation for this observation is TF-PS B may be able to act as a bridge between the innate and adaptive immune responses, producing specific anti-TF antibodies. TF-BSA contains a (4-maleimidmethyl)cyclohexane-1-carboxylate linker, which is known to elicit strong immune responses against the linker and suppressing the immune response against the carbohydrate based TF antigen.

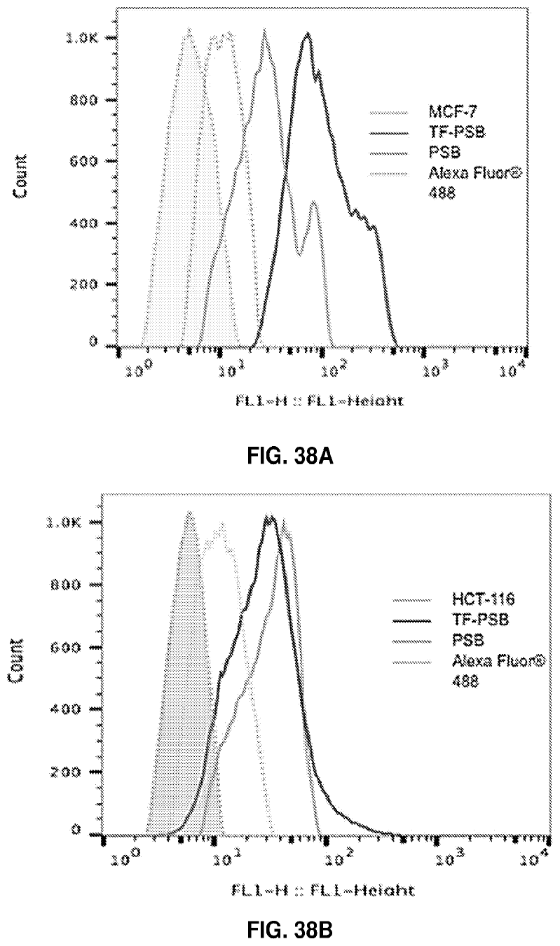

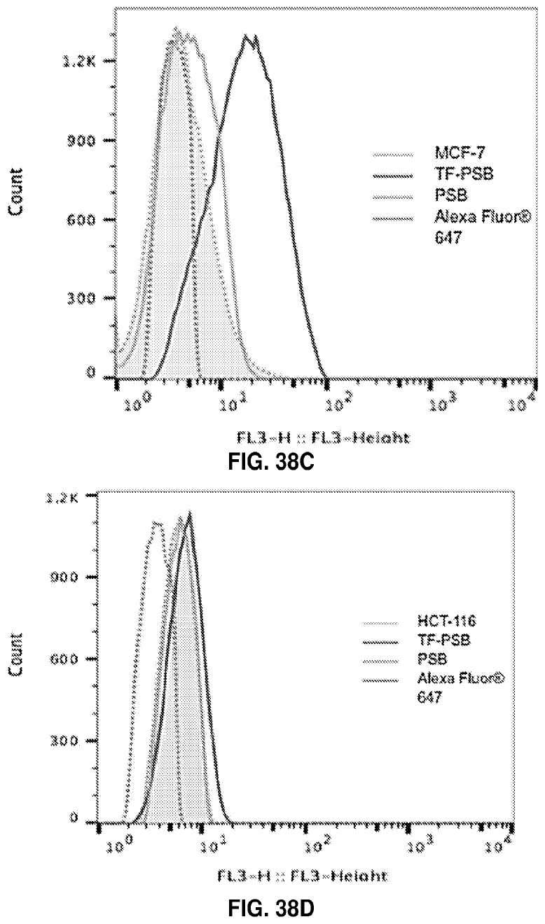

Based on the ELISA results, which concluded that a TF-PS B construct could elicit selective TF binding polyclonal antibodies generated in mice, the binding preference of those antibodies towards a human MCF-7 (breast) tumor cell line was examined. It is known that MCF-7 cells express the TF antigen. To achieve this aim, a fluorescent binding technique was employed, and flow cytometry was used to determine binding efficiency. The principles of FACS (Fluorescent Activated Cell Sorting) are similar to the ELISA technique but primary antibodies and secondary fluorescently labeled cells can be sorted based on fluorescent intensity. The TF-PS B (with TMG) anti-serum (blue line) produced higher fluorescent IgM/IgG binding events to MCF-7 (FIGS. 38A and 38C) than did PS B (with TiterMax® Gold) serum alone (orange line). However, PS B had more fluorescent IgG events than TF-PS B on the HCT116 (colon) cells. Both TF-PS B and PS B anti-IgM (FIG. 38D) responses did not sufficiently recognize HCT-116. A rational explanation for the larger IgG recognition of anti-PS B over anti-TF-PS B (FIG. Q7B) is believed to be because HCT-116 expresses lower quantities of the TF-antigen (CD176) compared to MCF-7, and is a possible reason for the disparity in antibody recognition for different carcinomas.

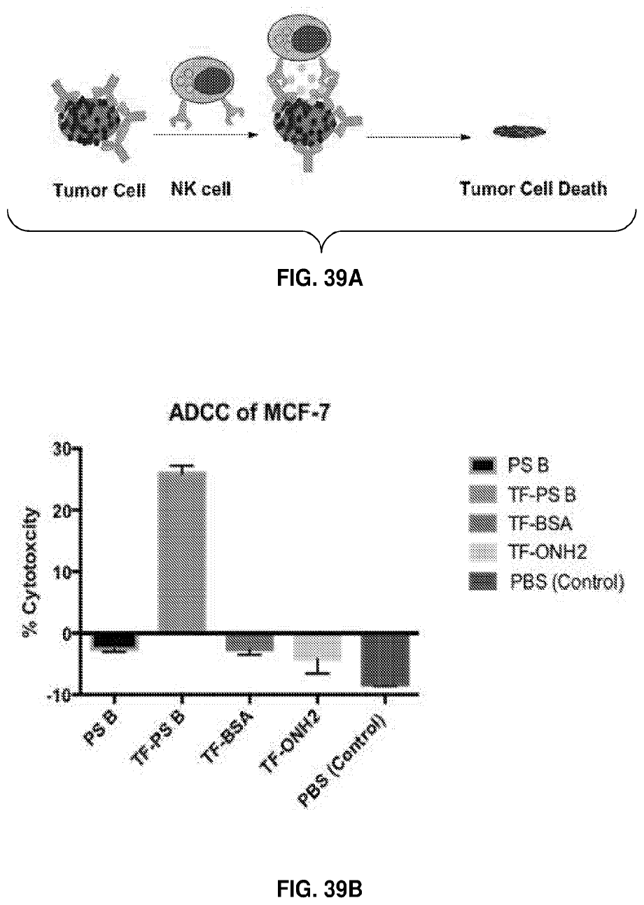

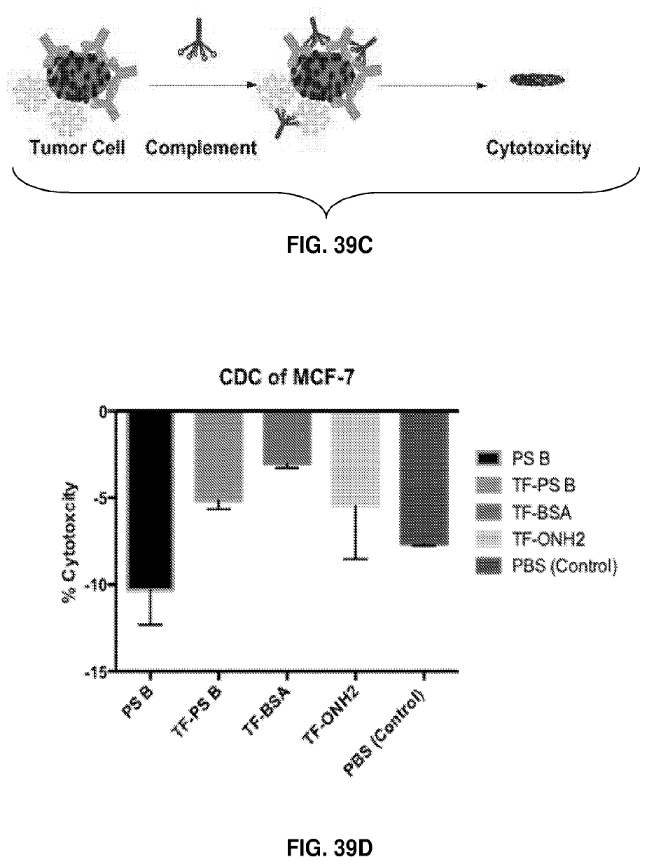

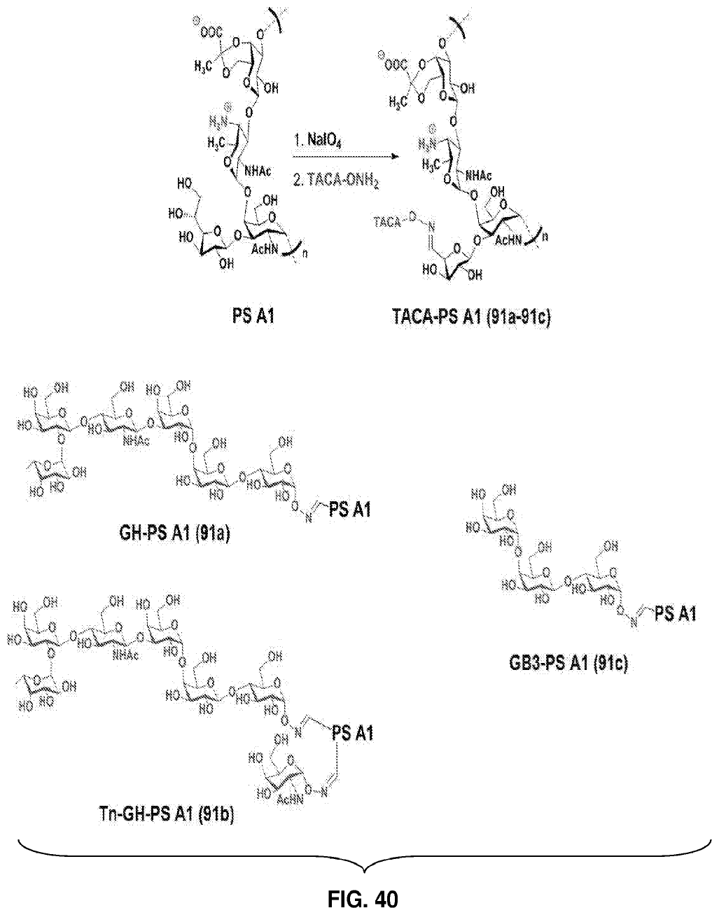

Antibody dependent cellular cytotoxicity (ADCC) is an in vivo and in vitro technique that can be used to determine the potency of antibody responses (FIG. 39A). Once an IgG antibody binds to target cells, the Fc portion of the antibody can recruit the Fc receptor on NK cells (either CD16 or FcRγIII) which triggers the release of granzymes to lyse the target cell. To further support the potency of the TF-PS B (FIG. 39B), ADCC was used to assess the activity of the anti-TF-PS B serum to initiate cell mediated killing. The anti-TF-PS B serum was able to produce 26% cytotoxicity, which was statistically significant compared to PS B, TF-BSA, and the control serum (both anti-TF and PBS). This result noticeably demonstrated the effectiveness of comparing cytotoxicity of TF-PS B to both PS B and TF-BSA. Another method to evaluate antibody responses is complement dependent cytotoxicity (CDC) (FIG. 39C). Similar to ADCC, once an antibody is bound to a target cell complement binds to the Fc portion of the antibody which initiates a membrane attack complex to lyse the cell. In FIG. 39D, the anti-TF-PS B serum did not produce any complement mediated toxicity. There are two explanations for the lack of complement mediated cytotoxicity: 1) the IgG antibodies out competed IgM antibodies for binding to MCF-7 and 2) some classes of IgG antibodies are not effective at fixing complement compared to IgM antibodies.

Conclusions

Zwitterionic polysaccharides can be a viable alternative to protein carriers in cancer vaccine development. Entirely carbohydrate based immune constructs for specific anti-carbohydrate immune responses, as opposed to heterogeneous protein constructs consisting of peptide(s)/protein(s) and sugars combined, are useful. One key feature of this approach is that the zwitterionic charges on polysaccharides 51 and 52, which are essential components for immune activation, are most likely due to the electrostatic similarities of peptides and specific uptake through C-type lectins. Therefore, using ZPSs as immunogens in cancer vaccine development can be supported through the innate and adaptive immune responses for ZPSs.

The immune response(s) generated from TF-PS B resulted in antibodies specific for the TF disaccharide, void of amino acids, chemical linkers or proteins. The majority of antibody isotypes obtained were IgM; their pentavalent nature allows for increased binding due to higher avidity towards glycans which can result in complement mediated killing. The generation of IgG1 and IgG2b isotypes indicates the activation of Th2 and Th1 mediated immunity, which is useful in antibody directed cellular cytotoxicity. This contrasts the immune response generated by TACA-PS A1, which induces a Th1/Th17 immunity. However, IgM/IgG antibodies generated by TF-PS B showed greater fluorescent binding events in flow cytometry than anti-PS B immunoglobulins (FIGS. 38A and 38C) by binding to TF expressing MCF-7 cells. Additionally, anti-TF-PS B antibodies showed a preference towards MCF-7 over HCT-116; it is known that MCF-7 cells have a higher expression level of TF (CD176) than do HCT-116 carcinomas. Collectively, the anti-TF PS B immune response was able to recognize the TF antigen in both flow cytometry and ELISA, which demonstrated ZPS-based tumor antigen conjugates can be a viable protein alternative for TACA based cancer vaccines.

To determine the efficacy of the ZPS-based tumor immune responses, data was compared with a TF-BSA protein conjugate (FIG. 36). The results indicated the protein construct was not as equally sufficient in generating higher immunological titers towards TF than the TF-PS B equivalent. These results demonstrate that using ZPSs as immunogens increases the immunogenicity of carbohydrate antigens by exploiting innate and adaptive immune responses. One advantage in using ZPS conjugates is bacterial polysaccharides can cross-link surface receptors on dendritic cells to promote efficient antigen uptake through large carbohydrate oligomers. PS B generates a distinct immune response differing from PS A1 as noted by the absence of expressed IgG3 antibodies that are correlated to a Th17 immune response. It is believed that this differentiation is based on varying interactions with CLRs, where PS A1 interacts with DC-SIGN and although currently not entirely understood, PS B may have interactions with other lectins that have a preference for N-acetylated sugars or even fucose. The importance of using TF-PS B as an immunogen, therefore, is to facilitate uptake on APCs and generate antibodies that can mitigate metastasis of TF containing carcinomas to promote tumor cell killing. The utility of anti-TF antibodies from TF-PS B may also assist in halting metastasis by preventing galectin-3 recognition and by promoting antibody directed cytotoxicity towards cancer cells. The comparison between TF-PS B and Tn-PS A1 is not a valid comparison due to the differences in carbohydrate antigens.

Experimental

Culturing B. fragilis and Purification of PS B (52)

20 L of B. fragilis was harvested after 48 h of growth and centrifuged at 4,000×g for 20 min at 4° C. in 500 mL centrifuge bottles. Cell supernatant was removed and the pellet was re-suspended in 500 mL of 0.15 M NaCl. Liquefied phenol (EMD Millipore) (500 mL) was added to the re-suspended cell pellet and stirred at 70° C. for 30 min. The aqueous layer was removed from the liquefied phenol using a separatory funnel. The aqueous layer was back extracted three times with diethyl ether and dialyzed with SnakeSkin™ dialysis tubing (10K MWCO). Crude bacterial lysate was treated with RNase (Sigma) and DNase (Sigma) in 0.1 M sodium acetate buffer (pH 4.5), followed by Pronase® (Roche) treatment (pH 7.0) and finally dialysis. The crude mixture was purified by size exclusion chromatography (Sephacryl S-300 HR) with elution buffer (0.5% sodium deoxycholate, 50 mM glycine, and 10 mM EDTA (pH 9.8)). Fractions were collected and analyzed using UV-spectroscopy; fractions were pooled if there was no absorbance at 260 and 280 nm. The elution buffer was removed by dialysis and crude samples were analyzed by ¹H NMR. The final step in purification required anion-exchange chromatography (DEAE-Sepharose) to separate the zwitterionic polysaccharides using Tris-HCl (pH 7.3) and a salt gradient from 0 M-2 M NaCl for elution of the polysaccharides was used. Purity of PS B was assessed by ¹H and ³¹P NMR.

Synthesis of Anomeric Aminooxy TF (53)

Synthesis of TF-ONH₂ was conducted using known methods.

Synthesis of TF-PS B (54)

Random oxidization of 1.0 mg of PS B using 10 mM of sodium periodate in 0.5 mL 0.1 M sodium acetate buffer pH 5.0 was accomplished by allowing the reaction to stir for 90 min in the dark, followed by quenching with 1 M KCl. TF-ONH₂ (53) 2.0 mg was then added to the solution of oxidized PS B and the reaction was allowed to stir overnight. TF-PS B was dialyzed and lyophilized. Conjugation was observed by oxime formation (7.4-8.0 ppm) using H¹ NMR (see below for spectral data).

TF-BSA (55)

Aminooxy TF (53) 5.0 mg was reacted with mercaptoaldehyde (56) for 18 h in sodium acetate buffer (pH 5.5) at room temperature and purified using Sephadex G-10 and deionized/distilled H₂O as the eluent. Fractions containing the TF-linker were lyophilized. 2.5 mg of (7) was deacetylated using Zemplen's method with NaOMe in methanol followed by base neutralization with DOWEX 50W×8-100 ion exchange resin. The solution was then filtered and concentrated under reduced pressure.

NMR and MS Analysis for Compound (57)

¹H NMR (600 MHz, D₂O): (E and Z isomers): δ 7.4 (dd, J₁=15.1 Hz, J₂=8.6 Hz, 1H_{E}), 5.3 (dd, J₁=13.9 Hz, J₂=4.0 Hz), 4.4-4.3 (m, 4H), 4.2 (dd, J₁=12 Hz, J₂=2.3 Hz, 1H), 4.0 (d, J₁=5.7 Hz, 1H), 3.9-3.8 (m, 2H), 3.8 (d, J=3.1 Hz, 2H), 3.6-3.5 (m, 9H), 3.5-3.5 (m, 4H), 3.4-3.4 (m, 2H), 3.1 (q, 3H), 3.0-2.9 (m, 3H), 2.3-2.2 (m, 4H), 1.9 (d, 7H), 1.5-1.4 (m, 5H), 1.2 (t, 6H), 0.8-0.8 (m, 6H). ¹³C NMR (150 MHz, D₂O): (E and Z isomers): δ 200.9, 174.6, 158.3, 104.7, 98.6, 76.9, 75.0, 72.5, 71.0, 68.6, 60.8, 52.1, 47.6, 46.6, 42.4, 42.0, 30.8, 30.0, 24.7, 21.9, 10.9. LRMS:ESI [M+(Na)⁺] calcd for 563.19 found 563.1.

Analysis of Compound (55)

2.0 mg of BSA-maleimide (Pierce Biotechnology) was dissolved in 0.3 mL of reaction buffer (1×PBS buffer with 0.1 M EDTA (pH 7.2). Compound 58 was then dissolved in 0.2 mL of reaction buffer and added to a solution containing BSA-maleimide. The reaction proceeded for 24 hr at 4° C. and was extensively dialyzed at 4° C. Conjugation was analyzed by MALDI-TOF (M/Z 90080.640). Mass loading was calculated using the following equation: (MW of TF-BSA−MW of BSA Mal)/(MW of TF-linker). It was determined that there were 34 molecules of TF-linker conjugated per BSA-maleimide.

Immunizations

Jax C57BL/6 male mice, 6 weeks, were obtained from Jackson Laboratories and maintained by the Department of Laboratory Animal Resources (DLAR). All animal protocols were performed in compliance with the relevant laws and institutional guidelines and have been approved by the Institutional Animal Care and Use Committee (IACUC) of the University of Toledo. Mice were immunized by intraperitoneal injections (i.p.) with 10 μg of TF-PS B, PS B and TF-BSA with and without TiterMax® Gold. Injections were performed on Day 0, 7, 14, and 28. Blood was collected and pooled in a BD Vacutainer® SST™ on Day 32 using a cardiac puncture technique to draw blood. Blood was allowed to clot and serum was separated in BD Vacutainer® SST™ using a manufacture protocol.

PS B poly-L-lysine (PS B-PLL) and TF-PS B poly-L-lysine (TF-PSB-PLL)

100 μg of PS B or TF-PS B was added to a test tube containing 0.5 mL of 0.01 M NaOH (0.001% phenolphthalein indicator) and 0.5 mg of cyanuric chloride. The mixture was vortexed for 1 min and 0.1 mL of 0.1% poly-L-lysine (PLL) was then added to the mixture, vortexed for 1 min and allowed to react for 3 h at 4° C. on a shaker. The conjugate was diluted to 30 mL with 0.1 M carbonate buffer (pH 9.2).

ELISA

Immulon™ 4 HBX 96 well plates (coated with either PS B/TF-PS B-PLL or TF-BSA) and maleic anhydride activated 96 well plates (coated with TF-ONH₂) (Thermo Scientific) were used to determine titers from immunized PS B, TF-PS B, and TF-BSA mice. The Immulon™ 4 HBX plates were coated with TF-BSA or TF-PSB/or PS B-PLL (3 μg/mL in 0.1 M carbonate buffer (pH 9.2). Maleic anhydride plates were coated with TF-ONH₂ (3) as per manufacture instructions. Plates were left at 37° C. for 1 h with shaking and then continued overnight at 4° C. The plates were then washed three times with washing buffer (1×TBS, 0.05% Tween 20, pH 7.3) and blocked with blocking buffer (2% BSA, 1×TBS, pH 7.3) and incubated for 1 h, followed by washing three more times with washing buffer. Anti-sera were initially diluted 1:300 for total antibody titers and 1:100 for IgG isotypes, then serially diluted in half-log₁₀ dilutions and incubated for 2 hr at 37° C. followed by washing three times with washing buffer Alkaline phosphatase secondary antibodies anti-(kappa, IgG) diluted (1:2000) and (IgM, IgG1, IgG2a, IgG2b, and IgG3) were purchased from (Southern Biotech) diluted (1:1000) and incubated for 1 h, followed by washing three times with washing buffer. PNPP tablets (Pierce) were dissolved in diethanolamine substrate buffer (pH 9.8) and then 100 μL was added to each well for 30 min for sufficient color to develop to detect secondary antibodies. The reaction was quenched with 2 M NaOH. Optical density measurements were obtained using a UV plate reader (Bio-Tek PowerWave HT) and the 96 well plates were read at 405 nm using Gen5 2.0 plate reading software. All assays were performed in triplicate. Titers were determined by regression analysis with half-log₁₀ dilutions plotted against absorbance. The titer cutoff value was set at 0.2 for titer determination. Statistically analysis from ELISAs for experimental groups were compared with the controls using paired t test using GraphPad Prism 6.

Flow Cytometry

MCF-7 and HCT-116 cells lines were provided by (Dr. Frederick Valeriote, Henry Ford Health Systems). Anti-sera were diluted to 1:200 with FACS buffer (1×PBS, 2% FBS, and 0.001% azide) and incubated with the cell lines (1×10⁶ cells) for 30 min on ice. Cells were washed with FACS buffer three times and incubated with secondary antibodies using either AlexaFluor® 488/647 and washed three times. Cells were analyzed by flow cytometry using BD FACSCalibur™ and data analysis obtained using FlowJo software.

Synthesis of STn-PS B (59)

Random oxidization of 1.0 mg of PS B using 10 mM of sodium periodate in 0.5 mL 0.1 M sodium acetate buffer pH 5.0 was accomplished by allowing the reaction to stir for 90 min in the dark, followed by quenching with 1 M KCl. 2.0 mg of STn-ONH₂ was then added to the solution of oxidized PS B and the reaction was allowed to stir overnight. TF-PS B was dialyzed and lyophilized. Conjugation was observed by oxime formation (7.4-8.0 ppm) using H¹ NMR (see below for spectral data).

Periodate-Rescorcinol Assay for Sialic Acid

A linear gradient of sialic acid, N-acetyl galactose, and galactose amine was generated from 40, 35, 30, 25, 20, 15, 10, 7.5, 5, 2.5, 1, and 0.5 μg. STn-PS B (59) and PS B (52) were added in triplicate in separate wells at 50 μg per well. 40 μL was placed in triplicate for each concentration in a 96 well plate. 10 μL of 5 mM NaIO₄ was placed in each well and incubated for 35 min at 4° C. making a final concentration of 1 mM. 100 μL of rescorinol solution (0.6 g of resorcinol in 100 mL of 17% HCl solution and 0.0025 mM of CuSO₄) was added to the well-plate and incubated for 60 min at 90° C. The unknowns were determined from the sialic acid concentration at 580 nM.

Percent Loading of STn-PS B

Sialic acid by weight was determined from the periodate rescorinol assay and STn percent loading was calculated by the following equation:

(Amount of sialic acid (μg) from assay)/(weight of glyconjugate)×(molecular weight STn/molecular weight of sialic acid)×100%.

Alexa Fluor® 488 Percent Loading

100 μg of oxidized PS B was reacted with 100 μg of Alex Fluor® 488-hydrazide (Molecular Probes) for 24 h in PBS buffer pH 7.4 followed by dialysis. The solution was lyophilized and re-dissolved with 100 μl of PBS. Optical density measurements were obtained using a UV plate reader (Bio-Tek PowerWave HT) and then the 96 well plates were read at 495 nm using Gen5 2.0 plate reading software. The percent loading of Alexa Fluor® 488 was determined using the manufacturer protocol(s) (Invitrogen Alex Fluor® 488 Protein labeling Kit). The following equation was used: Moles of dye per mol of PS B=A₄₉₄/(71000 cm⁻¹M⁻¹×PS B concentration).

### Example 6—Immunological Evaluation of Globo H-PS A1 Conjugates

Globo H is a unique ganglioside based hexasaccharide tumor associated carbohydrate antigen (TACA) and is anchored in tumor cells through a lipid ceramide. It is overexpressed in many tumor cells such as breast, ovarian, prostate, etc., and it was first identified on the MCF-7 cell line in 1984. Its hexasaccharide nature is unique and has recently been involved in clinical trials with Globo H conjugated to KLH or CRM 197, but to date no TACA based vaccine has been granted approval.

Globo H remains an important carbohydrate target not only because of the expression on breast cancers, but also its contribution to angiogenesis and expression on cancer stem cells (CSC), leading to tumor initiation and progression. Globo H has been shown to induce immunosuppression by shedding from the tumor and decreasing T and B cell populations by reducing Notch1 signaling. Therefore, targeting Globo H can be vital for the clearance of primary tumor cells and CSCs by halting tumor cell recurrence. Additionally, Globo H shares a common trisaccharide core (Galα1-4Galβ1-4Glc) structure with GB3, which is also expressed on CSCs but not on normal stem cells. The mechanism for increased expression of gangliosides is facilitated by glycosyltransferases A4GALT (GB3) and FUT1/FUT2 for Globo H. Therefore, not only would an effective vaccine be able to act as an angiogenesis inhibitor but also as a potent mediator of cytotoxicity by ADCC and CDC of CSC. Increasing the immunogenicity of TACAs is a common theme to clinically validate these targets, but the use of adjuvants remains essential to augment immune responses.

CLRs are an important part of carbohydrate based immunity (especially with ZPS) by promoting targeted carbohydrate based immunogens. However, focusing on certain carbohydrate antigens can modulate the immune responses by promoting proinflammatory cytokines such as IL-6 and increased antigen uptake. These effects were noted when a unimolecular bivalent Tn-TF-PS A1 construct was able to increase the immune response towards the TF antigen, when compared to the monovalent TF-PS A1 alone. Conjugating Globo H to a ZSP can effectively produce a vaccine that has targeted function towards dendritic cells due to the multivalent binding effects.

Results and Discussion

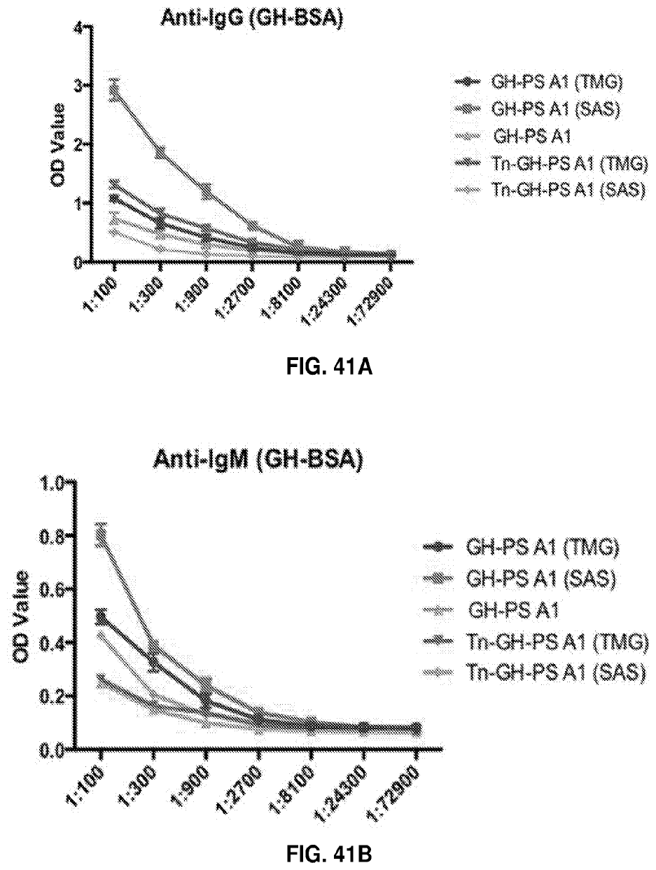

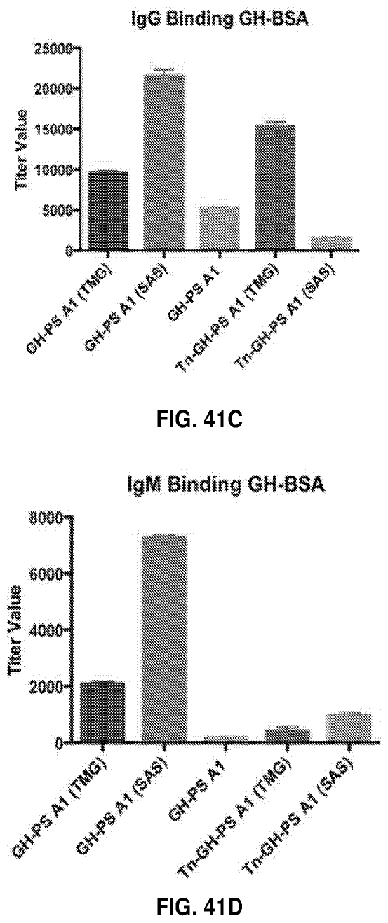

PS A1 was oxidized using sodium periodate and three separate conjugates were semi-synthetically prepared through an oxime link. The formation of the oxime linkage provides greater hydrolytic stability than hydrazones, hydrazides, and imines due to the electronegativity of the oxygen compared to either nitrogen or carbon. This added stability is important in ensuring the TACA-ONH₂ is tethered to PS A1 after being subjected to acidic lysosomes en route for presentation to T cells by MHC II. Globo H-PS A1 (GH-PS A1) and a unimolecular bivalent construct Tn-GH-PS A1 was injected into C57/BL6 mice and the immunological evaluation was assessed with and without Sigma Aldrich Adjuvant (SAS) or TiterMax Gold (TMG). SAS is a mixture including monophosphloryl lipid A (MPLA), a TLR 4 agonist, and synthetic trehalose dicorynomycolate (STDCM), which binds to C-type lectins, minicle, and dectin-2, which increases production of proinflammatory cytokines. TMG is a potent oil in water emulsion which provides slow release of antigens and its main component CRL-8300, is composed of conjugated copolymer of polyethylene oxide and polypropylene oxide. FIGS. 41A-41D display the selective anti-Globo-H immune response generated from a series of Globo-H based PS A1 incorporated into constructs with different adjuvants. Examination of the GH-PS A1 constructs revealed exceptional IgG and IgM specificity towards Globo-H-BSA. The GH-PS A1 (SAS) had exceptional anti-IgG and anti-IgM binding with a titer value of 22,000 and 7,300, respectively. Additionally, GH-PS A1 (TMG) showed potent anti-IgG titers with a titer value of 9,700. The difference of the administration of adjuvant between SAS compared to TMG had a significant three-fold effect on the amount of antibody towards GH-BSA.

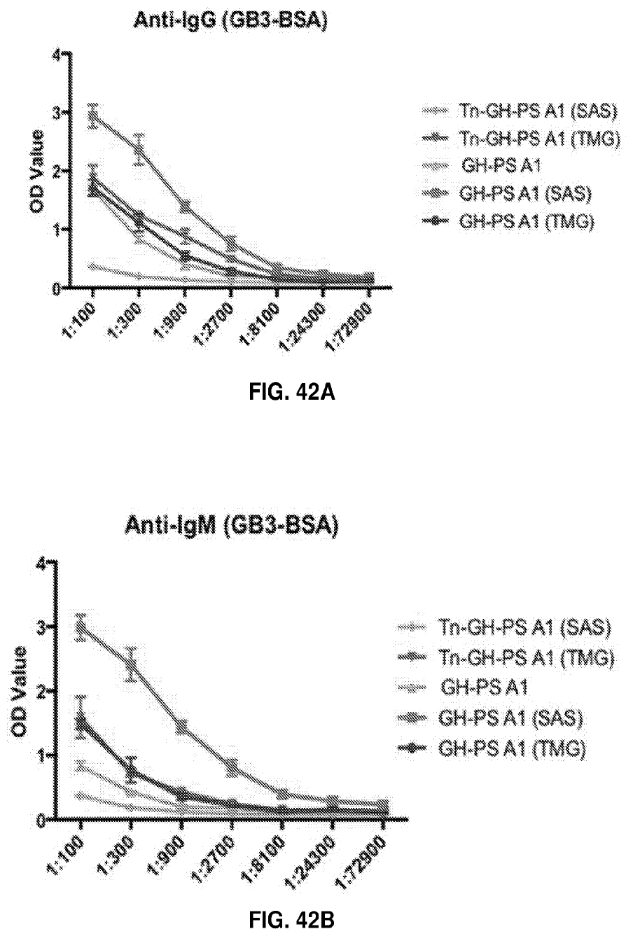

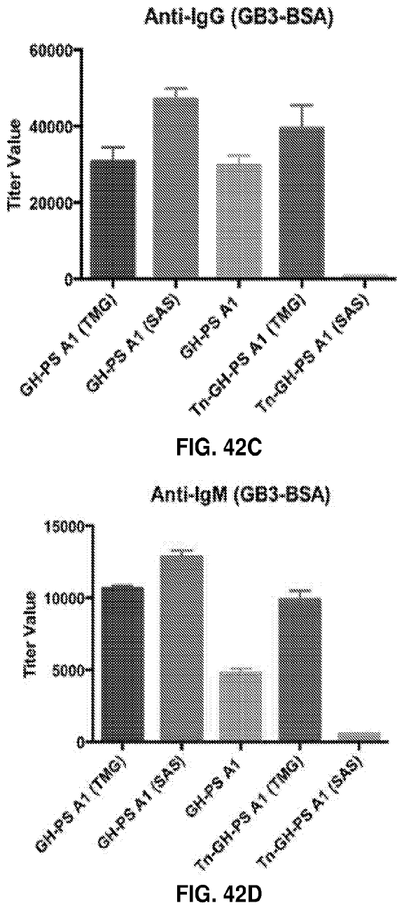

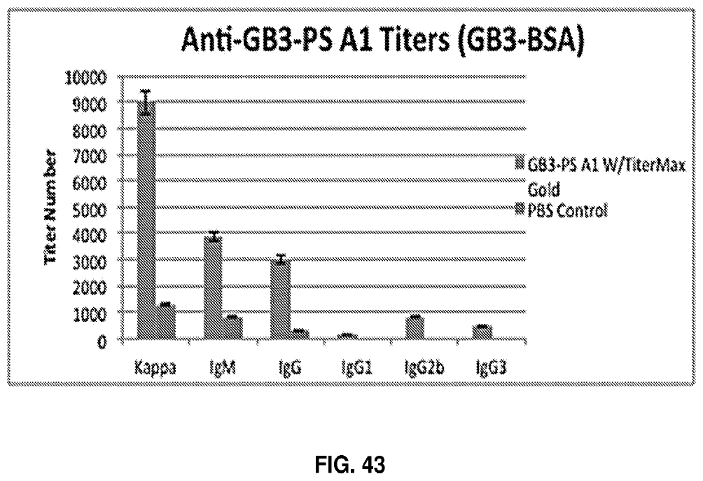

When comparing both adjuvants while investigating the unimolecular bivalent construct Tn-GH-PS A1, an interesting phenomenon occurred. The Tn-GH-PS A1 with TMG had an increased anti-IgG titer of 15,700 compared to 9,700 from GH-PS A1 with TMG. This result indicates the presence of Tn alone can augment the selectivity and specificity of the anti-IgG immune response towards GH-BSA. However, when Tn-GH-PS A1 was administered with SAS, there was an enormous reduction of both anti-IgG and anti-IgM. Without wishing to be bound by theory, it is believed that simultaneous activation of CLRs (DC-SIGN and DCIR) reduces activation and presentation to T cells by APCs. Another interesting result that occurred through the immunological evaluation of GH-PS A1 constructs, was the higher generated immune response against GB3-BSA (FIGS. 42A-42D). The immune response was notably higher for all of the anti-serum generated against GH-PS A1 to GB3-BSA. Since GB3 is a part of the core structure of Globo H, it is plausible Globo H will become fragmented due to radical nitric oxide degradation. This ultimately leads to a fragmented portion of Globo H presented to T cells to assist in antibody generation. For comparison, this resulted in close to a two-antibody response generated against GB3-PS A1 (TMG) to compare to GH-PS A1. While there is a substantial titer for total antibody response (kappa), the antibody response generated as pIgG and pIgM antibodies is substantially decreased in comparison to anti-GH-PS A1 constructs (FIGS. 42A-42D). These results validate the immune modulating properties of a terminal fucose (Globo H) compared to terminal galactose (GB3). A comparison towards Tn-PS A1 (terminal GalNAc) and TF-PS A1 (terminal Gal) shows the addition of galactose containing moieties seen with the TF antigen dampening the immune response by increased IL-10 values.

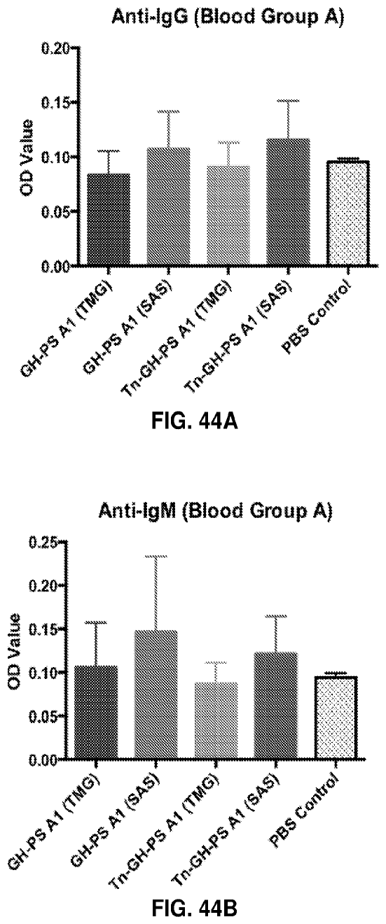

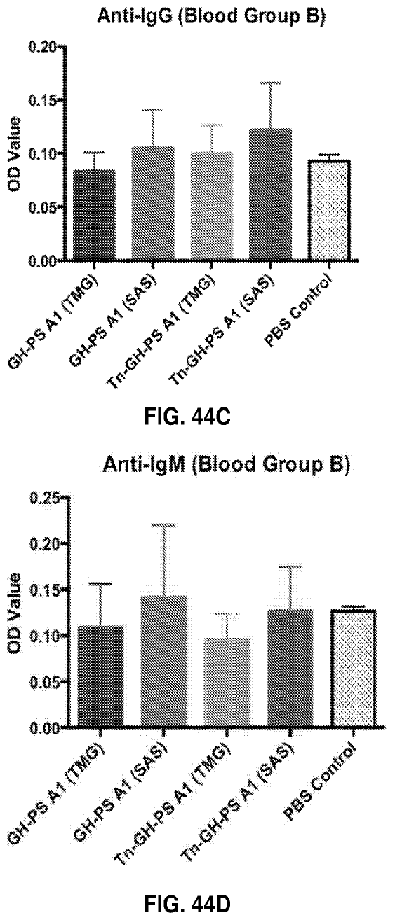

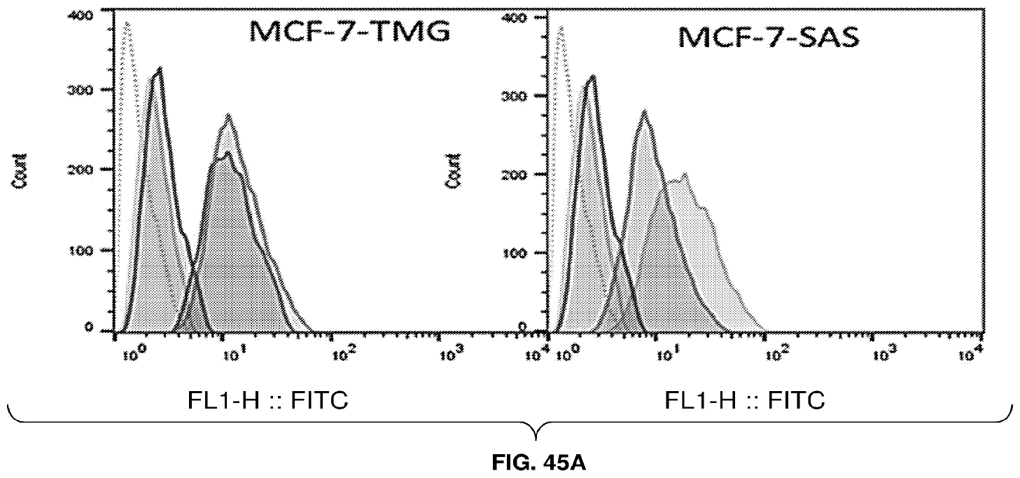

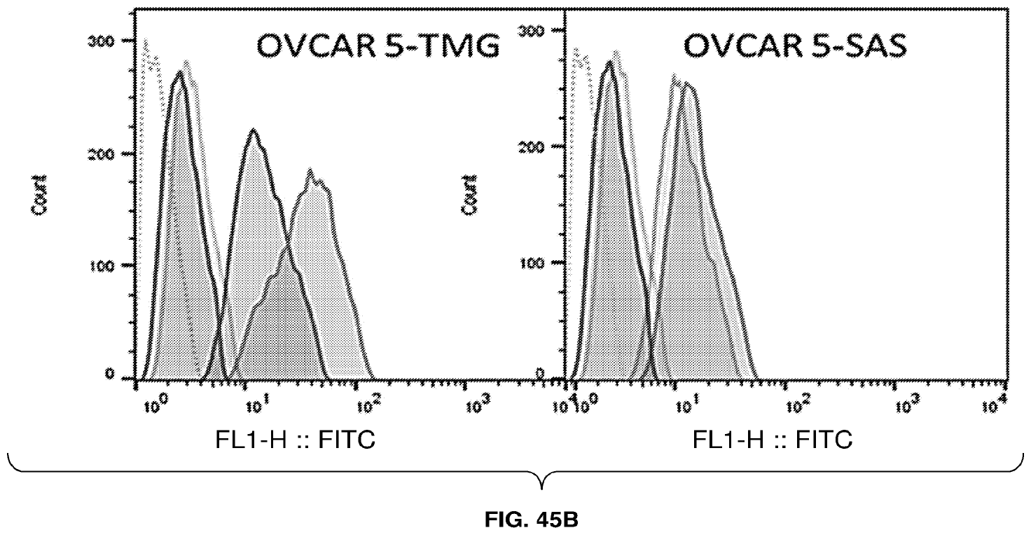

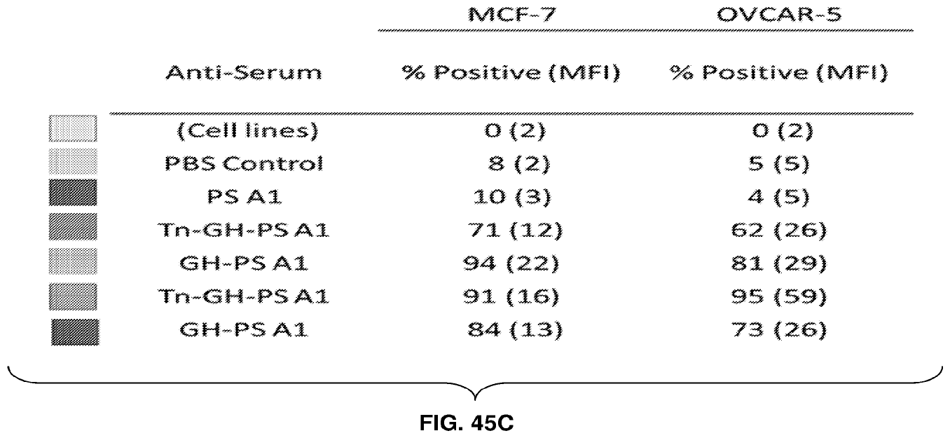

A particular concern of creating an immune response with a construct containing both fucose and N-acetyl galactosamine is there could be immense cross reactivity with blood group A and blood group B. Therefore, anti-serum from the GH-PS A1 based constructs were screened for binding to both of the blood groups in ELISA (FIGS. 44A-44D). Both the GH-PS A1 (TMG and SAS) had relatively low anti-IgG and anti-IgM binding to blood group A (BGA) and blood group B (BGB) with optical density value less than 0.2. Additionally, the Tn-GH-PS A1 (TMG and SAS) were analogous to GH-SAS where there was minimal anti-IgG and anti-IgM cross reactivity towards BGA and BGB. This result indicates that there is not a concern with large immune responses towards Globo H and the potential of cytotoxicity of red blood cells. Flow cytometry was then used to determine the IgG response binding to human tumor cell lines MCF-7 (breast) and OVCAR-5 (ovarian) (FIGS. 45A-45C). The anti-serum from the GH-PS A1 constructs and respective adjuvant formulations were individually used to determine binding to cancer cells. Analogous to FIGS. 41A-41D and FIGS. 42A-42D, the anti-serum generated showed good binding to MCF-7 and OVCAR-5. When specifically examining the binding of the TMG series (GH-PS A1 and Tn-GH-PS A1), both anti sera showed exceptional binding to MCF-7 with 84% positive shift in fluorescent intensity (GH-PS A1 TMG) and 91% positive shift in fluorescent intensity (Tn-GH-PS A1 TMG) compared to the controls of PBS (8%), PS A1 (10%), and auto-fluorescence of the cell line alone. Tn-GH-PS A1 TMG had the greatest fluorescent intensity when binding to OVCAR-5, which makes an interesting discovery compared to Tn-GH-PS A1 SAS. The difference in binding may be contributed to over stimulation of CLRs with the adjuvant of SAS leading to less effective antibody binding responses.

Similar results were observed with the TMG series binding with OVCAR-5 with a 95% positive (Tn-GH-PS A1 TMG) and 84% positive with (GH-PS A1 TMG) compared to the controls of PBS (5%) and PS A1 (4%). When examining the SAS series, as expected, the Tn-GH-PS A1 SAS showed the lowest anti-IgG binding with 71% binding to MCF-7 and 62% OVCAR-5. However, GH-PS A1 SAS showed the highest binding to MCF-7 cell line with 94% positive fluorescent anti-IgG binding events and 81% binding to OVCAR-5.

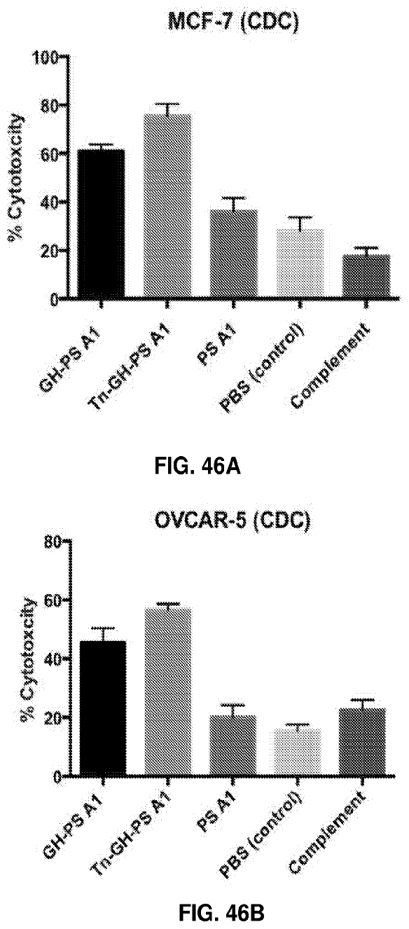

For determining the LDH assay (FIGS. 46A-46B), the anti-sera that demonstrated the highest binding in ELISA and flow cytometry were selected for their potential to mediate complement dependent cytotoxicity. The two that were investigated were GH-PS A1 SAS and Tn-GH-PS A1 TMG. Tn-GH-PS A1 TMG demonstrated superior cytotoxicity towards both MCF-7 and OVCAR-5 tumor cells with 79% and 58%, respectively. Additionally, these results are significant compared to the cytotoxicity from PS A1 serum towards MCF-7 (40%) and OVCAR-5 (17%).

The combined effects from having multiple TACAs on a unimolecular bivalent construct lead to greater binding to tumor cells. The Globo H-PS A1 SAS also had significant binding to MCF-7 and OVCAR-5 with 63% and 49%, respectively. Collectively, both the Tn-GH-PS A1 TMG and GH-PS A1 demonstrated excellent cytotoxicity between cell lines.

Conclusions

The synthesis of Globo H-PS A1 and Tn-GH-PS A1 and subsequent immunizations have generated high immune responses towards Globo H which resulted in tumor cell binding and high cytotoxicity of both MCF-7 and OVCAR-5. The advantages of using the ZPS platform are related to the entirely carbohydrate vaccine construct with entirely carbohydrate specificity and targeted uptake by dendritic cells through CLRs.

The results indicated the use of adjuvants play a major effect in the immunogenicity in both Globo H-PS A1 and Tn-Globo H-PS A1. The use of SAS had a significant impact on the anti-IgG response from GH-PS A1 with a titer of 22,000 compared to 9,700 from GH-PS A1. This indicates the proinflammatory cytokines generated from MPLA and STDCM assist in producing a larger immunological titer. However, the same proinflammatory environment from SAS did not produce the same desired results with Tn-GH-PS A1. In fact, using SAS with Tn-GH-PS A1 dampened the IgG antibody response nearly tenfold in comparison to the response generated from Tn-GH-PS A1 TMG. The difference in binding may be contributed to the over stimulation of CLRs with the adjuvant of SAS leading to less effective antibody binding responses. An interesting phenomena has been demonstrated when a ligand interacted simultaneously with both DC-SIGN and dendritic cell immunoreceptor (DCIR), showing reduced activation and presentation to T cells. It can be concluded that multiple interactions from C-type lectins and multiple interactions of TLRs can ultimately affect T cell presentation and subsequent immune response.

Analysis through flow cytometry of the GH-based PS A1 constructs revealed high recognition of both tumor cell lines MCF-7 and OVCAR-5 due to the cell lines expression of both Tn and Globo H.

Experimental

GH-PS A1 (91a)

1.0 mg of PS A1 was oxidized using 1 mM sodium periodate in sodium acetate buffer pH 5.0 for 90 min in the dark. Excess sodium periodate was quenched with KCl and desalted using centrifugal filter (10 kDa MWCO). 1.3 mg of Globo H-ONH₂ reacted with oxidized PS A1 for 16 h. The resulting reaction was desalted using centrifugal filter (10 kDa MWCO). ¹H NMR was used to determine oxime formation.

Bivalent Tn-GH-PS A1 (91b)

1.0 mg of PS A1 was oxidized using conditions as described above. A1:1 molar ratio of 1.1 mg of Globo H-ONH₂ and 0.25 mg of Tn-ONH₂ was reacted with freshly oxidized PS A1. Excess salts and by products were removed by centrifugal filtration. ¹H NMR was used to determine two separate oxime formation.

GB3-PS A1 (91c)

1.0 mg of PS A1 was oxidized using conditions as described above. 1.2 mg of GB3-ONH₂ was reacted with 1.0 mg of oxidized PS A1 for 16 h. The reaction was dialyzed and lyophilized. ¹H NMR was used to determine oxime formation.

Immunizations

Individual GH, Tn-GH, or GB3-PS A1 constructs (20 μg) were mixed in a 1:1 ratio of 50 uL of TiterMax® Gold and injected into 7 wk old C57BL/6 mice (Jackson Laboratory) (each construct was administered individually—not mixed). Mice groups (n=5) were immunized by intraperitoneal injections (i.p.) on day 0, 14, 28, 42. Blood sera were obtained using a cardiac puncture technique on day 52.

Vaccinations with Sigma Adjuvant System

Individual GH and Tn-GH-PS A1 constructs (20 μg) were mixed in a 1:1 ratio of 100 μL of Sigma Adjuvant System (Sigma-Aldrich) and injected into 7 wk old C57BL/6 mice (Jackson Laboratory) (each construct was administered individually not mixed). Mice groups (n=5) were immunized by intraperitoneal injections (i.p.) on day 0, 21, 42, per manufactures instructions. Blood sera were obtained using a cardiac puncture technique on day 52.

Enzyme Linked Immunosorbent Assay (ELISA)

Either GH-BSA, GB3-BSA, blood group A/or blood group B was coated on Immulon® Microtiter™ 4 HBX 96 well plates using 3 μg/mL in carbonate buffer (pH 9.2) and then the plates were incubated for 18 h at 4° C. ELISA procedures described above were followed.

Synthesis of GH-Thio Linker

3.0 mg of Globo H-ONH₂ was reacted with 1.0 mg of 3-oxopropyl ethanethioate (mercaptoaldehyde) for 24 h. The reaction was purified by Sephadex G-10 and deionized/distilled H₂O as the eluent. Fractions containing the GH linker were lyophilized. ¹H NMR (D₂O, 600 MHz): δ=7.56 (t, J=6.2 Hz, 1H), 5.34 (d, J=3.7 Hz, 1H), 5.12 (d, J=4.4 Hz, 1H), 4.75-4.83 (m, 8H), 4.74 (br. s., 7H), 4.64-4.70 (m, 11H), 4.49-4.53 (m, 1H), 4.40-4.46 (m, 3H), 4.27-4.31 (m, 1H), 4.10-4.15 (m, 3H), 3.98-4.01 (m, 1H), 3.90-3.93 (m, 2H), 3.77-3.89 (m, 9H), 3.71-3.76 (m, 7H), 3.51-3.70 (m, 26H), 3.49-3.50 (m, 1H), 3.48 (t, J=2.0 Hz, 1H), 2.95-3.03 (m, 2H), 2.40-2.50 (m, 1H), 2.24-2.29 (m, 3H), 1.91-1.95 (m, 4H), 1.07-1.13 ppm (m, 4H). ¹³C NMR (D₂O, 151 MHz): δ=201.1, 201.0, 174.3, 104.0, 103.2, 102.0, 100.4, 99.2, 91.5, 78.2, 77.1, 76.3, 76.1, 75.4, 75.0, 74.6, 73.5, 72.1, 71.8, 71.7, 71.1, 70.8, 70.2, 70.1, 69.4, 69.2, 69.0, 68.4, 68.0, 67.8, 66.7, 60.9, 60.3, 59.4, 51.6, 30.0, 29.9, 29.2, 25.8, 25.4, 25.2, 22.2, 18.5, 15.3 ppm.

Globo H-BSA

2.0 mg of Globo H-thiol linker was deacetylated by a solution of concentrated K₂CO₃ for 1.5 h. Zemplen conditions were not used because Globo H is insoluble in MeOH. Globo H-thiol linker and reacted with freshly prepared BSA-Malemide (procedure described previously) in PBS buffer with 1 mM EDTA pH 7.2. After 16 h at 4 C, the reaction was dialyzed 10,000 MWCO. Conjugation was confirmed with MALDI-TOF (92249.938) for a total conjugation of 15.5%.

GB3 Thiol Linker

2.0 mg of GB3-ONH₂ reacted with 1.5 mg of mercaptoaldehyde for 18 h in sodium acetate buffer (pH 5.5) at room temperature and purified using Sephadex G-10 and deionized/distilled H₂O as the eluent. Fractions containing the −GB3 linker were lyophilized. 2.5 mg of (97) was deacetylated using Zemplen's method with NaOMe in methanol followed by base neutralization with DOWEX 50W×8-100 ion exchange resin. The solution was then filtered and concentrated under reduced pressure. ¹H NMR (D₂O, 600 MHz): δ=7.52 (dd, J=8.5, 3.7 Hz, 1H), 5.46 (t, J=4.2 Hz, 1H), 4.96 (d, J=3.4 Hz, 1H), 4.54 (dd, J=7.7, 6.5 Hz, 1H), 4.37 (t, J=6.2 Hz, 1H), 4.03-4.07 (m, 3H), 3.90-3.97 (m, 4H), 3.82-3.90 (m, 6H), 3.80 (br. s., 2H), 3.69-3.78 (m, 9H), 3.57-3.62 (m, 1H), 2.97-3.15 (m, 1H), 2.42 (td, J=9.2, 4.6 Hz, 1H), 2.38 (d, J=3.2 Hz, 3H), 1.51-1.68 (m, 1H), 0.90 ppm (dt, J=15.0, 7.5 Hz, 4H). ¹³C NMR (D₂O, 151 MHz): δ=200.9, 200.8, 181.5, 158.6, 103.1, 100.3, 99.2, 92.4, 78.1, 78.0, 77.3, 77.3, 75.4, 72.1, 71.6, 70.9, 70.8, 70.8, 70.3, 70.2, 69.1, 68.9, 68.5, 60.4, 60.3, 59.4, 42.2, 42.1, 30.8, 29.9, 29.9, 24.8, 24.6, 23.2, 10.8, 10.7 ppm.

Flow Cytometry

MCF-7 and OVCAR-5 was cultured in 10% FBS RPMI 1640. 1.0×10⁶ cells of each cell line was incubated at 4 C for 1 h in the dark with 1:50 dilution of the following separate anti-serums (PBS control, PS A1, Globo H-PS A1, Tn-Globo H-PS A1). The cells were washed three times in FACs buffer (2% FBS in PBS, 0.001% sodium azide) by centrifuging at 1000 rpm. 100 μL Anti-IgG Alexa Fluor 488 (1:50 dilution) was added to the cells and incubated at 4 C in the dark for 1 h followed by three washes with FACS staining buffer. The cells were fixed with freshly prepared 1% paraformaldehyde and obtained using BD Biosciences FACsCaliber by the University of Toledo Core flow cytometry facility. FlowJo FACs analysis was used to analyze the data.

Certain embodiments of the compositions and methods disclosed herein are defined in the above examples. It should be understood that these examples, while indicating particular embodiments of the invention, are given by way of illustration only. From the above discussion and these examples, one skilled in the art can ascertain the essential characteristics of this disclosure, and without departing from the spirit and scope thereof, can make various changes and modifications to adapt the compositions and methods described herein to various usages and conditions. Various changes may be made and equivalents may be substituted for elements thereof without departing from the essential scope of the disclosure. In addition, many modifications may be made to adapt a particular situation or material to the teachings of the disclosure without departing from the essential scope thereof.

What is claimed is:

1. A monoclonal antibody comprising:
a light chain amino acid sequence of:
[SEQ ID NO: 1]
CAAATTGTTCTCACCCAGTCTCCAGCAATCATGTCTGCATCTCCAGGGGA
GAAGGTCACCATGACCTGCAGTGCCAGCTCAAGTGTAAGTTACATGCACT
GGTACCAGCAGAAGTCAGGCACCTCCCCCAAAAGATGGATTTATGACACA
TCCAAACTGGCTTCTGGAGTCCCTGCTCGCTTCAGTGGCAGTGGGTCTGG
GACCTCTTACTCTCTCACAATCAGCAGCATGGAGGCTGAAGATGCTGCCA
CTTATTACTGCCAGCAGTGGAGTAGTAACCCGCTCACGTTCGGTGCTGGG
ACCAAGCTGGAGCTGAAA;
and
a heavy chain amino acid sequence of:
[SEQ ID NO: 2]
CAGATCCAGTTGGTACAGTCTGGACCTGAGCTGAAGAAGCCTGGAGAGAC
AGTCAAGATCTCCTGCAAGGCTTCTGGGTATACCTTCACAACCTATGGAA
TGAGCTGGGTGAAACAGGCTCCAGGAAAGGGTTTAAAGTGGATGGGCTGG
ATAAACACCTACTCTGGAGTGCCAACATATGCTGATGACTTCAAGGGACG
GTTTGCCTTCTCTTTGGAAACCTCTGCCAGCACTGCCTATTTGCAGATCA
ACAACCTCAAAAATGAGGACACGGCTACATATTTCTGTGCAAGACATTAC
TACGGAGGGGCTTACTGGGGCCAAGGGACTCTGGTCACTGTCTCTGCA.

2. A composition comprising a murine monoclonal antibody which (i) binds to a glycoside portion of a Tn antigen, and (ii) has the IgM isotype.

3. The composition of claim 2, wherein the composition is substantially free of additional peptides or proteins.

4. A composition comprising an antibody raised against an entirely carbohydrate immunogen.

5. The composition of claim 4, wherein the antibody is a mononclonal IgM antibody.

6. A test device, kit, or strip comprising the monoclonal antibody of claim 1.

7. The test device, kit, or strip of claim 6, wherein the monoclonal antibody is labeled with one of an enzyme, a fluorescent material, a chemiluminescent material, biotin, avidin, or a radioactive isoptope.

8. A pharmaceutical composition comprising:
a monoclonal antibody of claim 1; and
a pharmaceutically acceptable carrier, diluent, or adjuvant.

9. A composition comprising a STn-PS A1 construct having Formula I: 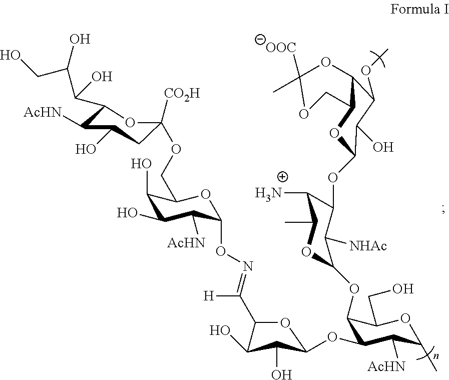
and salts, stereoisomers, racemates, hydrates, solvates, and polymorphs thereof.

10. A composition comprising a carbohydrate immunogen having Formula II: 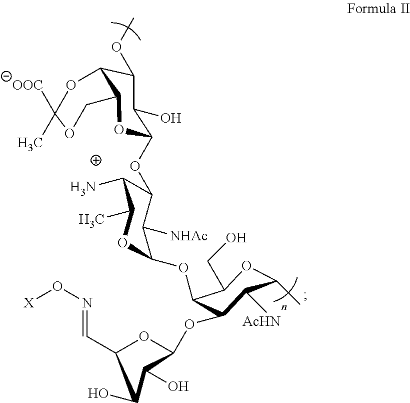
wherein X is selected from the group consisting of TF, Tn-TF, Gb3, and Globo H;
and salts, stereoisomers, racemates, hydrates, solvates, and polymorphs thereof.

11. A vaccine composition comprising:
an entirely carbohydrate immunogen comprising a zwitterionic polysaccharide conjugated to an STn antigen, a TF antigen, a Globo H antigen, or a conjugate of TF and Tn antigens; and
a pharmaceutically acceptable carrier, diluent, or adjuvant.

12. A method of treating, preventing, or ameliorating a cancer, the method comprising administering an effective amount of a pharmaceutical composition of claim 8 to a subject in need thereof, and treating, preventing, or ameliorating a cancer in the subject.

13. The method of claim 12, wherein the cancer is breast cancer.

14. A method of treating, preventing, or ameliorating a cancer, the method comprising administering an effective amount of a vaccine composition of claim 11 to a subject in need thereof, and treating, preventing, or ameliorating a cancer in the subject.

15. The method of claim 14, wherein the cancer is breast cancer.

16. A method of treating, preventing, or ameliorating a cancer, the method comprising administering monoclonal antibodies to a subject in need thereof, and treating, preventing, or ameliorating a cancer in the subject, wherein the monoclonal antibodies are generated from an immune response to an entiretly carbohydrate immunogen, and the monoclonal antibodies are specific and selective for glycosides of a tumor-associated carbohydrate antigen (TACA).

17. The method of claim 16, wherein the monoclonal antibodies are IgM antibodies.

18. The method of claim 16, wherein the cancer is breast cancer.

19. A method of generating monoclonal antibodies comprising:
administering an immunogen comprising an entirely carbohydrate construct to an animal to provoke an immune response in the animal and generate antibodies against the entirely carbohydrate construct, wherein the entirely carbohydrate construct comprises a zwitterionic polysaccharide conjugated to a tumor-associated carbohydrate antigen (TACA);
harvesting B cells from the animal;
fusing the harvested B cells with B cell cancer cells to produce hybridoma cells;
culturing the hybridoma cells; and
harvesting monoclonal antibodies from the cultured hybridoma cells, wherein the monoclonal antibodies are selective for glycosides of the TACA.

20. The method of claim 19, wherein the monoclonal antibodies are selective for a Tn antigen.

21. The method of claim 19, wherein the animal is a mouse.

22. The method of claim 19, wherein the entirely carbohydrate construct is a Tn-PS A1 construct.

23. The method of claim 19, further comprising humanizing the monoclonal antibodies.

24. The method of claim 19, further comprising producing an antibody chimera from the monoclonal antibodies.

25. The method of claim 19, further comprising converting the monoclonal antibodies into IgG antibodies.
